(19) United States
(12) Patent Application Publication
(10) Pub. No.: US 2024/0218468 A1
(43) Pub. Date: Jul. 4, 2024

(54) METHODS OF DIAGNOSIS OF RESPIRATORY VIRAL INFECTIONS

(71) Applicant: Inflammatix, Inc., Sunnyvale, CA (US)

(72) Inventors: Timothy Elisha Sweeney, Sunnyvale, CA (US); Yudong He, Sunnyvale, CA (US); Rushika Pandya, Sunnyvale, CA (US)

(21) Appl. No.: 18/557,784

(22) PCT Filed: May 11, 2022
(86) PCT No.: PCT/US22/28703

§ 371 Date: Oct. 27, 2023

Related U.S. Application Data
(60) Provisional application No. 63/187,337, filed on May 11, 2021.

(51) Int. Cl. C12Q 1/70

(52) U.S. Cl. CPC C12Q 1/701; C12Q 2600/106; C12Q 2600/158

(57) ABSTRACT

Systems, methods, compositions, apparatuses, and kits for determining the viral infection status of subjects using respiratory samples, and for determining effective triage strategies for such subjects, are provided herein. The disclosed methods and compositions involve biomarkers identified from the application of a machine learning workflow to viral training data from respiratory samples. The biomarkers allow the calculation of a score that can be used to determine the viral infection status of the subjects.

### CROSS-REFERENCES TO RELATED APPLICATIONS

This application claims priority to U.S. Provisional Application No. 63/187,337, filed May 11, 2021, the disclosure of which is hereby incorporated by reference in its entirety for all purposes.

### BACKGROUND

Acute respiratory viral infections are not only a common cause of illness, but also contribute to a substantial amount of mortality in children and adults. Any new diagnostic test needs to be more accurate as well as easy to use. Nasal swabs are commonly gathered to test directly for viral or bacterial pathogens, but this method suffers from colonizer false-positives, and is limited to only those pathogens present in the test.

The host immune response represented in the whole blood transcriptome has been repeatedly shown to diagnose presence, type, and severity of infections. By leveraging clinical, biological, and technical heterogeneity across multiple independent datasets, we have previously identified a conserved host response to respiratory viral infections that is distinct from bacterial infections and can identify asymptomatic infection. It is burdensome and not economical, however, to test blood samples from patients presenting for respiratory viral infections.

There is a need for new, safe, convenient, and accurate methods for diagnosing respiratory viral infections in patients. The present disclosure satisfies this need and provides other advantages as well.

### BRIEF SUMMARY

In one aspect, the present disclosure provides a method of administering medical care to a subject presenting one or more symptoms of a respiratory viral infection, the method comprising: (i) obtaining a respiratory sample from the subject; (ii) measuring expression levels of one or more biomarkers in the sample, wherein the one or more biomarkers comprise at least one biomarker from Table 2 or Table 3, or one pair of biomarkers from Table 4; and (iii) generating a viral score based on the measured expression levels of the biomarkers in the sample, wherein a viral score that exceeds a threshold value indicates that the subject has a viral infection.

In some embodiments of the method, the one or more biomarkers comprise at least one biomarker from Table 3. In some embodiments the one or more biomarkers comprise at least one pair of biomarkers from Table 4. In some embodiments, the method further comprises: (iv) determining the subject has a viral infection based on the viral score exceeding the threshold value; and (v) administering medical care to the subject to treat the viral infection based on the viral score. In some embodiments, the method further comprises: (iv) determining the subject does not have a viral infection based on the viral score not exceeding the threshold.

In some embodiments of the method, the respiratory sample is selected from the group consisting of nasal, nasopharyngeal, oropharyngeal, oral, or saliva sample. In some embodiments, the method further comprises detecting the presence or absence of one or more viruses in the sample. In some embodiments, the presence or absence of the one or more viruses is detected using a nucleic acid amplification test (NAAT). In some embodiments, the expression of the biomarkers is detected using qRT-PCR or isothermal amplification. In some embodiments, the isothermal amplification method is qRT-LAMP. In some embodiments, the expression of the biomarkers is detected using a NanoString nCounter. In some embodiments, the method comprises measuring the expression of 1, 2, 3, 4, 5, 6, 7, 8, 9, or 10 biomarkers in the sample. In some embodiments, the one or more biomarkers comprise IFITM1, TLNRD1, CDKN1C, INPP5E, and TSTD1.

In some embodiments of the method, the medical care comprises administering organ-supportive therapy, administering a therapeutic drug, admitting the subject to an ICU or other hospital ward, or administering a blood product. In some embodiments, the organ-supportive therapy comprises connecting the subject to any one or more of a mechanical ventilator, a pacemaker, a defibrillator, a dialysis or a renal replacement therapy machine, or an invasive monitor selected from the group consisting of a pulmonary artery catheter, arterial blood pressure catheter, and central venous pressure catheter. In some embodiments, the therapeutic drug comprises an immune modulator, an antiviral agent, a coagulation modulator, a vasopressor, or a sedative. In some embodiments, the respiratory viral infection is selected from the group consisting of adenovirus, coronavirus, human metapneumovirus, human rhinovirus (HRV), influenza, parainfluenza, picornavirus, and respiratory syncytial virus (RSV). In some embodiments, the viral infection is a SARS-COV-2 infection. In some embodiments, the coronavirus is coronavirus OC43, coronavirus NL63, coronavirus 229E, or coronavirus HKU1.

In another aspect, the present disclosure provides test kit for detecting the expression levels of one or more biomarkers in a respiratory sample from a subject with one or more symptoms of a respiratory viral infection, wherein the biomarkers comprise at least one biomarker from Table 2 or Table 3, or one pair of biomarkers from Table 4.

In some embodiments, the test kit comprises a microarray. In some embodiments, the kit comprises an oligonucleotide for each of the one or more biomarkers, wherein each of the oligonucleotides hybridizes to one of the biomarkers. In some embodiments, the biomarkers comprise IFITM1, TLNRD1, CDKN1C, INPP5E, and TSTD1. In some embodiments, the kit comprises an oligonucleotide that hybridizes to IFITM1, an oligonucleotide that hybridizes to TLNRD1, an oligonucleotide that hybridizes to CDKN1C, an oligonucleotide that hybridizes to INPP5E, and an oligonucleotide that hybridizes to TSTD1. In some embodiments, the kit is for detecting 1, 2, 3, 4, 5, 6, 7, 8, 9, 10, or more biomarkers. In some embodiments, the kit further comprises one or more reagents for performing q-RT-PCR, qRT-LAMP, or NanoString nCounter analysis. In some embodiments, the respiratory viral infection is selected from the group consisting of adenovirus, coronavirus, human metapneumovirus, human rhinovirus (HRV), influenza, parainfluenza, picornavirus, and respiratory syncytial virus (RSV). In some embodiments, the viral infection is SARS-COV-2. In some embodiments, the coronavirus is coronavirus OC43, coronavirus NL63, coronavirus 229E, or coronavirus HKU1. In some embodiments, the kit further comprises instructions to calculate a viral score based on the levels of expression of the biomarkers in the respiratory sample from the subject, the score correlating with the likelihood that the subject has a respiratory viral infection.

In another aspect, the present disclosure provides a computer product comprising a non-transitory computer readable medium storing a plurality of instructions that when executed cause a computer system to perform the method of any one of the herein-described methods.

In another aspect, the present disclosure provides a system comprising: any of the herein-described computer products; and one or more processors for executing instructions stored on the computer readable medium.

In another aspect, the present disclosure provides a system comprising means for performing any of the herein-described methods.

In another aspect, the present disclosure provides a system comprising one or more processors configured to perform any of the herein-described methods.

In another aspect, the present disclosure provides a system comprising modules that respectively perform the steps of any of the herein-described methods.

A better understanding of the nature and advantages of embodiments of the present disclosure may be gained with reference to the following detailed description and the accompanying drawings.

### BRIEF DESCRIPTION OF THE DRAWINGS

FIG. 1: Further filtering of the 328-mRNA signature. The mean and standard deviation of log 2 FPKM (Fragments Per Kilobase Million) of all 12,678 genes in a RNASeq study GSE156063 (grey) and 328 biomarkers from meta-analysis (coral) are plotted in x-axis and y-axis respectively. Genes with mean log 2 FPKM and SD log 2FPKM≥1 (88 genes) were selected as gene signature for assay development.

FIGS. 2A-2E: AUC distribution for various mRNAs. AUROC were calculated to evaluate the performance of predicting infected vs uninfected samples from the 6 studies. FIG. 2A: Background AUC distribution using each of 12,065 mRNAs detected across all 6 studies. FIG. 2B: AUC distribution using each of 80 up-regulated or (FIG. 2C) 8 down-regulated mRNAs selected by absolute effect size≥0.6, FDR value≤0.1, and abundance and variance filtering in FIG. 1. FIG. 2D: AUC distribution for all 2-mRNA combinations from 88 biomarker mRNAs. FIG. 2E: AUC distribution for 10,000 randomly selected 2-mRNA combinations from 12,567 genes presented in the 6 datasets.

FIGS. 3A-3B. Geometric mean score of final 88 mRNAs distinguishes infected from uninfected samples in all 6 datasets. Geometric mean score is calculated as a scaled difference between the geometric means of expression of up-regulated (n=80) and down-regulated (n=8) mRNAs. FIG. 3A: Boxplot of geometric mean score in infected (Infection Class=1, grey triangles) vs uninfected (Infection Class=0, grey circles) shows separation between the two classes. FIG. 3B: Corresponding ROCs and summarized AUROCs derived based on the geometric means score in FIG. 3A.

FIGS. 4A-4B. Performance comparison between the signature set of mRNAs and the parsimonious set of mRNAs. ROC for individual datasets and summary (FIG. 4A) based on the final signature list of 88 mRNAs and (FIG. 4B) the parsimonious set of 5 mRNAs derived from the 88 mRNAs using forward search algorithm.

FIG. 5 illustrates a measurement system 500 according to an embodiment of the present disclosure.

FIG. 6 shows a block diagram of an example computer system usable with systems and methods according to embodiments of the present disclosure.

FIG. 7 illustrates the study design and group assignments for GSE163151.

FIGS. 8A-8C shows the performance of the 88-mRNA score in box plots for two groups (FIG. 8A), the ROC curve and AUC (FIG. 8B), and boxplots of scores for various types of viruses captured in the study of GSE163151 (FIG. 8C).

FIGS. 9A-9B shows the performance of our 88-mRNA score in box plots for two groups (FIG. 9A), ROC curve and AUC (FIG. 9B) for study dataset of GSE152075.

FIG. 10 shows the performance of our 88-mRNA score for COVID-19 patients with different viral loads and healthy controls.

FIGS. 11A-11B shows the performance of our 88-mRNA scores for COVID-19 patients divided by sex (FIG. 11A), and sex and viral load groups (FIG. 11B).

### TERMS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to primers, probes, oligonucleotides, template RNA or cDNA, genomic DNA, amplified subsequences of biomarker genes, or any polynucleotide composed of deoxyribonucleic acids (DNA), ribonucleic acids (RNA), or any other type of polynucleotide which is an N-glycoside of a purine or pyrimidine base, or modified purine or pyrimidine bases in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). “Nucleic acid”, “DNA” “polynucleotides, and similar terms also include nucleic acid analogs. The polynucleotides are not necessarily physically derived from any existing or natural sequence, but can be generated in any manner, including chemical synthesis, DNA replication, reverse transcription or a combination thereof.

“Primer” as used herein refers to an oligonucleotide, whether occurring naturally or produced synthetically, that is capable of acting as a point of initiation of synthesis when placed under conditions in which synthesis of a primer extension product which is complementary to a nucleic acid strand is induced i.e., in the presence of nucleotides and an agent for polymerization such as DNA polymerase and at a suitable temperature and buffer. Such conditions include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification such as a TaqMan real-time quantitative RT-PCR as described herein. The primers herein are selected to be substantially complementary to the different strands of each specific sequence to be amplified, and a given set of primers will act together to amplify a subsequence of the corresponding biomarker gene.

The term “gene” refers to the segment of DNA involved in producing a polypeptide chain. It can include regions preceding and following the coding region (leader and trailer) as well as intervening sequences (introns) between individual coding segments (exons).

SARS-COV-2 refers to the coronavirus that causes the infectious disease called COVID-19. The present methods can be used to determine presence or absence of a viral infection of any subject with any viral infection, and including any SARS-COV-2 infection, including by infection with viruses comprising the nucleotide sequences of, or comprising nucleotide sequences substantially identical (e.g., 70%, 75%, 80%, 85%, 90%, 95% or more identical) to all or a portion of GenBank reference numbers MN908947, LC757995, LC528232, and others. The methods can be performed with subjects having an infection detected by any method, and regardless of the presence or absence of symptoms.

“Respiratory sample” refers to a biological sample taken from any part of the respiratory tract, including the upper respiratory tract (nose, nasal cavity, pharynx) and lower respiratory tract (larynx, trachea, brochi, bronchioles, lungs) from a patient. For the purposes of the present methods, the sample comprises cells, e.g., epithelial cells, from the respiratory tract, thereby allowing detection and quantification of the biomarker mRNAs as described herein. A non-limiting list of suitable respiratory samples includes nasal swabs, interior nasal swab, mid-turbinate nasal swab, nasopharyngeal swab, oropharyngeal swab, saliva, sputum, oral swab, nasal aspirate or wash, bronchoalveolar lavage, washing, brushing, or aspirate, cough swab, endotracheal tip, tracheal aspirate, pleural aspirate, endotracheal aspirate, nasopharyngeal aspirate or secretion, and others.

As used herein, a “biomarker gene”, “biomarker mRNA”, or “biomarker” refers to a gene whose expression in cells of the respiratory tract (e.g., epithelial cells) is not only correlated with the presence or absence of a viral infection (also referred to as “viral infection status”), but also of a diagnostic value. The expression level of each of the genes need not be correlated with the viral infection status in all patients; rather, a correlation will exist at the population level, such that the level of expression is sufficiently correlated within the overall population of individuals with one or more symptoms of a respiratory infection and with a known viral infection status (i.e., infection or no infection) that it can be combined with the expression levels of other biomarker genes, in any of a number of ways, as described elsewhere herein, and used to calculate a biomarker or viral score. The values used for the measured expression level of the individual biomarker genes can be determined in any of a number of ways, including direct readouts from relevant instruments or assay systems, or values determined using methods including, but not limited to, forms of linear or non-linear transformation, rescaling, normalizing, z-scores, ratios against a common reference value, or any other means known to those of skill in the art. In some embodiments, the readout values of the biomarkers are compared to the readout value of a reference or control, e.g., a housekeeping gene whose expression is measured at the same time as the biomarkers. For example, the ratio or log ratio of the biomarkers to the reference gene can be determined. Preferred biomarker genes for the purposes of the present methods include IFITM1, TLNRD1, CDKN1C, INPP5E, and TSTD1, but others can be used as well, e.g., other biomarkers identified using the machine learning methods described herein, or other markers presented in Table 2 or Table 3, or the pairs of biomarkers presented in Table 4.

A “biomarker score” or “viral score”, terms which can be used interchangeably, refers to a value allowing a determination of the viral infection status (i.e., infected or uninfected) or the probability of a viral infection in a subject that is calculated from the measured expression levels of one or a plurality of biomarker genes, e.g., 1, 2, 3, 4, 5, 6, 7, 8, 9 10 or more individual biomarker genes, in respiratory cells (i.e., cells from the subject that are present in the respiratory tract, and/or that are present in the respiratory sample) from a subject. In some embodiments, the viral score is determined by applying a mathematical formula, or a series of mathematical formulae with specified interconnections, or a machine learning algorithm with optimized hyperparameters, or another parameter-based method by which the measured expression values of the biomarker genes can be used to generate a single “viral” score, including, e.g., arithmetic or geometric means with or without weights, linear regression, logistic regression, neural nets, or any other method known in the art. In particular embodiments, the “viral score” is used to determine the presence or absence of a respiratory viral infection in the subject, or the probability of a respiratory viral infection in the subject, by virtue of the score surpassing or not a given threshold value for the outcome in question, as described in more detail elsewhere herein. In some embodiments, the viral score is combined with other factors, such as the presence or severity of specific symptoms, patient factors (e.g. age, sex, vital signs, comorbidities), clinical risk scores (e.g., SOFA, qSOFA, APACHE score), epidemiological data regarding the prevalence of one or more viruses in the community, e.g., to improve the performance of the viral score in determining viral infection status.

The term “correlating” generally refers to determining a relationship between one random variable with another. In various embodiments, correlating a given biomarker level or score with the presence or absence of a condition or outcome (e.g., presence or absence of a respiratory viral infection) comprises determining the presence, absence or amount of at least one biomarker in a subject with the same outcome. In specific embodiments, a set of biomarker levels, absences or presences is correlated to a particular outcome, using receiver operating characteristic (ROC) curves.

“Conservatively modified variants” refers to nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants can have an increased stability, assembly, or activity.

As used in herein, the terms “identical” or percent “identity,” in the context of describing two or more polynucleotide sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. The identity can exist over a region that is at least about 10, 15, 20, 25, 30, 35, 40, 45, 50, or more nucleotides in length. In some embodiments, percent identity is determined over the full-length of the nucleic acid sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm with, e.g., the default parameters can be used. See, e.g., Altschul et al., (1990) J. Mol. Biol. 215: 403-410 and the National Center for Biotechnology Information website, ncbi.nlm.nih.gov.

### DETAILED DESCRIPTION

The present disclosure provides methods and compositions for detecting respiratory viral infections in nasal swab or other respiratory samples from subjects, and for determining effective treatment strategies for such subjects. The present methods and compositions involve biomarkers identified from the application of a machine learning workflow to respiratory viral infection training data, i.e., gene expression data from patients with known viral infections. Using these data, biomarkers have been identified that allow the generation of a viral score that can be used to indicate the presence or absence of a respiratory viral infection or the probability of a viral infection, e.g., in subjects with one or more symptoms of a respiratory infection, and/or in subjects at risk of developing a respiratory viral infection.

### I. Subjects

The present methods and compositions can be used to determine a viral score for subjects with one or more symptoms of a respiratory viral infection. In various embodiments, the subject may be an adult of any age, a child, or an adolescent. The subject may be male or female.

The subject has one or more symptoms of a respiratory infection. A non-limiting list of symptoms includes cough, sneezing, congestion in nasal sinuses or lungs, runny nose, sore throat, headache, body aches, shortness of breath, tight chest, wheezing, fever, fatigue, dizziness, feeling generally unwell, and others. The symptoms can also be present in any of various syndromes, including brochitis, bronchiolitis, pneumonia, croup, upper respiratory infection, asthma, pharyngoconjuctival fever, severe acute respiratory syndrome (SARS), and others. The symptoms can be mild, moderate, or severe. The present methods can be used to identify a respiratory viral infection in the subject, and thus to distinguish such subjects from others whose symptoms are caused by something other than a virus, e.g., a bacterial or fungal infection, or some other non-infectious condition. An indication of a viral infection using the present methods is not specific for any particular virus; the determination of the specific virus infecting the subject can then be determined, e.g., using nucleic acid amplification tests (NAATs).

In particular embodiments, the subject is present in a medical context, e.g., emergency care context (emergency room, urgent care facility), hospital, or any other clinical setting where diagnosis may take place. A clinical setting does not necessarily indicate that the patient is physically present in a hospital or clinical facility, however. For example, the patient may be at home but has provided a respiratory sample using an at-home testing kit, or at a local or drive-up testing facility. The results of the methods described herein can allow a determination of the optimal next step or plan of action for the subject's care. In some embodiments, a determination that the subject has a viral infection can indicate specific treatment such as anti-viral medications, additional testing to identify the specific virus causing the infection, and/or admittance to an ICU or other clinical facility, and/or administration of any of the treatments or procedures described herein. In some embodiments, a determination that the subject has a viral infection and subsequent or simultaneous identification of the infectious virus can indicate a specific treatment for the virus in question, admittance to the hospital, or in some cases discharge from the hospital or other clinical setting, e.g., if the identified virus is found to be non-life-threatening or relatively innocuous. In some embodiments, a determination that the subject does not have a viral infection can indicate, e.g., further testing for a bacterial infection that may warrant the administration of antibiotics, for a fungal infection, or for another non-infectious condition capable of causing the symptoms. In some cases, a negative result for a viral infection may indicate that the subject can be discharged from the hospital or emergency room, e.g., to return home for monitoring or to go to another, non-emergency ward.

In some embodiments, the subject is asymptomatic at the time of testing but is known to be at risk of or is suspected of having a viral infection, e.g., following close contact with an individual known to be infected. In such cases, the present methods can also be used to detect a viral infection in the subject, even though the subject is potentially presymptomatic. A negative result for a viral infection in such subjects may indicate that no infection has taken place, e.g. during the close contact, and that that the subject is therefore free of infection. A positive result would indicate a need for quarantine and/or follow-up testing.

The present methods can be used to detect any respiratory virus, e.g., influenza virus, coronavirus, SARS coronavirus, SARS COV or SARS-COV-2, MERS CoV, parainfluenza virus, respiratory syncytial virus (RSV), rhinovirus, metapneumovirus, coxsackie virus, echovirus, adenovirus, bocavirus, and others. In particular embodiments, the subject has a coronavirus, e.g., SARS-COV-2, or influenza. The subject can be infected during a pandemic, epidemic, seasonal, or isolated infection incident. In particular embodiments, the infection is detected in the context of an epidemic or pandemic, i.e., when health care resources are limited and rapid triage of subjects presenting in emergency care contexts is critical.

### II. Respiratory Samples

To assess the biomarker status of the patient, a respiratory sample is obtained from the subject. Suitable respiratory samples include nasal swabs, nasopharyngeal swab, oropharyngeal swab, saliva, sputum, oral swab, nasal aspirate or wash, bronchoalveolar lavage, washing, brushing, or aspirate, cough swab, endotracheal tip, tracheal aspirate, pleural aspirate, endotracheal aspirate, nasopharyngeal aspirate or secretion, and others. Generally, any sample that comprises cells, e.g., epithelial cells, from the subject's upper or lower respiratory tract and that allows detection and quantification of the herein-described mRNAs in the cells can be used. The respiratory sample can be obtained from the subject using conventional techniques known in the art. In some embodiments, the respiratory sample was originally obtained for direct testing of specific viruses (e.g., NAAT for SARS-COV-2 or influenza), and is subsequently (or simultaneously) tested more broadly for any viral infection using the present methods.

### III. Selection of Biomarkers

The presence of a respiratory viral infection in a subject is determined by calculating a score (“viral score” or “biomarker score”) based on the expression levels of biomarkers in a respiratory sample. In some embodiments, a panel of five biomarkers is used to calculate the score. In particular embodiments, the biomarker genes are IFITM1, TLNRD1, CDKN1C, INPP5E, and TSTD1. IFITM1 refers to interferon induced transmembrane protein 1 (see, e.g., NCBI gene ID 8519, the entire disclosure of which is herein incorporated by reference). TLNRD1 refers to talin rod domain containing 1 (see, e.g., NCBI gene ID 59274, the entire disclosure of which is herein incorporated by reference). CDKN1C refers to cyclin dependent kinase inhibitor 1C (see, e.g., NCBI gene ID 1028, the entire disclosure of which is herein incorporated by reference). INPP5E refers to inositol polyphosphate-5-phosphatase E (see, e.g., NCBI gene ID 56623, the entire disclosure of which is herein incorporated by reference), and TSTD1 refers to thiosulfate sulfurtransferase like domain containing 1 (see, e.g., NCBI gene ID 100131187, the entire disclosure of which is herein incorporated by reference).

However, other biomarkers can be used, e.g., in place of or in addition to any one or more of IFITM1, TLNRD1, CDKN1C, INPP5E, and TSTD1. For example, in some embodiments, biomarkers used in the methods include, but are not limited to, any one or more of the 328 biomarkers listed in Table 2. The biomarkers of Table 2 are GNLY, HAVCR2, MS4A6A, CD163, TLNRD1, GLRX, LHFPL2, MSR1, TPP1, ITPRIPL2, GIMAP1, ITGB2, C1orf162, FAM20A, FZD2, SLC39A8, GPBAR1, ENG, STABI, TRIM38, CCL18, SDS, GIMAP5, CSFIR, VAMP5, ADAP2, FLVCR2, GIMAP2, HLA-G, CAPG, CD247, FOXN2, EMILIN2, GIMAP8, MS4A7, FKBP5, CIQC, CD80, TRPV2, HK3, LPAR1, C1QA, MAP1S, SLAMF8, H4C8, CKAP4, PHF11, AIP, SLC16A3, STXBP2, GTPBP2, CYBB, GIMAP4, DUSP3, GZMH, RUBCN, CDKN1C, MFSD13A, NCOA7, HLA-B, SCARB2, LRRC8C, NKG7, STAT4, SH2DIA, ITGA5, CIQB, NAGK, MYEOV, SLFN12, AOAH, NOD1, OLR1, MAD2L2, RNASE2, DEFB1, CMKLR1, SLC4A2, VASH1, UBE2F, TNS3, TSPAN14, GAL3ST4, SLC1A3, OAS1, NCKAP1L, IFITM1, C6orf47, MGAT1, FCGR1A, SERPINB9P1, TMSB10, TIMP1, IL2RG, SDSL, RETN, SERTAD1, GZMK, MS4A4A, TMEM176B, HEG1, GZMB, PLOD1, RENBP, ELMO2, OLFML2B, FAM225A, CTSL, CD5, MTHFD2, HLA-A, CD33, MAFB, PRF1, SMCO4, CD2, TAP1, ATF4, RRAS, SAMD9, CD7, MILR1, IFITM3, DOK2, LY6E, GIMAP7, TMEM92, OSCAR, LGALS1, IFI6, TNFAIP8L2, FCGR1B, RASSF4, SQOR, NADK, TYMP, NOCT, TICAM1, ASPHD2, DESI1, SHISA5, NT5C3A, FPR3, MFSD12, SIGLEC10, FBX06, TMEM199, STOM, GCH1, FCN1, OASL, APBA3, CD300LF, IL10RA, P2RX4, GRN, FCER1G, TOR1B, IFITM2, MYO1G, OAS3, C2, CARD16, TRIM5, RIPK3, TENT5A, HLA-F, HERC5, ACODI, CD68, IRF7, LGALS9, C3AR1, LY96, SP100, IL32, BTN3A3, GZMA, TMUB2, ZBP1, POLR3D, FRMD3, PLA2G7, EPSTI1, IL6, SLCO2B1, HELZ2, DDX58, IFIT1, AIM2, ZC3HAV1, EMP3, KLF6, IFIT3, BATF2, NUCB1, ICAM2, LILRB4, XAF1, ISG15, OAS2, TMEM176A, DDX60, SERPING1, CST7, CCL8, NEXN, IFIT5, CD69, SAMD9L, IFI35, KCTD14, ABCD1, IFIT2, CMPK2, SOCS1, TNFSF13B, DDX60L, ZFYVE26, CIGALT1, DRAM1, HLA-E, DUSP6, IFIH1, BST2, MT2A, HESX1, IFNL2, GRAMD1B, APOBEC3G, ISG20, DTX3L, MX2, TLR7, IFI44L, IL15RA, TNFSF10, RSAD2, SECTM1, CCR1, SP110, COLGALT1, LAIR1, BATF, CCL2, IL27, CASP5, STAT2, PPPIR3D, CXCL10, GBP1, HAMP, MX1, GBPIP1, PARP12, HERC6, TMEM140, TFEC, EDEM2, GIMAP6, SIGLEC1, CALHM6, PARP9, IFI44, TRIM21, ATF5, TRIM22, CD48, USP18, KLHDC7B, RTP4, RBCK1, PARP14, APOL6, SLAMF7, GBP3, PARP10, EIF2AK2, ETV7, PIK3AP1, CASP1, TDRD7, SHFL, EIF3L, IK, NOA1, RPL3, CLDN8, CCDC190, LOC730202, MPC2, EBNA1BP2, SMIM19, PRPF8, ALDH9A1, VDAC3, PPP4R3B, DUS4L, SGSM2, COQ3, PPPIR14C, EEF1G, KIF3B, ALDH3A1, LOC541473, TPRG1L, CCT6B, TSTD1, TMEM14B, ERCC1, PEBP1, CAT, QARS1, PNMA1, TOMM34, PARVA, DDX46, PRDX5, HACL1, DMKN, FAM174A, ANKRD6, COQ7, GSTA1, PER3, INPP5E, TRIM45, and HLF.

In some embodiments, biomarkers used in the methods include, but are not limited to, any one or more of the 88 biomarkers listed in Table 3. The biomarkers of Table 3 are MS4A6A, TLNRD1, CIQC, C1QA, H4C8, SLC16A3, STXBP2, CDKN1C, HLA-B, NKG7, OAS1, IFITM1, C6orf47, TMSB10, TIMP1, IL2RG, SERTAD1, CTSL, HLA-A, MAFB, TAP1, SAMD9, CD7, IFITM3, LY6E, LGALS1, IFI6, NADK, TYMP, SIGLEC10, TMEM199, FCER1G, TOR1B, IFITM2, OAS3, RIPK3, HLA-F, CD68, IRF7, TMUB2, HELZ2, IFIT1, KLF6, IFIT3, XAF1, ISG15, OAS2, IFIT5, SAMD9L, IFI35, IFIT2, SOCS1, HLA-E, DUSP6, BST2, MT2A, APOBEC3G, ISG20, MX2, IFI44L, TNFSF10, RSAD2, SECTM1, CCR1, STAT2, PPPIR3D, CXCL10, GBP1, MX1, PARP9, IFI44, ATF5, TRIM22, KLHDC7B, RTP4, PARP14, GBP3, EIF2AK2, CASP1, SHFL, CCDC190, ALDH3A1, TPRG1L, TSTD1, PNMA1, PRDX5, GSTA1, and INPP5E. In some embodiments, the biomarkers include all 88 biomarkers listed in Table 3, or any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, or 87 biomarkers listed in Table 3. In some embodiments, the biomarkers include any one or more pairs of biomarkers listed in Table 4. Any number of biomarkers can be assessed in the methods, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 95, 100, 200, 300, 400, 500, or more biomarkers. It will be appreciated that any one or more of the herein-disclosed biomarkers can be used in combination with any other biomarkers, i.e., as subsets of a broader panel.

The biomarkers used in the present methods correspond to genes whose expression levels in respiratory cells (i.e., cells from the subject present in a respiratory sample) from the subject correlate with the presence of a respiratory viral infection in the subject, e.g., influenza virus, coronavirus, SARS coronavirus, SARS COV or SARS-COV-2, MERS CoV, parainfluenza virus, respiratory syncytial virus (RSV), rhinovirus, metapneumovirus, coxsackie virus, echovirus, adenovirus, bocavirus, or another viral infection. The expression level of the individual biomarkers can be elevated or depressed in individuals with a respiratory viral infection relative to the level in individuals without a viral infection. What is important is that the expression level of the biomarker is positively or inversely correlated with infection or non-infection, allowing the determination of an overall score, e.g., a viral score, or biomarker score, that can be used to determine the presence or absence of a respiratory viral infection.

Additional biomarkers can be assessed and identified using any standard analysis method or metric, e.g., by analyzing data from samples taken from subjects with or without a diagnosis of a respiratory viral infection, as described in more detail elsewhere herein and as illustrated, e.g., in the Examples. In some methods, the types of viral infections of the training data include that of the subject, but this is not required. Suitable metrics and methods include Pearson correlation, Kendall rank correlation, Spearman rank correlation, t-test, other non-parametric measures, over-sampling of the viral infection group, under-sampling of the non-infection group, and others including linear regression, non-linear regression, random forest and other tree-based methods, artificial neural networks, etc. In one embodiment, the feature selection uses univariate ranking with the absolute value of the Pearson correlation between the gene expression and outcome as the ranking metric. In some embodiments, features (genes) are selected via greedy forward search optimized on training accuracy. In some embodiments, features (genes) are selected via greedy forward search optimized on Area Under Operator Receiver Characteristic.

In some embodiments, data from multiple sources is inputted to a multi-cohort analysis using appropriate software, e.g., the MetaIntegrator package. In some embodiments, effect size is calculated for each mRNA within a study between infected and non-infected controls, e.g., as Hedges' g. In some embodiments, the pooled or summary effect size across all of the datasets is then computed, e.g., using DerSimonian and Laird's random effects model. In some embodiments, the effect size is then summarized and p values across all mRNAs corrected for multiple testing, e.g., based on Benjamini-Hochberg false discovery rate (FDR). In some embodiments, the p-values across the studies are then combined, e.g., using Fisher's sum of logs method, and the log-sum of p values that each mRNA is up- or downregulated is computed, along with corresponding p values. In some embodiments, metaanalysis is performed, e.g., by performing leave one-study out (LOO) analysis by removing one dataset at a time. In some embodiments, a greedy forward search can be used to identify a parsimonious set of genes with the greatest discriminatory power to distinguish samples from infected vs. non-infected subjects.

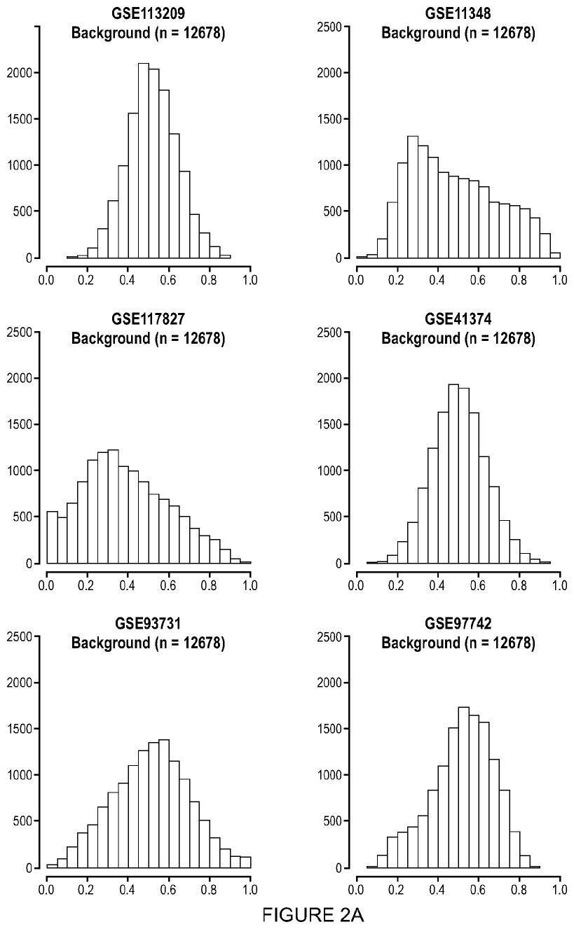

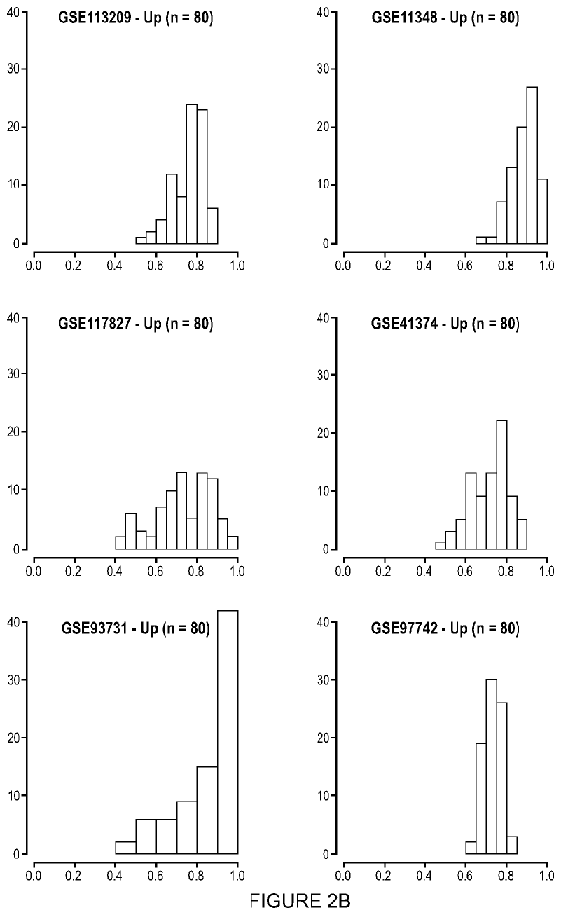

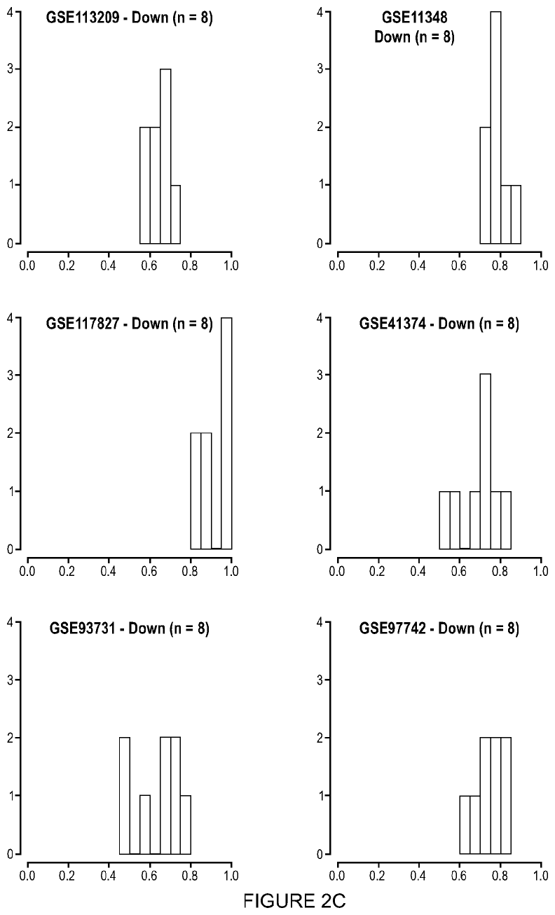

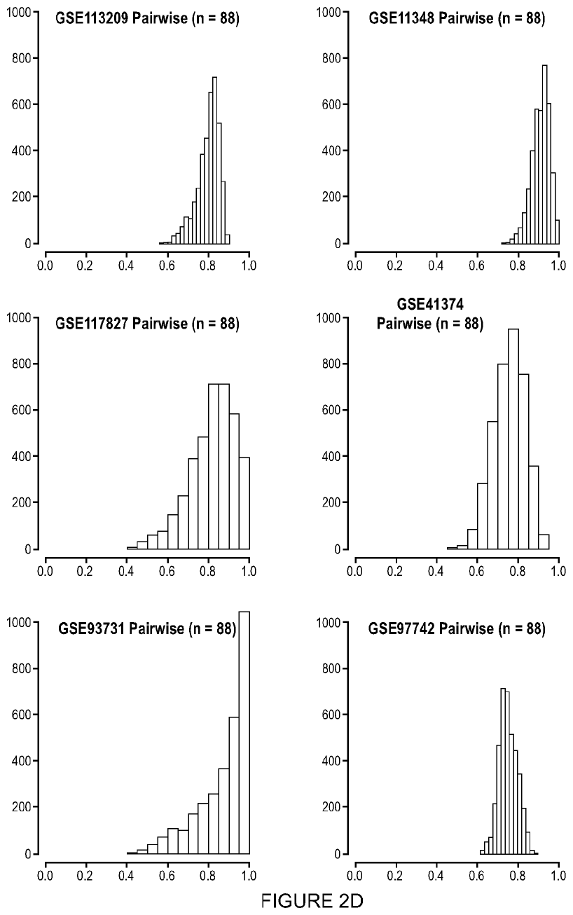

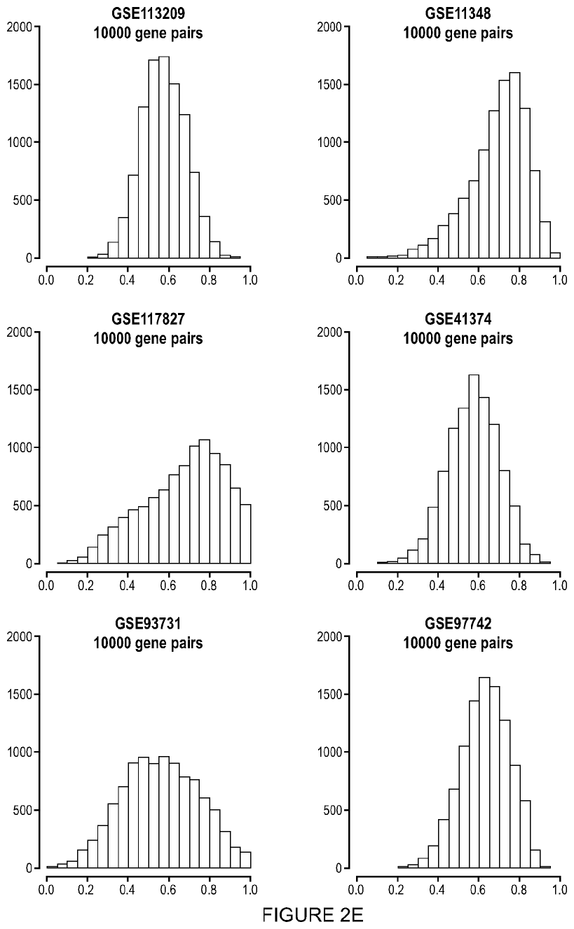

In particular embodiments, a machine learning workflow is applied to the training data, e.g., using a separate validation set or using cross-validation. For example, hyperparameter tuning can be used over a search space of parameters, e.g., parameters known to be effective for model optimization for infectious disease diagnosis. Examples of classifiers that can be used include linear classifiers such as Support Vector Machine with linear kernel, logistic regression, and multi-layer perceptron with linear activation function. Feature selection can be performed using the gene expression data for the candidate biomarkers as independent variables and using the known outcome as the dependent variable. The different models can be evaluated, e.g., using plots based on sensitivity and false-positive rates for each model, and the decision threshold evaluated during the hyperparameter search, and using ROC-like plots based on pooled cross-validated probabilities for the best models. (See, e.g., Ramkumar et al., Development of a Novel Proteomic Risk-Classifier for Prognostication of Patients with Early-Stage Hormone Receptor-Positive Breast Cancer. Biomarker Insights, Vol. 13, 1-9, 2018, FIG. 2A). Any of a number of different variants of cross-validation (CV) can be used, such as 5-fold random CV, 5-fold grouped CV, where each fold comprises multiple studies, and each study is assigned to exactly one CV fold, and leave-one-study-out (LOSO), where each study forms a CV fold. In some embodiments, the number of genes included in the final model can be limited, e.g., to 5, 6, 7, or 8, to facilitate translation to a rapid molecular assay. In some embodiments, other features such as overall expression level (e.g., genes with a mean and standard deviation of log 2FPKM that are both greater than 1) can be used to reduce the total number of genes.

### IV. Detecting Biomarker Expression

As described in more detail below, data sets corresponding to the biomarker gene expression levels as described herein are used to create a diagnostic or predictive rule or model based on the application of a statistical and machine learning algorithm, in order to produce a viral score. Such an algorithm uses relationships between a biomarker profile and an outcome, e.g., presence or absence of a viral infection (sometimes referred to as training data). The data are used to infer relationships that are then used to predict the status of a subject, e.g. the presence or absence of a respiratory viral infection.

The expression levels of the biomarkers can be assessed in any of a number of ways. In particular embodiments, the expression levels of the biomarkers are determined by measuring polynucleotide levels of the biomarkers. For example, once the respiratory sample has been collected and preserved, RNA can be extracted using any method, so long that it permits the preservation of the RNA for subsequent quantification of the expression levels of the biomarker genes and of any control genes to be used, e.g., housekeeping genes used as reference values for the biomarkers. RNA can be extracted, e.g., from preserved cells manually, or using a robotic apparatus, such as Qiacube (QIAGEN) with a commercial RNA extraction kit. In some embodiments, RNA extraction is not performed, e.g., for isothermal amplification methods. In such methods, expression levels can be determined directly through lysis of, e.g., epithelial cells, and then, e.g., reverse transcription and amplification of mRNA.

In some embodiments, the reference nucleic acid is a housekeeping gene or a product thereof, such as a corresponding mRNA transcript. In some embodiments, the reference nucleic acid includes an mRNA transcript that is a pre-mRNA molecule, a 5′ capped mRNA molecule, a 3′ adenylated mRNA molecule, or a mature mRNA molecule. In particular embodiments, the reference nucleic acid is a mature mRNA molecule obtained from a mammalian host that is also the source of the test sample. In some embodiments, the housekeeping gene or product thereof is expressed at a relatively constant rate by a cell of the host, such that the expression rate of the housekeeping gene can be used as a reference point against the expression of other host genes or gene products thereof. Suitable housekeeping genes are well known in the art and may include, e.g., GAPDH, ubiquitin, 18S (18S rRNA, e.g., HGNC (Human Genome Nomenclature Committee) nos. 44278-44281, 37657), ACTB (Actin beta, e.g., HGNC no. 132)), KPNA6 (Karyopherin subunit alpha 6, e.g., HGNC no. 6399), or RREB1 (ras-responsive element binding protein 1, e.g., HGNC no. 10449).

In some embodiments, the reference nucleic acid is a human housekeeping gene.

Exemplary human housekeeping genes suitable for use with the present methods include, but are not limited to, KPNA6, RREB1, YWHAB, Chromosome 1 open reading frame 43 (C1orf43), Charged multivesicular body protein 2A ((HMP2A), ER membrane protein complex subunit 7 (EMC7), Glucose-6-phosphate isomerase (GPI), Proteasome subunit, beta type, 2 (PSMB2), Proteasome subunit, beta type, 4 (PSMB+), Member RAS oncogene family (RAB7A), Receptor accessory protein 5 (REEP5), small nuclear ribonucleoprotein D3 (SNRPD)3), Valosin containing protein (VCP) and vacuolar protein sorting 29 homolog (VPS29). In some embodiments, any housekeeping gene provided at www/tau/ac/il˜elieis/HKG/ may be used (see, Eisenberg and Levanon., Trends Genet. (2013), 10:569-74).

The levels of transcripts of the biomarker genes, or their levels relative to one another, and/or their levels relative to a reference gene such as a housekeeping gene, can be determined from the amount of mRNA, or polynucleotides derived therefrom, present in a biological sample. Polynucleotides can be detected and quantified by a variety of methods including, but not limited to, NanoString (e.g., nCounter analysis), microarray analysis, polymerase chain reaction (PCR) (e.g., quantitative PCR (qPCR), droplet digital PCR (ddPCR), reverse transcriptase polymerase chain reaction (RT-PCR), quantitative RT-PCR (qRT-PCR)), isothermal amplification (e.g., loop-mediated isothermal amplification (LAMP), reverse transcription LAMP (RT-LAMP), quantitative RT-LAMP (qRT-LAMP)), RPA amplification, ligase chain reaction, branched DNA amplification, nucleic acid sequence-based amplification (NASBA), strand displacement assay (SDA), transcription-mediated amplification, rolling circle amplification (RCA), helicase-dependent amplification (HDA), single primer isothermal amplification (SPIA), nicking and extension amplification reaction (NEAR), transcription mediated assay (TMA), CRISPR-Cas detection, direct hybridization without amplification onto a functionalized surface (e.g., graphene biosensor), serial analysis of gene expression (SAGE), internal DNA detection switch, northern blotting, RNA fingerprinting, sequencing methods, Qbeta replicase, strand displacement amplification, transcription based amplification systems, nuclease protection (Si nuclease or RNAse protection assays), as well as methods disclosed in International Publication Nos. WO 88/10315 and WO 89/06700, and International Applications Nos. PCT/US87/00880 and PCT/US89/01025; herein incorporated by reference in their entireties, and methods using MacMan probes, flip probes, and TaqMan probes (see, e.g., Murray et al. (2014) J. Mol Diag. 16:6, pp 627-638). See, e.g., Draghici, Data Analysis Tools for DNA Microarrays, Chapman and Hall/CRC, 2003; Simon et al., Design and Analysis of DNA Microarray Investigations, Springer, 2004; Real-Time PCR: Current Technology and Applications, Logan, Edwards, and Saunders eds., Caister Academic Press, 2009; Bustin, A-Z of Quantitative PCR (IUL Biotechnology, No. 5), International University Line, 2004; Velculescu et al. (1995) Science 270: 484-487; Matsumura et al. (2005) Cell. Microbiol. 7: 11-18; Serial Analysis of Gene Expression (SAGE): Methods and Protocols (Methods in Molecular Biology), Humana Press, 2008; each of which is herein incorporated by reference in its entirety.

In some embodiments, the biomarker gene expression is detected using a gene expression panel such as a NanoString nCounter, which allows the quantification of biomarker gene expression without the need for amplification or cDNA conversion. In such methods, RNA obtained from the blood or other biological sample from the subject is hybridized in solution to probes, e.g., a labeled reporter probe and a capture probe for each biomarker and control sequence. The target RNA-probe complexes are then purified and immobilized on a solid support, and then quantified, with each marker-specific probe having a specific fluorescent signature that allows the quantification of the specific marker. Such methods and the generation of probes, e.g., capture probes and reporter probes, for such applications are known in the art and are described, e.g., on the website nanostring.com.

For amplification-based methods such as qRT-PCR or qRT-LAMP, the primers can be obtained in any of a number of ways. For example, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

Computer programs that are well known in the art are useful in the design of primers with the required specificity and optimal amplification properties, such as Oligo version 5.0 (National Biosciences). PCR methods are well known in the art, and are described, for example, in Innis et al., eds., PCR Protocols: A Guide To Methods And Applications, Academic Press Inc., San Diego, Calif. (1990); herein incorporated by reference in its entirety.

In some embodiments, microarrays are used to measure the levels of biomarkers. An advantage of microarray analysis is that the expression of each of the biomarkers can be measured simultaneously, and microarrays can be specifically designed to provide a diagnostic expression profile for a particular disease or condition (e.g., influenza, SARS-COV-2, etc.). Microarrays are prepared by selecting probes which comprise a polynucleotide sequence, and then immobilizing such probes to a solid support or surface. For example, the microarray may comprise a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site. Conditions for preparing microarrays, for hybridization conditions, and for detection of bound probes are well known in the art (see, e.g., Sambrook, et al., Molecular Cloning: A Laboratory Manual (3rd Edition, 2001); Ausubel et al., Current Protocols In Molecular Biology, vol. 2, Current Protocols Publishing, New York (1994); Shalon et al., 1996, Genome Research 6:639-645; Schena et al., Genome Res. 6:639-645 (1996); and Ferguson et al., Nature Biotech. 14:1681-1684 (1996)).

As noted above, the “probe” to which a particular polynucleotide molecule specifically hybridizes contains a complementary polynucleotide sequence. The probes of the microarray typically consist of nucleotide sequences of, e.g., no more than 1,000 nucleotides, or of 10 to 1,000 nucleotides or 10-200, 10-30, 10-40, 20-50, 40-80, 50-150, or 80-120 nucleotides in length. The probes may comprise DNA sequences, RNA sequences, or copolymer sequences of DNA and RNA. The polynucleotide sequences of the probes may also comprise DNA and/or RNA analogs, derivatives, or combinations thereof. For example, the probes can be modified at the base moiety, at the sugar moiety, or at the phosphate backbone (e.g., phosphorothioates). The polynucleotide sequences of the probes may be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes are preferably selected using an algorithm that takes into account binding energies, base composition, sequence complexity, cross-hybridization binding energies, and secondary structure. See Friend et al., International Patent Publication WO 01/05935, published Jan. 25, 2001; Hughes et al., Nat. Biotech. 19:342-7 (2001). An array will include both positive control probes, e.g., probes known to be complementary and hybridizable to sequences in the target polynucleotide molecules, and negative control probes, e.g., probes known to not be complementary and hybridizable to sequences in the target polynucleotide molecules. In addition, the present methods will include probes to both the biomarkers themselves, as well as to internal control sequences such as housekeeping genes, as described in more detail elsewhere herein.

In one embodiment, the disclosure provides a microarray comprising an oligonucleotide that hybridizes to an IFITM1 polynucleotide, an oligonucleotide that hybridizes to a TLNRD1 polynucleotide, an oligonucleotide that hybridizes to a CDKN1C polynucleotide, an oligonucleotide that hybridizes to an INPP5E polynucleotide, and an oligonucleotide that hybridizes to a TSTD1 polynucleotide. In some embodiments, the disclosure includes a microarray comprising an oligonucleotide that hybridizes to any of the biomarker genes listed in Table 2 or Table 3. In some embodiments, the disclosure includes a microarray comprising two oligonucleotides that hybridize to any of the biomarker pairs listed in Table 4.

In some embodiments, RNA sequencing (RNA-seq) can be used to measure the expression levels of biomarkers. RNA-seq is a technique based on enumeration of RNA transcripts using next-generation sequencing methodologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. Any high-throughput sequencing technology can be used for RNA-Seq, such as the Illumina IG, Applied Biosystems SOLID, and Roche 454 Life Science systems. The Helicos Biosciences tSMS system has the added advantage of avoiding amplification of target cDNA. Following sequencing, the resulting reads are either aligned to a reference genome or reference transcripts, or assembled de novo without the genomic sequence to produce a genome-scale transcription map that consists of both the transcriptional structure and/or level of expression for each gene.

In some embodiments, quantitative reverse transcriptase PCR (qRT-PCR) is used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

In some embodiments, the PCR employs the Taq DNA polymerase, which has a 5′-3′ nuclease activity but lacks a 3′-5′ proofreading endonuclease activity. TAQMAN PCR typically utilizes the 5′-nuclease activity of Taq or Tth polymerase to hydrolyze a hybridization probe bound to its target amplicon, but any enzyme with equivalent 5′ nuclease activity can be used. In such methods, two oligonucleotide primers are used to generate an amplicon typical of a PCR reaction, and a third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs that can be used to normalize patterns of gene expression include mRNAs for the housekeeping genes glyceraldehyde-3-phosphate-dehydrogenase (GAPDH) and beta-actin.

In particular embodiments, the biomarker gene expression is determined using isothermal amplification. Isothermal amplification is a process in which a target nucleic acid is amplified using a constant, single, amplification temperature (e.g., from about 30° C. to about 95 ºC). Unlike standard PCR, an isothermal amplification reaction does not include multiple cycles of denaturation, hybridization, and extension, of an annealed oligonucleotide to form a population of amplified target nucleic molecules (i.e., amplicons). There are various types of isothermal application known in the art, including but not limited to, loop-mediated isothermal amplification (LAMP), nucleic acid sequence based amplification NASBA, recombinase polymerase amplification (RPA), rolling circle amplification (RCA), nicking enzyme amplification reaction (NEAR), and helicase dependent amplification (HDA).

In particular embodiments, the isothermal amplification is real-time quantitative isothermal amplification, in which a target nucleic acid is amplified at a constant temperature and the target nucleic acid rate of amplification is monitored by fluorescence, turbidity, or similar measures (e.g., NEAR or LAMP). In some cases, RNA (e.g., mRNA) is isolated from a biological sample and is used as a template to synthesize cDNA by reverse-transcription. cDNA molecules are amplified under isothermal amplification conditions such that the production of amplified target nucleic acid can be detected and quantitated.

In particular embodiments, the isothermal amplification is Loop-Mediated Isothermal Amplification (LAMP). LAMP offers selectivity and employs a polymerase and a set of specially designed primers that recognize distinct sequences in the target nucleic acid (see, e.g., Nixon et al., (2014) Bimolecular Detection and Quantitation, 2:4-10; Schuler et al., (2016) Anal Methods., 8:2750-2755; and Schoepp et al., (2017) Sci. Transl. Med., 9:eaa13693). Unlike PCR, the target nucleic acid is amplified at a constant temperature (e.g., 60-65° C.) using multiple inner and outer primers and a polymerase having strand displacement activity. In some instances, an inner primer pair containing a nucleic acid sequence complementary to a portion of the sense and antisense strands of the target nucleic acid initiate LAMP. Following strand displacement synthesis by the inner primers, strand displacement synthesis primed by an outer primer pair can cause release of a single-stranded amplicon. The single-stranded amplicon may serve as a template for further synthesis primed by a second inner and second outer primer that hybridize to the other end of the target nucleic acid and produce a stem-loop nucleic acid structure. In subsequent LAMP cycling, one inner primer hybridizes to the loop on the product and initiates displacement and target nucleic acid synthesis, yielding the original stem-loop product and a new stem-loop product with a stem twice as long. Additionally, the 3′ terminus of an amplicon loop structure serves as initiation site for self-templating strand synthesis, yielding a hairpin-like amplicon that forms an additional loop structure to prime subsequent rounds of self-templated amplification. The amplification continues with accumulation of many copies of the target nucleic acid. The final products of the LAMP process are stem-loop nucleic acids with concatenated repeats of the target nucleic acid in cauliflower-like structures with multiple loops formed by annealing between alternately inverted repeats of a target nucleic acid sequence in the same strand.

In some embodiments, the isothermal amplification assay comprises a digital reverse-transcription loop-mediated isothermal amplification (dRT-LAMP) reaction for quantifying the target nucleic acid (see, e.g., Khorosheva et al., (2016) Nucleic Acid Research, 44:2 e10). Typically, LAMP assays produce a detectable signal (e.g., fluorescence) during the amplification reaction. In some embodiments, fluorescence can be detected and quantified. Any suitable method for detecting and quantifying florescence can be used. In some instances, a device such as Applied Biosystem's QuantStudio can be used to detect and quantify fluorescence from the isothermal amplification assay.

Any suitable method for detecting amplification of a target nucleic acid in a test sample by quantitative real-time isothermal amplification may be used to practice the present methods. In some embodiments, quantitative real-time isothermal amplification of a target nucleic acid in a test sample is determined by detecting of one or more different (distinct) fluorescent labels attached to nucleotides or nucleotide analogs incorporated during isothermal amplification of the target nucleic acid (e.g., 5-FAM (522 nm), ROX (608 nm), FITC (518 nm) and Nile Red (628 nm). In another embodiment, quantitative real-time isothermal amplification of a target nucleic acid in a test sample can be determined by detection of a single fluorophore species (e.g., ROX (608 nm)) attached to nucleotides or nucleotide analogs incorporated during isothermal amplification of the target nucleic acid. In some embodiments, each fluorophore species used emits a fluorescent signal that is distinct from any other fluorophore species, such that each fluorophore can be readily detected among other fluorophore species present in the assay.

In some embodiments, methods of detecting amplification of a target nucleic acid in a test sample by quantitative real-time isothermal amplification can include using intercalating fluorescent dyes, such as SYTO dyes (SYTO 9 or SYTO 82). In some embodiments, methods of detecting amplification of a target nucleic acid in a test sample by quantitative real-time isothermal amplification can include using unlabeled primers to isothermally amplify the target nucleic acid in the test sample, and a labeled probe (e.g., having a fluorophore) to detect isothermal amplification of the target nucleic acid in the test sample. In some embodiments, unlabeled primers are used to isothermally amplify a target nucleic acid present in the test sample, and a probe is used having a 5-FAM dye label on the 5′ end and a minor groove binder (MGB) and non-fluorescent quencher on the 3′ end to detect isothermal amplification of the target nucleic acid (e.g., TaqMan Gene Expression Assays from ThermoFisher Scientific).

In some embodiments, detecting amplification of the target nucleic acid in the test sample is performed using a one-step, or two-step, quantitative real-time isothermal amplification assay. In a one-step quantitative real-time isothermal amplification assay, reverse transcription is combined with quantitative isothermal amplification to form a single quantitative real-time isothermal amplification assay. A one-step assay reduces the number of hands-on manipulations as well as the total time to process a test sample. A two-step assay comprises a first-step, where reverse transcription is performed, followed by a second-step, where quantitative isothermal amplification is performed. It is within the scope of the skilled artisan to determine whether a one-step or two-step assay should be performed.

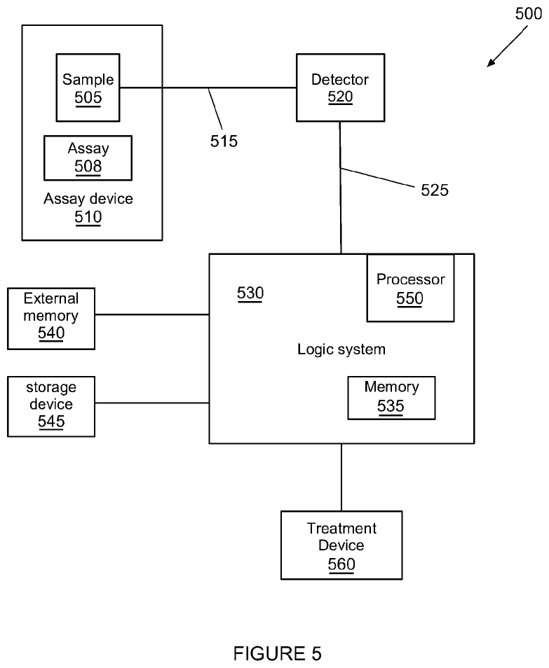

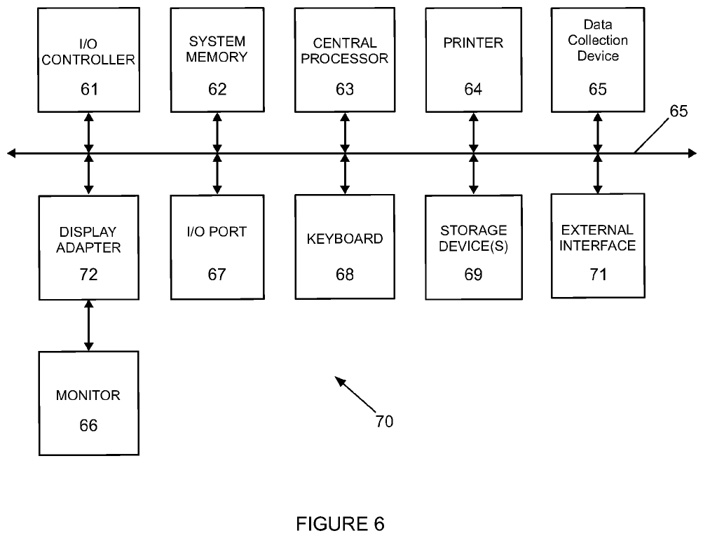

In some embodiments, the amplification and/or detection is carried out in whole or in part using an integrated measurement system, as illustrated in FIG. 5, which may also comprise a computer system as described elsewhere herein (see, e.g., FIG. 6).

In some embodiments, viral or biomarker scores are calculated based on the Tt (time to threshold) values for each of the tested biomarkers. This may be accomplished by, e.g., establishing standard curves for the isothermal or other amplification of the target nucleic acid (e.g., biomarker) and the reference nucleic acid (e.g., housekeeping gene). The standard curves can be obtained by performing real-time isothermal amplification assays using quantitated calibrator samples with multiple known input concentrations. Appropriate methods are provided in, e.g., PCT Publication No. WO 2020/061217, the entire disclosure of which is herein incorporated by reference.

For example, in some embodiments, to generate a standard curve, quantitated calibrator samples are obtained by performing serial dilutions of a quantitated material. For example, a template is serially diluted in a buffer at 10-fold concentration intervals yielding templates covering a range of concentrations from, e.g., approximately 109 copies/μL to approximately 102 copies/μL. The precise concentration of each calibrator sample can be determined using methods known in the art.

To obtain a standard curve, a real-time amplification assay is performed for each aliquot with a known quantity (e.g., 1 μL) of a respective calibrator sample with a respective concentration of the target nucleic acid. In a real-time amplification assay for each respective calibrator sample, the intensity of the fluorescence emitted by intercalating fluorescent dyes (e.g., dsDNA dyes) or fluorescent labels for the target nucleic acid is measured as a function of time. For example, a plot can be generated of fluorescence intensity as a function of time in a real-time quantitative amplification assay. A dashed line can be used to represent a pre-determined threshold intensity, and the elapsed time from the moment when the amplification is started is the time-to-threshold Tt. A respective time-to-threshold value can be determined from each respective fluorescence curve as a function of time. Thus, time-to-threshold values Ttₙ, Ttₙ₊₁, Ttₙ₊₂, etc., are obtained for the different calibrator samples.

For exponential amplifications, the time-to-threshold is linearly proportional to the logarithm (e.g., logarithm to base 10) of the starting copy number (also referred to as template abundance). A scatter plot of data points can be generated from the fluorescence curves. Each data point represents a data pair [Log₁₀(CopyNumber), Tt] (note that CopyNumber refers to starting number of copies of a nucleic acid in an amplification assay). In some embodiments, the data points fall approximately on a straight line. A linear regression is then performed on the data points in the plot to obtain the straight line that best fits the data points with the least amount of total deviations. The result of the linear regression is a straight line represented by the following equation,

$$Tt = m \times {Log}_{10} ( CopyNumber ) + b , \qquad ( 1 )$$

where m is the slope of the line, and b is y-intercept. The slope m represents the efficiency of the isothermal amplification of the target nucleic acid; b represents a time-to-threshold as template copy number approaches zero. The straight line represented by Equation (1) is referred to as the standard curve.

In some embodiments, replicates (e.g., triplicates) of isothermal amplification assays may be run for each sample in order to gain a higher level of confidence in the data. Replicate time-to-threshold values can be averaged, and standard deviations can be calculated.

Once the standard curve is established for a given isothermal amplification assay, the standard curve can be used to convert a time-to-threshold value to a starting copy number for future runs of the amplification assay of unknown starting numbers of copies of the target nucleic acid, using the following equation,

$$CopyNumber = {10}^{\frac{T t - b}{m}} . \qquad ( 2 )$$

Normally, the data points for low copy numbers or very high copy numbers may fall off of the straight line. The range of copy numbers within which the data points can be represented by the straight line is referred to as the dynamic range of the standard curve. The linear relationship between the time-to-threshold and the logarithmic of copy number represented by the standard curve would be valid only within the dynamic range.

If the amplification efficiencies for a target nucleic acid and a reference nucleic acid are different for a given isothermal amplification assay, it may be necessary to obtain separate standard curves for the target nucleic acid and the reference nucleic acid. Thus, two sets of real-time isothermal amplification assays may be performed, one set for establishing the standard curve for the target nucleic acid, the other set for establishing the standard curve for the reference nucleic acid. In cases where multiple target nucleic acids are considered (e.g., for a panel of five biomarkers as described herein), a standard curve for each target nucleic acid may be obtained.

In some embodiments, the standard curves are generated prior to obtaining a test sample. That is, the standard curves are not generated on-board with the quantitative isothermal amplification of the test sample. Such standard curves may be referred to as off-board standard curves. Off-board standard curves may be used for estimating relative abundance values. For example, for a test sample of unknown input concentration of a target nucleic acid, a first real-time amplification assay is performed for a first aliquot of the test sample to obtain a first time-to-threshold value with respect to the target nucleic acid. A second real-time isothermal amplification assay is then performed for a second aliquot of the test sample to obtain a second time-to-threshold value with respect to a reference nucleic acid. The first aliquot and the second aliquot contain substantially the same amount of the test sample. The first time-to-threshold value may then be converted into starting number of copies of the target nucleic acid using the standard curve of the target nucleic acid. Similarly, the second time-to-threshold value may be converted into starting number of copies of the reference nucleic acid using the standard curve of the reference nucleic. The starting number of copies of the target nucleic acid is then normalized against that of the reference nucleic acid to obtain a relative abundance value.

In cases where the amplification efficiencies for a target nucleic acid and a reference nucleic acid have approximately the same value that is known, relative abundance may be obtained directly from time-to-threshold values without using standard curves.

### V. Calculating Biomarker (or Viral) Scores

To determine the likelihood of a viral infection, a model (e.g., the model with the hyperparameter configuration providing the maximum AUC) is applied to the biomarker expression data from the subject to determine a score, e.g., a “viral score” or “biomarker score”, that is indicative of the probability of a viral infection. This score can be used, e.g., to classify the subject into any of a number of bins, e.g., 2 bins corresponding to the probable presence or absence of a viral infection, or 3 bins with a “low”, “intermediate” or “indeterminate”, and “high” likelihood of a viral infection. In a particular embodiment, the model uses logistic regression and the selected biomarker genes, e.g., IFITM1, TLNRD1, CDKN1C, INPP5E, and TSTD1, to calculate the score. The probability of a viral infection as determined using the model is then used to determine the optimal treatment of the subject, as described in more detail elsewhere herein.

The viral or biomarker score can be calculated, e.g., by taking the sum, product, or quotient of the gene expression levels (as used herein, “gene levels”, “expression levels”, and “gene expression levels” are interchangeable), taken in terms of their absolute levels or their relative levels as compared to control genes, e.g., housekeeping genes, or by inputting them into a linear or nonlinear algorithm that incorporates at least the measured gene levels, e.g., the measured levels of 2, 3, 4, 5, 6, 7, 8, 9, 10 or more biomarker genes, into an interpretable score. In a particular embodiment, the score is calculated based on the expression data obtained for a panel of five biomarkers.

In semi-quantitative methods, a threshold or cut-off value is suitably determined, and is optionally a predetermined value. In particular embodiments, the threshold value is predetermined in the sense that it is fixed, for example, based on previous experience with the assay and/or a population of subjects with a given outcome or outcomes, e.g., with a population of 50, 100, 150, 200, 250, 300, 350, 400, 450, 500, or more subjects with a viral infection or without a viral infection. Alternatively, the predetermined value can also indicate that the method of arriving at the threshold is predetermined or fixed even if the particular value varies among assays or can even be determined for every assay run.

For the statistical analyses described herein, e.g., for the selection of biomarkers to be included in the calculation of a score or in the calculation of a probability or likelihood of a particular viral infection status in a patient, as well as for diagnostic or therapeutic assessments made in view of a given viral or biomarker score, other relevant information can also be considered, such as clinical data regarding the symptoms presented by each individual. This can include demographic information such as age, race, and sex; information regarding a presence, absence, degree, stage, severity or progression of a condition, phenotypic information, such as details of phenotypic traits, genetic or genetically regulated information, amino acid or nucleotide related genomics information, results of other tests including imaging, biochemical and hematological assays, other physiological scores, or the like.

As described above, the abundance values for the individual biomarker genes in cells of the respiratory sample can be combined using a mathematical formula or a machine learning or other algorithm to produce a single diagnostic score, such as the viral score that can indicate the presence or absence (or probability) of a respiratory viral infection in a subject. In these embodiments, the produced score carries more predictive power than any individual gene level alone (e.g., has a greater area under the receiver-operating-characteristic curve for discrimination of infection or non-infection).

In some embodiments, types of algorithms for integrating multiple biomarkers into a single diagnostic score may include, but not limited to, a difference of geometric means, a difference of arithmetic means, a difference of sums, a simple sum, and the like. In some embodiments, a diagnostic score may be estimated based on the relative abundance values of multiple biomarkers using machine-learning models, such as a regression model, a tree-based machine-learning model, a support vector machine (SVM) model, an artificial neural network (ANN) model, or the like.

Biomarker data may also be analyzed by a variety of methods to determine the statistical significance of differences in observed levels of biomarkers between test and reference expression profiles in order to evaluate the viral infection status or probability of a viral infection in a subject. In certain embodiments, patient data is analyzed by one or more methods including, but not limited to, multivariate linear discriminant analysis (LDA), receiver operating characteristic (ROC) analysis, principal component analysis (PCA), ensemble data mining methods, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; Mclachlan (2004) Discriminant Analysis and Statistical Pattern Recognition. Wiley Interscience; Zweig et al. (1993) Clin. Chem. 39:561-577; Pepe (2003) The statistical evaluation of medical tests for classification and prediction, New York, N.Y.: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, Calif., USA; English et al. (2009) J. Biomed. Inform. 42(2):287-295; Zhang (2007) Bioinformatics 8: 230; Shen-Orr et al. (2010) Journal of Immunology 184:144-130; Qiu et al. (2011) Nat. Biotechnol. 29(10):886-891; Ru et al. (2006) J. Chromatogr. A. 1111(2):166-174, Jolliffe Principal Component Analysis (Springer Series in Statistics, 2.sup.nd edition, Springer, N Y, 2002), Koren et al. (2004) IEEE Trans Vis Comput Graph 10:459-470; herein incorporated by reference in their entireties.)

It is not necessary that all of the biomarkers are elevated or depressed relative to control levels in a respiratory sample from a given subject to give rise to a determination of a viral infection. For example, for a given biomarker level there can be some overlap between individuals falling into different probability categories. However, collectively the combined levels for all of the biomarker genes included in the assay will give rise to a score that, if it surpasses a threshold, e.g., a threshold derived from at least 50, 100, 150, 200, 250, 300, 350, 400, 500 or more patients with a respiratory viral infection, and/or of 10, 20, 30, 40, 50, 100, 150, 200, 250, 300, 350, 400, 500 or more control individuals without a respiratory viral infection, that allows a determination concerning the respiratory viral infection status of the subject. For example, for a determination of an absence of a respiratory viral infection, the threshold could be such that at across a population of at least 100 individuals with a respiratory viral infection and 100 patients without a respiratory viral infection, at least 90% of the subjects without a respiratory viral infection are above the threshold. It will be appreciated that in any given assay there can be more than one threshold, e.g., a threshold in one direction that indicates the presence of a respiratory viral infection, and a threshold in the other direction that indicates an absence of a respiratory viral infection. It will also be appreciated that an indication of a viral infection is not specific to the type of infection, as it can broadly detect any viral infection. Further, an indication of an absence of a viral infection is independent of the subject's overall infection status or other aspects of the subject's condition. For example, a subject with an indicated absence of a viral infection could still have, e.g., a bacterial or fungal infection, or could be free of any type of infection.

As used herein, the terms “probability,” and “risk” with respect to a given outcome refer to conditional probability that subjects with a particular score actually have the condition (e.g., viral infection) based on a given mathematical model. An increased probability or risk for example can be relative or absolute and can be expressed qualitatively or quantitatively. For instance, an increased risk can be expressed as simply determining the subject's score and placing the test subject in an “increased risk” category, based upon previous population studies. Alternatively, a numerical expression of the test subject's increased risk can be determined based upon an analysis of the biomarker or risk score.

In some embodiments, likelihood is assessed by comparing the level of a biomarker or viral score to one or more preselected or threshold levels. Threshold values can be selected that provide an acceptable ability to predict the presence or absence of a viral infection. In illustrative examples, receiver operating characteristic (ROC) curves are calculated by plotting the value of a biomarker or viral score in two populations in which a first population has a first condition (e.g., no viral infection) and a second population has a second condition (e.g., viral infection).

For any particular biomarker, a distribution of biomarker levels for subjects with and without a disease will likely overlap, and some overlap will be present for biomarker or viral scores as well. Under such conditions, a test does not absolutely distinguish a first condition and a second condition with 100% accuracy, and the area of overlap indicates where the test cannot distinguish the first condition and the second condition. A threshold value is selected, above which (or below which, depending on how a biomarker or viral score changes with a specified condition or prognosis) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143: 29-36 (1982)).

In some embodiments, a positive likelihood ratio, negative likelihood ratio, odds ratio, and/or AUC or receiver operating characteristic (ROC) values are used as a measure of a method's ability to predict the viral infection status. As used herein, the term “likelihood ratio” is the probability that a given test result would be observed in a subject with a condition or outcome of interest divided by the probability that that same result would be observed in a patient without the condition or outcome of interest. Thus, a positive likelihood ratio is the probability of a positive result observed in subjects with the specified condition or outcome divided by the probability of a positive results in subjects without the specified condition or outcome. A negative likelihood ratio is the probability of a negative result in subjects without the specified condition or outcome divided by the probability of a negative result in subjects with specified condition or outcome.

The term “odds ratio,” as used herein, refers to the ratio of the odds of an event occurring in one group (e.g., an absence of a viral infection) to the odds of it occurring in another group (e.g., a presence of a viral infection), or to a data-based estimate of that ratio. The term “area under the curve” or “AUC” refers to the area under the curve of a receiver operating characteristic (ROC) curve, both of which are well known in the art. AUC measures are useful for evaluating the accuracy of a classifier across the complete decision threshold range. Classifiers with a greater AUC have a greater capacity to classify unknowns correctly between two or more groups of interest (e.g., presence or absence of a viral infection, or a low, intermediate, or high probability of viral infection). ROC curves are useful for plotting the performance of a particular feature (e.g., any of the biomarker expression levels or biomarker scores described herein and/or any item of additional biomedical information) in distinguishing or discriminating between two populations (e.g., viral infection and no viral infection). Typically, the feature data across the entire population (e.g., the cases and controls) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls.

Although this refers to scenarios in which a feature is elevated in cases compared to controls, it also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to produce a single value, and this single value can be plotted in a ROC curve. Additionally, any combination of multiple features, in which the combination derives a single output value, can be plotted in a ROC curve. These combinations of features can comprise a test. The ROC curve is the plot of the sensitivity of a test against 1-specificity of the test, where sensitivity is traditionally presented on the vertical axis and 1-specificity is traditionally presented on the horizontal axis. Thus, “AUC ROC values” are equal to the probability that a classifier will rank a randomly chosen positive instance higher than a randomly chosen negative one.

In some embodiments, at least two (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 or more) biomarker genes are selected to discriminate between subjects with a first condition or outcome and subjects with a second condition or outcome with at least about 70%, 75%, 80%, 85%, 90%, 95% accuracy or having a C-statistic of at least about 0.70, 0.75, 0.80, 0.85, 0.90, 0.95.

In the case of a positive likelihood ratio, a value of 1 indicates that a positive result is equally likely among subjects in both the “condition” and “control” groups (e.g., in individuals with or without a viral infection); a value greater than 1 indicates that a positive result is more likely in the condition group (e.g., in individuals with a viral infection); and a value less than 1 indicates that a positive result is more likely in the control group (e.g., in individuals without a viral infection). In this context, “condition” is meant to refer to a group having one characteristic (e.g., viral infection) and “control” group lacking the same characteristic (e.g., no viral infection). In the case of a negative likelihood ratio, a value of 1 indicates that a negative result is equally likely among subjects in both the “condition” and “control” groups; a value greater than 1 indicates that a negative result is more likely in the “condition” group; and a value less than 1 indicates that a negative result is more likely in the “control” group.

In certain embodiments, the biomarker or viral score is calculated, based on the measured levels of the biomarkers in subjects with a viral infection or without a viral infection, such that the likelihood ratio corresponding to the high risk bin is 1.5, 2, 2.5, 3, 3.5, 4, or more, or that the likelihood ratio corresponding to the low risk bin is 0.15, 0.10, 0.05, or lower, for the presence of a viral infection.

In the case of an odds ratio, a value of 1 indicates that a positive result is equally likely among subjects in both the condition” and “control” groups; a value greater than 1 indicates that a positive result is more likely in the “condition” group; and a value less than 1 indicates that a positive result is more likely in the “control” group. In the case of an AUC ROC value, this is computed by numerical integration of the ROC curve. The range of this value can be 0.5 to 1.0. A value of 0.5 indicates that a classifier (e.g., a biomarker level) cannot discriminate between cases and controls (e.g., non-survivors and survivors), while 1.0 indicates perfect diagnostic accuracy. In certain embodiments, biomarker gene levels and/or biomarker scores are selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In certain embodiments, the biomarker gene levels and/or biomarker scores are selected to exhibit an odds ratio of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less. In certain embodiments, biomarker gene levels and/or biomarker scores are selected to exhibit an AUC ROC value of greater than 0.5, preferably at least 0.6, more preferably 0.7, still more preferably at least 0.8, even more preferably at least 0.9, and most preferably at least 0.95.

In some cases, multiple thresholds can be determined in so-called “tertile,” “quartile,” or “quintile” analyses. In these methods, the “diseased” and “control groups” (or “high risk” and “low risk”) groups are considered together as a single population, and are divided into 3, 4, or 5 (or more) “bins” having equal numbers of individuals. The boundary between two of these “bins” can be considered “thresholds.” A risk (of a particular diagnosis or prognosis for example) can be assigned based on which “bin” a test subject falls into. In some embodiments of the present methods, subjects are assigned to one of three bins, i.e. “low”, “intermediate”, or “high”, referring to the probability of a viral infection based on the viral scores obtained using the present methods. For example, subjects can be classified according to the estimated probability of a viral infection into 3 bins: low likelihood (bin 1), intermediate (bin 2), and high-likelihood (bin 3). The bins are defined, e.g., such that the likelihood ratios are <0.15 in bin 1, from 0.15 to 5 in bin 2, and >5 in bin 3.

The phrases “assessing the likelihood” and “determining the likelihood,” as used herein, refer to methods by which the skilled artisan can predict the presence or absence of a condition (e.g., respiratory viral infection) in a patient. The skilled artisan will understand that this phrase includes within its scope an increased probability that a condition is present or absent in a patient; that is, that a condition is more likely to be present or absent in a subject. For example, the probability that an individual identified as having a specified condition actually has the condition can be expressed as a “positive predictive value” or “PPV.” Positive predictive value can be calculated as the number of true positives divided by the sum of the true positives and false positives. PPV is determined by the characteristics of the predictive methods of the present methods as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition or outcome actually does not have that condition can be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined to have a significant probability of having or not having a specified condition or outcome. By “significant probability” is meant that the subject has a reasonable probability (0.6, 0.7, 0.8, 0.9 or more) of having, or not having, a specified condition or outcome.

In some embodiments, the biomarker score is combined with one or more clinical risk scores, such as SOFA, qSOFA, or APACHE. For example, a formula is used to combine (i) either the individual gene expression values or the output from a classifier that uses the gene expression values, with (ii) the clinical risk score, to generate (iii) a new score that is useful to the clinician.

### VI. Direct Detection of Virus in the Sample

In particular embodiments, in addition to determining the presence or absence of a respiratory viral infection based on the expression of host biomarkers as described herein, a direct test for one or more viruses is performed on the sample. For example, in some embodiments, a direct test for a virus, e.g., SARS-COV-2, influenza, coronavirus, SARS coronavirus, SARS COV, MERS CoV, parainfluenza virus, respiratory syncytial virus (RSV), rhinovirus, metapneumovirus, coxsackie virus, echovirus, adenovirus, bocavirus, or other, is performed. The test is performed using standard methods, such as viral culture, antigen detection, or nucleic acid amplification tests (NAATs). Any suitable NAAT can be used for the candidate virus in question. For example, in some embodiments the NAAT involves the polymerase chain reaction (PCR), reverse transcription polymerase chain reaction (RT-PCR), transcription mediated amplification (TMA), strand displacement amplification (SDA), or loop mediated isothermal amplification (LAMP) methods such as nicking endonuclease amplification reaction (NEAR), helicase-dependent amplification (HDA), or clustered regularly interspaced short palindromic repeats (CRISPR)-based methods.

In some cases, such tests allow the determination of the specific virus causing the infection, such that the results of the assays simultaneously demonstrate both the presence of a virus and the determination of the specific virus. In some cases, however, the methods as described herein indicate the presence of a viral infection, but the direct test for one or more specific viruses is negative. In such cases, the present methods allow a determination that the subject is infected with a virus other than those that have been tested for. This determination can then lead to testing for other viruses, and can also prevent the initiation of inappropriate treatments (such as antibiotic therapy for a presumed bacterial infection if a direct viral test is negative).

The different tests (i.e., a test using the present methods for the presence of any viral infection, and one or more direct tests for the presence of specific viruses) can be performed in any order, and using any sample, e.g., a respiratory sample originally obtained for direct detection of one or more specific viruses, a respiratory sample originally obtained for a broad viral test according to the present methods, a respiratory sample originally obtained for both direct detection of specific viruses and for a broad viral test according to the present methods, or a respiratory sample originally obtained for another purpose altogether.

### VII. Treatment Decisions

The methods described herein may be used to classify subjects according to the presence or absence of a respiratory viral infection, or the probability of a respiratory viral infection. In some embodiments, the subjects are classified as having or not having a respiratory viral infection. In some embodiments, subjects are classified as having high, low, or intermediate probability of having a viral infection. Subjects with a high probability of having a viral infection could receive further testing to identify the specific virus causing the infection. Such further testing can be performed simultaneously with the biomarker testing (e.g., both tested at substantially the same time using the same sample), or could be performed subsequently, e.g., using the same sample or using a later-obtained sample, following a positive biomarker test result. The identification of a viral infection can also indicate the delivery of medical care appropriate for the specific virus involved, such as an antiviral medication or other form of medical care, e.g., as described elsewhere herein. For example, in some embodiments, patients identified as having a life-threatening or otherwise severe viral infection by the methods described herein may be sent immediately to the ICU or other hospital ward or clinical facility for treatment. In some embodiments, patients identified as having a non-life threatening or relatively harmless viral infection may be discharged from the emergency room setting, e.g., released from the hospital for self-isolation and further monitoring and/or treated in a regular hospital ward or at home. As used herein, “medical care” comprises any action taken with respect to the treatment of the subject, whether in an emergency room or urgent care context, in another clinical facility or context, or at home, in order to alleviate, eliminate, slow the progression of, or in any way improve any aspect or symptom of the viral infection, including, but not limited to, administering a therapeutic drug, administering organ-supportive care, and admission to an ICU or other hospital ward or clinical facility.

Importantly, as noted above, in cases where a viral infection is detected using the present methods, a clinician can forgo unnecessarily administering a treatment for another infection, e.g., administering antibiotics for a bacterial infection, which might, in the absence of a positive biomarker test, be performed following a negative direct test, e.g., NAAT, for a specific virus.

In the case of severe, e.g., life-threatening viral infections, treatment of a patient may comprise constant monitoring of bodily functions and providing life support equipment and/or medications to restore normal bodily function. ICU treatment may include, for example, using mechanical ventilators to assist breathing, equipment for monitoring bodily functions (e.g., heart and pulse rate, air flow to the lungs, blood pressure and blood flow, central venous pressure, amount of oxygen in the blood, and body temperature), pacemakers, defibrillators, dialysis equipment, intravenous lines, bronchodilators, feeding tubes, suction pumps, drains, and/or catheters, and/or administering various drugs for treating the life threatening condition (e.g., sepsis, severe trauma, or burn). ICU treatment may further comprise administration of one or more analgesics to reduce pain, and/or sedatives to induce sleep or relieve anxiety, and/or barbiturates (e.g., pentobarbital or thiopental) to medically induce coma.

In certain embodiments, a patient diagnosed with a viral infection is further administered a therapeutically effective dose of an antiviral agent, such as a broad-spectrum antiviral agent, an antiviral vaccine, a neuraminidase inhibitor (e.g., zanamivir (Relenza) and oseltamivir (Tamiflu)), a nucleoside analog (e.g., acyclovir, zidovudine (AZT), and lamivudine), an antisense antiviral agent (e.g., phosphorothioate antisense antiviral agents (e.g., Fomivirsen (Vitravene) for cytomegalovirus retinitis), protease inhibitors, morpholino antisense antiviral agents), an inhibitor of viral uncoating (e.g., Amantadine and rimantadine for influenza, Pleconaril for rhinoviruses), an inhibitor of viral entry (e.g., Fuzeon for HIV), an inhibitor of viral assembly (e.g., Rifampicin), or an antiviral agent that stimulates the immune system (e.g., interferons). Exemplary antiviral agents include Abacavir, Aciclovir, Acyclovir, Adefovir, Amantadine, Amprenavir, Ampligen, Arbidol, Atazanavir, Atripla (fixed dose drug), Balavir, Cidofovir, Combivir (fixed dose drug), Dolutegravir, Darunavir, Delavirdine, Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine. Other drugs that may be administered include chloroquine, hydroxychloroquine, sarilumab, remdesivir, azithromycin, and statins.

In some embodiments, a critically ill patient diagnosed with a viral infection is further administered a therapeutically effective dose of an innate or adaptive immunity modulator such as abatacept, Abetimus, Abrilumab, adalimumab, Afelimomab, Aflibercept, Alefacept, anakinra, Andecaliximab, Anifrolumab, Anrukinzumab, Anti-lymphocyte globulin, Anti-thymocyte globulin, antifolate, Apolizumab, Apremilast, Aselizumab, Atezolizumab, Atorolimumab, Avelumab, azathioprine, Basiliximab, Belatacept, Belimumab, Benralizumab, Bertilimumab, Besilesomab, Bleselumab, Blisibimod, Brazikumab, Briakinumab, Brodalumab, Canakinumab, Carlumab, Cedelizumab, Certolizumab pegol, chloroquine, Clazakizumab, Clenoliximab, corticosteroids, cyclosporine, Daclizumab, Dupilumab, Durvalumab, Eculizumab, Efalizumab, Eldelumab, Elsilimomab, Emapalumab, Enokizumab, Epratuzumab, Erlizumab, etanercept, Etrolizumab, Everolimus, Fanolesomab, Faralimomab, Fezakinumab, Fletikumab, Fontolizumab, Fresolimumab, Galiximab, Gavilimomab, Gevokizumab, Gilvetmab, golimumab, Gomiliximab, Guselkumab, Gusperimus, hydroxychloroquine, Ibalizumab, Immunoglobulin E, Inebilizumab, infliximab, Inolimomab, Integrin, Interferon, Ipilimumab, Itolizumab, Ixekizumab, Keliximab, Lampalizumab, Lanadelumab, Lebrikizumab, leflunomide, Lemalesomab, Lenalidomide, Lenzilumab, Lerdelimumab, Letolizumab, Ligelizumab, Lirilumab, Lulizumab pegol, Lumiliximab, Maslimomab, Mavrilimumab, Mepolizumab, Metelimumab, methotrexate, minocycline, Mogamulizumab, Morolimumab, Muromonab-CD3, Mycophenolic acid, Namilumab, Natalizumab, Nerelimomab, Nivolumab, Obinutuzumab, Ocrelizumab, Odulimomab, Oleclumab, Olokizumab, Omalizumab, Otelixizumab, Oxelumab, Ozoralizumab, Pamrevlumab, Pascolizumab, Pateclizumab, PDE4 inhibitor, Pegsunercept, Pembrolizumab, Perakizumab, Pexelizumab, Pidilizumab, Pimecrolimus, Placulumab, Plozalizumab, Pomalidomide, Priliximab, purine synthesis inhibitors, pyrimidine synthesis inhibitors, Quilizumab, Reslizumab, Ridaforolimus, Rilonacept, rituximab, Rontalizumab, Rovelizumab, Ruplizumab, Samalizumab, Sarilumab, Secukinumab, Sifalimumab, Siplizumab, Sirolimus, Sirukumab, Sulesomab, sulfasalazine, Tabalumab, Tacrolimus, Talizumab, Telimomab aritox, Temsirolimus, Teneliximab, Teplizumab, Teriflunomide, Tezepelumab, Tildrakizumab, tocilizumab, tofacitinib, Toralizumab, Tralokinumab, Tregalizumab, Tremelimumab, Ulocuplumab, Umirolimus, Urelumab, Ustekinumab, Vapaliximab, Varlilumab, Vatelizumab, Vedolizumab, Vepalimomab, Visilizumab, Vobarilizumab, Zanolimumab, Zolimomab aritox, Zotarolimus, or recombinant human cytokines, such as rh-interferon-gamma.

In some embodiments, a critically ill patient diagnosed with a viral infection is further administered a therapeutically effective dose of a blockade or signaling modification of PD1, PDL1, CTLA4, TIM-3, BTLA, TREM-1, LAG3, VISTA, or any of the human clusters of differentiation, including CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD13, CD14, CD15, CD16, CD16a, CD16b, CD17, CD18, CD19, CD20, CD21, CD22, CD23, CD24, CD25, CD26, CD27, CD28, CD29, CD30, CD31, CD32A, CD32B, CD33, CD34, CD35, CD36, CD37, CD38, CD39, CD40, CD41, CD42, CD42a, CD42b, CD42c, CD42d, CD43, CD44, CD45, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64a, CD65, CD65s, CD66a, CD66b, CD66c, CD66d, CD66e, CD66f, CD68, CD69, CD70, CD71, CD72, CD73, CD74, CD75, CD75s, CD77, CD79A, CD79B, CD80, CD81, CD82, CD83, CD84, CD85A, CD85B, CD85C, CD85D, CD85F, CD85G, CD85H, CD85I, CD85J, CD85K, CD85M, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121a, CD121b, CD122, CD123, CD124, CD125, CD126, CD127, CD129, CD130, CD131, CD132, CD133, CD134, CD135, CD136, CD137, CD138, CD139, CD140A, CD140B, CD141, CD142, CD143, CD144, CDw145, CD146, CD147, CD148, CD150, CD151, CD152, CD153, CD154, CD155, CD156, CD156a, CD156b, CD156c, CD157, CD158, CD158A, CD158B1, CD158B2, CD158C, CD158D, CD158E1, CD158E2, CD158F1, CD158F2, CD158G, CD158H, CD158I, CD158J, CD158K, CD159a, CD159c, CD160, CD161, CD162, CD163, CD164, CD165, CD166, CD167a, CD167b, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD187, CD188, CD189, CD190, CD191, CD192, CD193, CD194, CD195, CD196, CD197, CDw198, CDw199, CD200, CD201, CD202b, CD203c, CD204, CD205, CD206, CD207, CD208, CD209, CD210, CDw210a, CDw210b, CD211, CD212, CD213a1, CD213a2, CD214, CD215, CD216, CD217, CD218a, CD218b, CD219, CD220, CD221, CD222, CD223, CD224, CD225, CD226, CD227, CD228, CD229, CD230, CD231, CD232, CD233, CD234, CD235a, CD235b, CD236, CD237, CD238, CD239, CD240CE, CD240D, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD285, CD286, CD287, CD288, CD289, CD290, CD291, CD292, CDw293, CD294, CD295, CD296, CD297, CD298, CD299, CD300A, CD300C, CD301, CD302, CD303, CD304, CD305, CD306, CD307, CD307a, CD307b, CD307c, CD307d, CD307e, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD344, CD349, CD351, CD352, CD353, CD354, CD355, CD357, CD358, CD360, CD361, CD362, CD363, CD364, CD365, CD366, CD367, CD368, CD369, CD370, or CD371.

In some embodiments, a critically ill patient diagnosed with a viral infection is further administered a therapeutically effective dose of one or more drugs that modify the coagulation cascade or platelet activation, such as those targeting Albumin, Antihemophilic globulin, AHF A, C1-inhibitor, Ca++, CD63, Christmas factor, AHF B, Endothelial cell growth factor, Epidermal growth factor, Factors V, XI, XIII, Fibrin-stabilizing factor, Laki-Lorand factor, fibrinase, Fibrinogen, Fibronectin, GMP 33, Hageman factor, High-molecular-weight kininogen, IgA, IgG, IgM, Interleukin-1B, Multimerin, P-selectin, Plasma thromboplastin antecedent, AHF C, Plasminogen activator inhibitor 1, Platelet factor, Platelet-derived growth factor, Prekallikrein, Proaccelerin, Proconvertin, Protein C, Protein M, Protein S, Prothrombin, Stuart-Prower factor, TF, thromboplastin, Thrombospondin, Tissue factor pathway inhibitor, Transforming growth factor-β, Vascular endothelial growth factor, Vitronectin, von Willebrand factor, α2-Antiplasmin, α2-Macroglobulin, β-Thromboglobulin, or other members of the coagulation or platelet-activation cascades.

In some embodiments, a subject with a respiratory viral infection may be administered agents to control one or more symptoms of the infection, such as analgesics, nonteroidal anti-inflammatory drugs, chemokine receptor blockers, decongestants such as systemic sympathomimetic decongestants, antihistamines, cough suppressants, expectorants, corticosteroids, and others.

In subjects whose viral score indicates an absence or low probability of a viral infection, additional tests can be performed to identify the non-viral cause of the one or more symptoms. For example, in some embodiments, culture tests, blood tests (e.g., full blood count, CRP level, procalcitonin level), Gram staining, PCR, ELISA, or other tests can be performed for bacterial infection using standard methods. In some embodiments, culture tests, microscopic examination, molecular testing (e.g., PCR), antigen testing, Gram staining, or other tests can be performed to detect a fungal infection using standard methods. Medical professionals can also investigate potential other, non-infectious causes (e.g., drugs or toxins, neuromuscular disease, airway disorders, injury, or other conditions, diseases, or disorders) of the observed symptoms.

### VIII. Kits and Systems

### A. Kits

In one aspect, kits are provided for the detection of a respiratory viral infection in a subject, wherein the kits can be used to detect the biomarkers described herein. For example, the kits can be used to detect any one or more of the biomarkers described herein, which are differentially expressed in respiratory samples from subjects with viral infections and from subjects without viral infections. The kit may include one or more agents for the detection of biomarkers, a container for holding a biological sample isolated from a human subject suspected of having a respiratory viral infection; and printed instructions for reacting agents with the biological sample or a portion of the biological sample to detect the presence or amount of at least one biomarker in the biological sample. The agents may be packaged in separate containers. The kit may further comprise one or more control reference samples and reagents for performing a PCR, isothermal amplification, immunoassay, NanoString, or microarray analysis, e.g., reference samples from subjects with or without a viral infection. The kit may also comprise one or more devices or implements for carrying out any of the herein devices, e.g., 96-well plates, microfluidic cartridges, single-well multiplex assays, etc.

In certain embodiments, the kit comprises agents for measuring the levels of at least five or six biomarkers of interest. For example, the kit may include agents, e.g., primers and/or probes, for detecting biomarkers of a panel comprising an IFITM1 polynucleotide, a TLNRD1 polynucleotide, a CDKN1C polynucleotide, an INPP5E polynucleotide, and a TSTD1 polynucleotide, or for detecting any one or more biomarkers listed in Table 2 or Table 3, or one or more pairs of biomarkers listed in Table 4.

In certain embodiments, the kit comprises agents, e.g., primers and/or probes, for measuring the levels of one or more biomarkers (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 90, 95, 100, 200, 300, or all 328 biomarkers) listed in Table 2.

In certain embodiments, the kit comprises agents, e.g., primers and/or probes, for measuring the levels of one or more biomarkers (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, or all 88 biomarkers) listed in Table 3.

In certain embodiments, the kit comprises agents, e.g., primers and/or probes, for measuring the levels of one or more pairs or biomarkers (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, or 500 pairs or biomarkers) listed in Table 4.

In certain embodiments, the kit comprises a microarray or other solid support for analysis of a plurality of biomarker polynucleotides. An exemplary microarray or other support included in the kit comprises an oligonucleotide that hybridizes to an IFITM1 polynucleotide, an oligonucleotide that hybridizes to a TLNRD1 polynucleotide, an oligonucleotide that hybridizes to a CDKN1C polynucleotide, an oligonucleotide that hybridizes to an INPP5E polynucleotide, and an oligonucleotide that hybridizes to a TSTD1 polynucleotide. In some embodiments, the microarray or other support comprises an oligonucleotide for each of the biomarkers detected using the herein-described methods.

The kit can be designed for use with a specific detection system or technique, such as polymerase chain reaction (PCR) (e.g., quantitative PCR (qPCR), droplet digital PCR (ddPCR), reverse transcription PCR (RT-PCR), quantitative RT-PCR (qRT-PCR)), isothermal amplification (e.g., loop-mediated isothermal amplification (LAMP), reverse transcription LAMP (RT-LAMP), quantitative RT-LAMP (qRT-LAMP)), RPA amplification, ligase chain reaction, branched DNA amplification, nucleic acid sequence-based amplification (NASBA), strand displacement assay (SDA), transcription-mediated amplification, rolling circle amplification (RCA), helicase-dependent amplification (HDA), single primer isothermal amplification (SPIA), nicking and extension amplification reaction (NEAR), transcription mediated assay (TMA), CRISPR-Cas detection, or direct hybridization without amplification onto a functionalized surface (e.g., using a graphene biosensor). In particular embodiments, the kit can be designed for use with qRT-PCR or qRT-LAMP. The kit can contain additional materials needed for the specific detection system or technique.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing a viral infection.

### B. Measurement Systems for Detecting and Recording Biomarker Expression

In one aspect, a measurement system is provided. Such systems allow, e.g., the detection of biomarker gene expression in a sample and the recording of the data resulting from the detection. The stored data can then be analyzed as described elsewhere herein to determine the virus infection status of a subject. Such systems can comprise assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

An exemplary measurement system is shown in FIG. 5. The system as shown includes a sample 505, an assay device 510, where an assay 508 can be performed on sample 505. For example, sample 505 can be contacted with reagents of assay 508 to provide a signal of a physical characteristic 515. An example of an assay device can be a flow cell that includes probes and/or primers of an assay or a tube through which a droplet moves (with the droplet including the assay). Physical characteristic 515 (e.g., a fluorescence intensity, a voltage, or a current), from the sample is detected by detector 520. Detector 520 can take a measurement at intervals (e.g., periodic intervals) to obtain data points that make up a data signal. In one embodiment, an analog-to-digital converter converts an analog signal from the detector into digital form at a plurality of times. Assay device 510 and detector 520 can form an assay system, e.g., an amplification and detection system that measures biomarker gene expression according to embodiments described herein. A data signal 525 is sent from detector 520 to logic system 530. As an example, data signal 525 can be used to determine expression levels for selected biomarkers. Data signal 525 can include various measurements made at a same time, e.g., different colors of fluorescent dyes or different electrical signals for different molecules of sample 505, and thus data signal 525 can correspond to multiple signals. Data signal 525, either directly or after online processing by Processor 550, may be stored in a local memory 535, an external memory 540, or a storage device 545. System 500 may also include a treatment device 560, which can provide a treatment to the subject. Treatment device 560 can determine a treatment and/or be used to perform a treatment. Examples of such treatment can include surgery, radiation therapy, chemotherapy, immunotherapy, targeted therapy, hormone therapy, and stem cell transplant. Logic system 530 may be connected to treatment device 560, e.g., to provide results of a method described herein. The treatment device may receive inputs from other devices, such as an imaging device and user inputs (e.g., to control the treatment, such as controls over a robotic system).

### Computer Systems and Diagnostic Systems

Certain aspects of the herein-described methods may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that calculates a viral score based on inputted biomarker expression (and optionally other) data, and determines the viral infection status of a subject.

An exemplary computer system is shown in FIG. 6. Any of the computer systems may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. The subsystems shown in FIG. 6 are interconnected via a system bus 65. Additional subsystems such as a printer 64, keyboard 68, storage device(s) 69, monitor 66 (e.g., a display screen, such as an LED), which is coupled to display adapter 72, and others are shown. Peripherals and input/output (I/O) devices, which couple to I/O controller 61, can be connected to the computer system by any number of means known in the art such as input/output (I/O) port 67 (e.g., USB, FireWire®). For example, I/O port 67 or external interface 71 (e.g. Ethernet, Wi-Fi, etc.) can be used to connect computer system 70 to a wide area network such as the Internet, a mouse input device, or a scanner. The interconnection via system bus 65 allows the central processor 63 to communicate with each subsystem and to control the execution of a plurality of instructions from system memory 62 or the storage device(s) 69 (e.g., a fixed disk, such as a hard drive, or optical disk), as well as the exchange of information between subsystems. The system memory 62 and/or the storage device(s) 69 may embody a computer readable medium. Another subsystem is a data collection device 65, such as a camera, microphone, accelerometer, and the like. Any of the data mentioned herein can be output from one component to another component and can be output to the user. A computer system can include a plurality of the same components or subsystems, e.g., connected together by external interface 71, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

In one aspect, the present disclosure provides a computer implemented method for determining the presence or absence of a respiratory viral infection in a patient. The computer performs steps comprising, e.g.: receiving inputted patient data comprising values for the levels of one or more biomarkers in a biological sample from the patient; analyzing the levels of one or more biomarkers and optionally comparing them to respective reference values, e.g., to a housekeeping reference gene for normalization; calculating a viral score for the patient based on the levels of the biomarkers and comparing the score to one or more threshold values to assign the patient to a viral infection status category; and displaying information regarding the viral infection status or probability of a viral infection in the patient. In certain embodiments, the inputted patient data comprises values for the levels of a plurality of biomarkers in a biological sample from the patient, e.g., biomarkers comprising one or more pairs of biomarkers listed in Table 4. In one embodiment, the inputted patient data comprises values for the levels of IFITM1, TLNRD1, CDKN1C, INPP5E, and TSTD1 polynucleotides.

In a further aspect, a diagnostic system is included for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the respiratory viral status (i.e., infected or uninfected) of the subject. For example, the storage component includes instructions for calculating the viral score for the subject based on biomarker expression levels, as described herein. In addition, the storage component may further comprise instructions for performing multivariate linear discriminant analysis (LDA), receiver operating characteristic (ROC) analysis, principal component analysis (PCA), ensemble data mining methods, cell specific significance analysis of microarrays (csSAM), or multi-dimensional protein identification technology (MUDPIT) analysis. The computer processor is coupled to the storage component and configured to execute the instructions stored in the storage component in order to receive patient data and analyze patient data according to one or more algorithms. The display component displays information regarding the diagnosis of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

### IX. Examples

The following examples are offered to illustrate, but not to limit, the claimed invention.

### A. Example 1. Biomarkers in Nasal Swab Samples from Patients with Respiratory Viral Infections

### 1. Summary

Acute respiratory viral infections are not only a common cause of illness, but also contribute to a substantial amount of mortality in children and adults. Any new diagnostic test needs to be more accurate as well as easy to use. Nasal swabs are commonly gathered to test directly for viral or bacterial pathogens, but this method suffers from colonizer false-positives, and is limited to only those pathogens present in the test. Adding a component to a diagnostic test that measures the host immune response (the body's mRNA) as a way to detect an infection may be a useful adjunct to diagnostic testing. We here explored the idea of reading the host-response from nasal swab samples of suspected individuals using multi-cohort analysis of 6 datasets with infected patients and healthy controls. This analysis allowed us to identify 328 mRNAs that distinguish infected from uninfected samples with high accuracy. For assay utility, we further down-selected 88 mRNAs based on the filtering of their expression level and variation of the selected 328 mRNAs. With the 88 mRNAs, we demonstrated that one can effectively select a subset including a single mRNA marker, a pair of 2-mRNAs, an optimal set of 5 mRNAs, or all 88 mRNAs to achieve the similar level of performance for the purpose of distinguishing viral infected patients from healthy controls based on samples from nasal swab. We envision a new diagnostic test being developed with subsets of these signature mRNAs on an established assay system for clinical use of triaging respiratory viral infections from uninfected individuals.

### 2. Data Sets

Gene Expression Omnibus (GEO) was surveyed for transcriptomic data of respiratory viral infections from nasal swab samples. We identified 6 datasets that fit our search criteria with a total of 383 nasal swab samples collected from patients infected with respiratory virus including HRV, influenza, picornavirus, or RSV. With these 6 datasets (GSE113209, GSE11348, GSE117827, GSE41374, GSE93731, GSE97742), we had a total of 146 uninfected controls and 237 infected samples for our multi-cohort analysis. In some studies, these controls were from a group of healthy individuals. In other studies, these controls were samples taken from the same group of infected subjects after they were discharged. In both cases, we treated them as “controls” and compared them against them the infected group as unmatched samples for multi-cohort analysis. Details about each of the studies are provided in Table 1. We also used a RNASeq dataset (GSE156063) consisting of a total of 234 samples from patients with COVID-19 (n=93), other viral (n=100), or non-viral (n=41) acute respiratory illnesses for biomarker down-selection (see dataset 7 of Table 1).

TABLE 1
Details about the six GEO studies used in the present analysis.

| | | | | #Total | | | #Others | | | Sample |
|---|---|---|---|---|---|---|---|---|---|---|
| Dataset | Accession | Platform | Type | Samples | #Control | #Disease | (ARIs) | Viral Type | Age | Source |
| 1 | GSE113209 | GPL16791 | Expression | 56 | 21 | 32 | | Various | 16 | Nasal |
| | | | profiling by | | | | | including | Children, | mucosal |
| | | | high through- | | | | | RSV and | 15 | scrapings |
| | | | put sequencing | | | | | HRV | Infants | |
| 2 | GSE11348 | GPL570 | Expression | 93 | 16 | 15 | | HRV | Adults | Nasal |
| | | | profiling | | | | | (experimental | | epithelial |
| | | | by array | | | | | rhinovirus | | scrapings |
| | | | | | | | | infection) | | |
| 3 | GSE117827 | GPL23126 | Expression | 50 | 6 | 20 | | RSV, PV | Children | Nasal swabs |
| | | | profiling | | | | | | | |
| | | | by array | | | | | | | |
| 4 | GSE41374 | GPL10558 | Expression | 86 | 10 | 76 | | RSV | Children | Nasal wash |
| | | | profiling | | | | | | | |
| | | | by array | | | | | | | |
| 5 | GSE93731 | GPL570 | Expression | 21 | 11 | 11 | | H1N1 | Adults | Nasal swabs |
| | | | profiling | | | | | | | |
| | | | by array | | | | | | | |
| 6 | GSE97742 | GPL10558 | Expression | 166 | 83 | 83 | | RSV, hRV | Children | Nasopharyngeal |
| | | | profiling | | | | | | | swabs |
| | | | by array | | | | | | | |
| 7 | GSE156063 | GPL24676 | Expression | 234 | 0 | 193 | 41 | SARS-COV-2 | Adults? | NP/OP swab |
| | | | profiling by | | | | | | | |
| | | | high through | | | | | | | |
| | | | put sequencing | | | | | | | |

### 3. Methods

The raw data from each of the 6 studies were downloaded and reprocessed by quantile normalization using RMA. The processed data were then used as input to a multi-cohort analysis using the MetaIntegrator package (v2.1.1). Briefly, effect size was calculated for each mRNA within a study between infected and healthy controls as Hedges' g. The pooled or summary effect size across all datasets was computed using DerSimonian & Laird random-effects model. After summarizing the effect size, p-values across all mRNAs were corrected for multiple testing based on Benjamini-Hochberg false discovery rate (FDR). Fisher's sum of logs method was used for combining p-values across studies. Log-sum of p-values that each mRNA is up- or down-regulated was computed along with corresponding p-values. Again, Benjamini-Hochberg method was performed to correct for multiple testing across all mRNAs. For meta-analysis, we performed leave one-study out (LOO) analysis by removing one dataset at a time. A greedy forward search was used to identity a parsimonious set of genes with the greatest discriminatory power to distinguish samples from infected patients from those from uninfected.

A viral score of a measured sample was calculated as the geometric mean of the normalized, log 2-transformed expression of the over-expressed mRNAs minus that of the under-expressed mRNAs, weighted by the number of mRNAs in over- and under-expressed groups. The scores were scaled for comparison between datasets and used for receiving operating curve (ROC) and area under curve (AUC) as characteristic metrics of the selected biomarker performance.

### 4. Results:

Selection of signature mRNAs: Differential expression was assessed at multiple threshold choices of number of studies, effect size (ES), and false discovery rate (FDR). The number of study cutoff refers to the number of studies in which a selected mRNA is present and measured when performing LOO analysis. At |ES|≥0.6 and FDR≤0.1, a threshold that corresponds 80% power for moderate heterogeneity, we identified 328 differentially expressed mRNAs in 5 out of the 6 studies (and 308 mRNAs in all 6 studies). We decided to use the 328-mRNA list as our biomarker candidate base. Among the 328 mRNAs, 283 are over-expressed and 45 are under-expressed in infected samples in comparison with healthy controls, respectively. The 328 mRNAs are listed in Table 2.

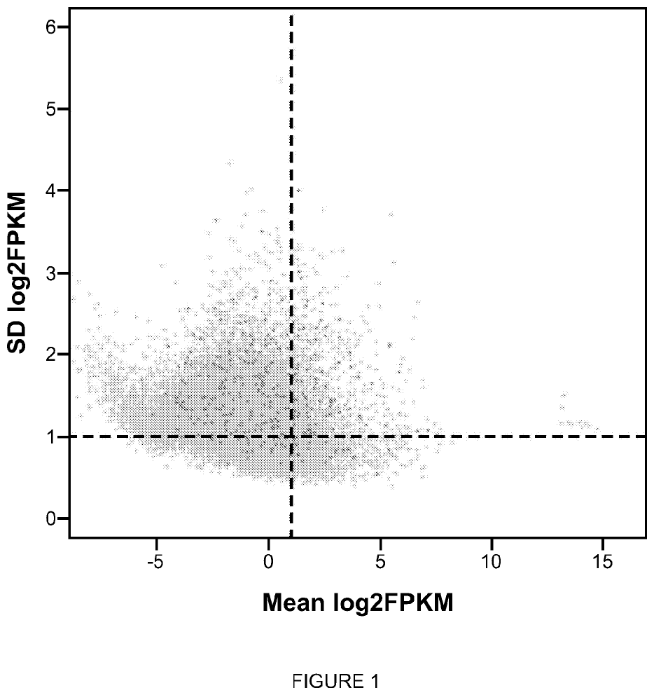

Further filtering of signature mRNAs: These selected 383 mRNAs were further filtered based on their expression level in nasal swab samples from viral-infected patients in a RNASeq dataset (GSE156063). This dataset is reserved for this use because it has no healthy controls. Specifically, we calculated the mean and standard deviation of log 2 FPKM for all the genes across all the 234 samples. From the 328 biomarkers selected above, we chose 88 genes whose mean and standard deviation of log 2FPKM are both greater than 1 (FIG. 1). The 88 mRNAs are flagged in the last column of Table 2, with 80 up-regulated and 8 down-regulated in viral infected samples relative to healthy controls, and are shown in Table 3.

Performance of individual mRNA and two-mRNA combinations: We determined the area under curve (AUC) for receiver operating characteristic (ROC) curve for each of all 12,678 mRNAs with measurements across the 6 studies to understand the background characteristics (FIG. 2A), for each of the 80 selected up-regulated signature mRNAs (FIG. 2B), and for each of the 8 selected down-regulated signature mRNAs across all 6 studies (FIG. 2C). Additionally, we determined the performance of all 3,828 combinations of 2 mRNAs out of the 88 mRNAs in those datasets (FIG. 2D). As a comparator, we also generated AUCs for 10,000 randomly selected 2-mRNA combinations from all 12,678 genes presented in the 6 datasets (FIG. 2E). As expected, the AUCs for single and paired selected signature mRNAs are meaningful as compared the background AUCs. Noticeably, 3,385 pairs of the 2-mRNA combinations out of the 88 mRNAs have AUC≥0.78 (Table 4), accounting for 88.4% of 3,828 total two-mRNA combinations possible from the 88 mRNAs.

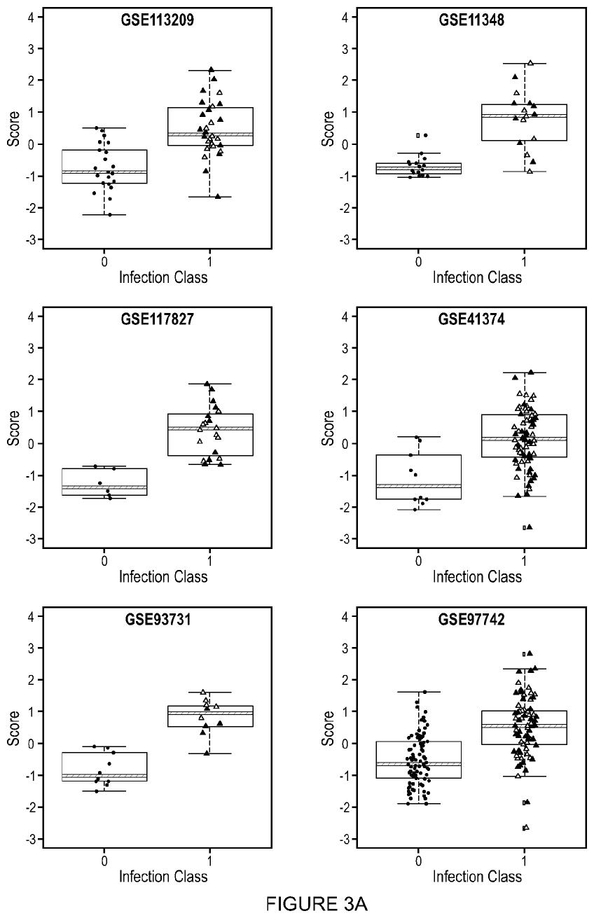

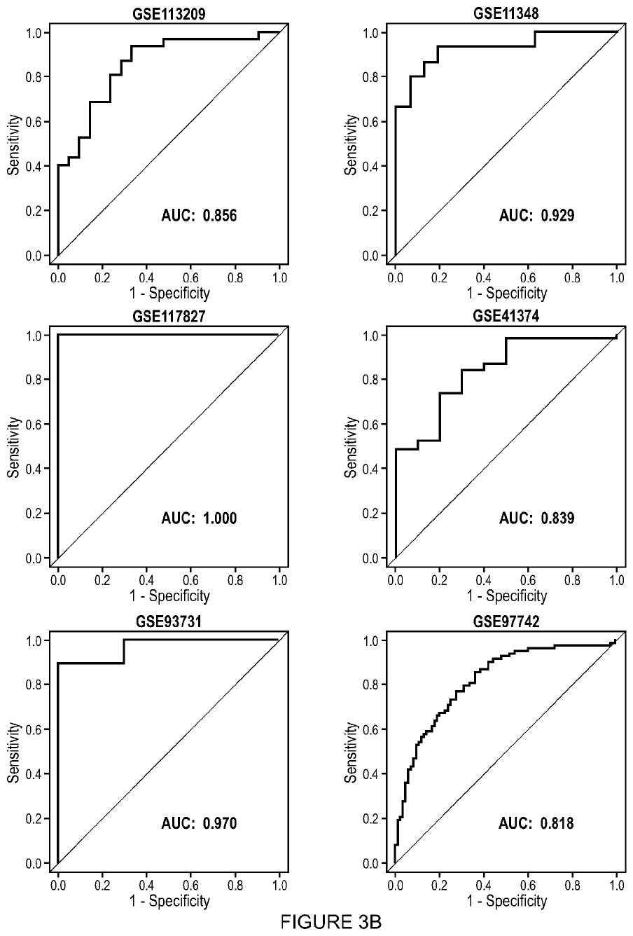

Performance of viral score: The calculated viral score defined as geometric means based on the 88 selected mRNAs were found significantly higher for infected samples as compared to the uninfected samples in all datasets (FIG. 3A). The corresponding AUROC illustrated its high discriminatory power in differentiating infected samples from healthy uninfected controls (FIG. 3B).

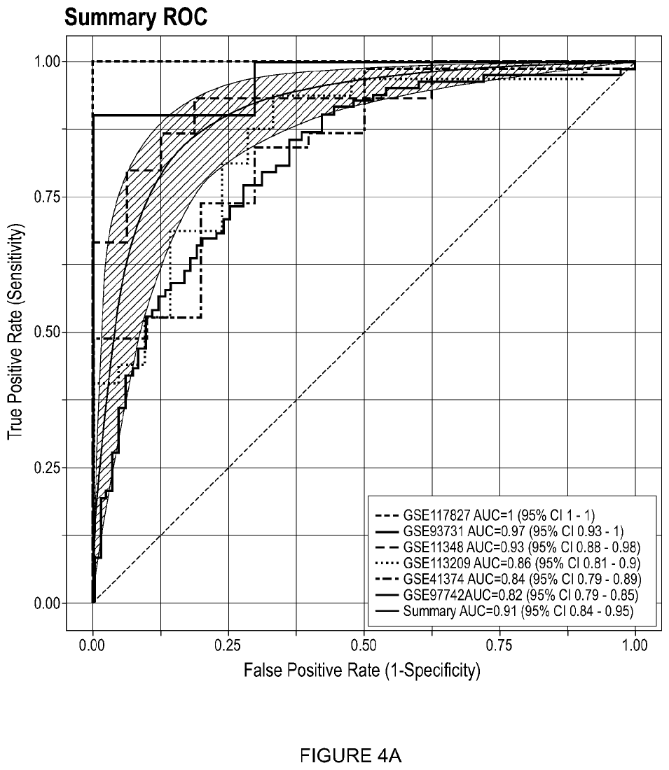

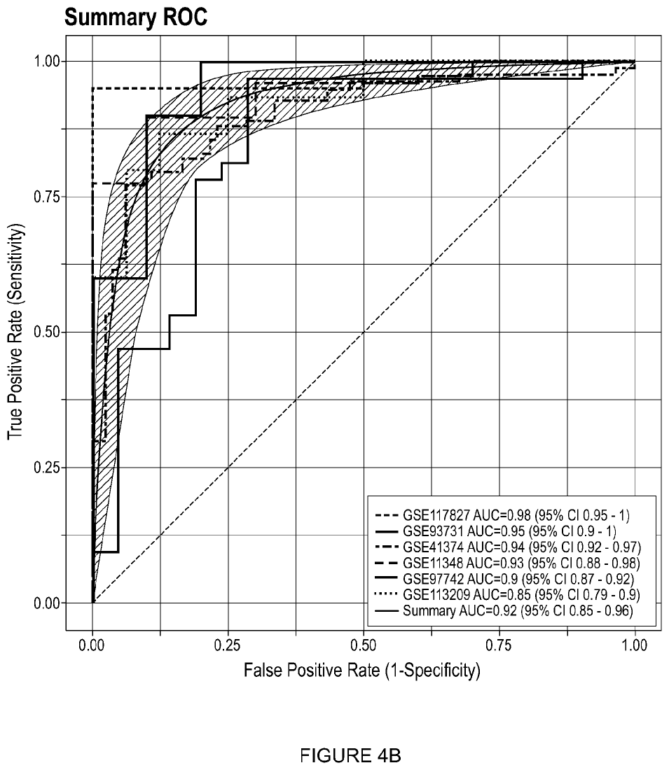

A parsimonious set of signature mRNAs: A greedy forward search algorithm was used to downselect a subset of the signature mRNAs for the optimal discriminatory power. With the 88 signature mRNAs as input, we identified 5 mRNAs (3 up-regulated: IFITM1, TLNRD1, CDKN1C and 2 down-regulated: INPP5E and TSTD1) as a parsimonious set of signature mRNAs. The geometric mean score based on the 5 mRNAs resulted in AUC of 0.92 averaged over the 6 datasets (FIG. 4B) comparable to those for the 88 signature mRNAs (FIG. 4A).

### 5. Discussion

Acute respiratory infections are one of the leading causes for mortality in children and adult. An early accurate diagnosis is needed to quickly identify viral respiratory infections from nasal swab samples. With the 88-mRNA signatures there is a potential to effectively identify viral infection using host response and minimize the unnecessary administration of antibiotics. With the 88 mRNAs, we also demonstrated that one can effectively select a subset of mRNAs either as a single marker of each mRNA marker, a mRNA pair, an optimal set of 5 mRNAs, or all 88 mRNAs together to achieve the similar level of performance for the purpose of distinguishing viral infected patients from healthy controls based on samples from nasal swab.

TABLE 2
The list of 328 mRNAs that distinguish infected vs uninfected samples. These
mRNAs have an absolute effect size > 0.6 and FDR ≤ 0.1 and have been
observed in 5 out of the 6 datasets. Effective size and FDR are given for each
gene. Also listed are mean, standard deviation, and variance of log2 FPKM values.
The last column is the indicator where a gene belongs to the 88 final mRNA list.

| | | | | Mean | SD | | |
|---|---|---|---|---|---|---|---|
| ENTREZ | | Effect | | log2FP | log2FPK | Variance | In 88 |
| ID | SYMBOL | Size | FDR | KM | M | log2FPKM | mRNA list |
| 10578 | GNLY | 1.084 | 6.88E−33 | −1.702 | 2.030 | 4.120 | no |
| 84868 | HAVCR2 | 1.218 | 2.81E−29 | −2.328 | 1.472 | 2.167 | no |
| 64231 | MS4A6A | 0.911 | 2.81E−29 | 1.373 | 1.247 | 1.554 | yes |
| 9332 | CD163 | 1.318 | 2.16E−27 | 0.163 | 1.205 | 1.451 | no |
| 59274 | TLNRD1 | 1.393 | 7.55E−21 | 1.737 | 1.009 | 1.019 | yes |
| 2745 | GLRX | 1.036 | 8.12E−21 | −0.901 | 0.966 | 0.934 | no |
| 10184 | LHFPL2 | 0.993 | 2.29E−20 | −3.985 | 1.242 | 1.544 | no |
| 4481 | MSR1 | 0.694 | 6.27E−20 | −4.014 | 1.081 | 1.169 | no |
| 1200 | TPP1 | 0.926 | 6.31E−19 | 3.071 | 0.830 | 0.688 | no |
| 162073 | ITPRIPL2 | −0.891 | 8.54E−19 | 3.031 | 0.803 | 0.645 | no |
| 170575 | GIMAP1 | 1.041 | 2.42E−17 | −0.867 | 1.890 | 3.572 | no |
| 3689 | ITGB2 | 1.005 | 2.42E−17 | 0.034 | 1.859 | 3.455 | no |
| 128346 | C1orf162 | 0.772 | 2.42E−17 | −0.359 | 2.026 | 4.105 | no |
| 54757 | FAM20A | 1.083 | 3.06E−16 | −1.825 | 1.263 | 1.594 | no |
| 2535 | FZD2 | −0.765 | 3.21E−16 | −2.132 | 1.966 | 3.865 | no |
| 64116 | SLC39A8 | 0.739 | 4.54E−16 | −3.455 | 1.223 | 1.496 | no |
| 151306 | GPBAR1 | 0.666 | 5.76E−16 | −1.075 | 2.248 | 5.052 | no |
| 2022 | ENG | 1.017 | 6.34E−16 | −1.587 | 1.280 | 1.637 | no |
| 23166 | STAB1 | 1.073 | 8.45E−16 | −1.308 | 1.733 | 3.004 | no |
| 10475 | TRIM38 | 1.003 | 8.68E−16 | 1.831 | 0.860 | 0.739 | no |
| 6362 | CCL18 | 0.865 | 2.48E−15 | −3.462 | 1.872 | 3.503 | no |
| 10993 | SDS | 0.839 | 5.54E−15 | −1.518 | 1.450 | 2.101 | no |
| 55340 | GIMAP5 | 0.952 | 8.76E−15 | −1.706 | 2.002 | 4.007 | no |
| 1436 | CSF1R | 0.785 | 8.76E−15 | −1.387 | 1.063 | 1.131 | no |
| 10791 | VAMP5 | 1.023 | 1.48E−14 | −0.259 | 1.617 | 2.615 | no |
| 55803 | ADAP2 | 1.037 | 1.58E−14 | −2.007 | 1.267 | 1.604 | no |
| 55640 | FLVCR2 | 0.825 | 1.71E−14 | −3.637 | 1.325 | 1.755 | no |
| 26157 | GIMAP2 | 0.935 | 1.84E−14 | −0.505 | 1.625 | 2.639 | no |
| 3135 | HLA-G | 0.929 | 2.52E−14 | −2.334 | 1.875 | 3.514 | no |
| 822 | CAPG | 0.959 | 7.78E−14 | 0.171 | 1.175 | 1.381 | no |
| 919 | CD247 | 0.989 | 9.70E−14 | −4.550 | 1.517 | 2.300 | no |
| 3344 | FOXN2 | 0.848 | 1.39E−13 | −1.308 | 0.989 | 0.978 | no |
| 84034 | EMILIN2 | 0.982 | 1.51E−13 | −1.926 | 1.604 | 2.574 | no |
| 155038 | GIMAP8 | 0.852 | 1.52E−13 | −2.765 | 1.863 | 3.469 | no |
| 58475 | MS4A7 | 0.903 | 1.54E−13 | −0.190 | 1.517 | 2.302 | no |
| 2289 | FKBP5 | 0.977 | 1.65E−13 | −1.303 | 1.140 | 1.300 | no |
| 714 | C1QC | −0.742 | 2.59E−13 | 2.142 | 2.007 | 4.030 | yes |
| 941 | CD80 | 0.834 | 3.17E−13 | −3.986 | 1.853 | 3.433 | no |
| 51393 | TRPV2 | 0.895 | 4.03E−13 | −2.425 | 1.885 | 3.553 | no |
| 3101 | HK3 | 0.734 | 4.43E−13 | −2.380 | 2.563 | 6.569 | no |
| 1902 | LPAR1 | 0.816 | 8.99E−13 | −3.854 | 0.990 | 0.979 | no |
| 712 | C1QA | 0.808 | 8.99E−13 | 1.428 | 2.304 | 5.309 | yes |
| 55201 | MAP1S | 0.851 | 1.55E−12 | 0.517 | 0.981 | 0.962 | no |
| 56833 | SLAMF8 | 0.860 | 1.89E−12 | −1.730 | 2.144 | 4.597 | no |
| 8365 | H4C8 | 1.123 | 5.53E−12 | 1.018 | 1.553 | 2.411 | yes |
| 10970 | CKAP4 | 0.911 | 5.67E−12 | −0.820 | 1.062 | 1.128 | no |
| 51131 | PHF11 | 0.914 | 6.66E−12 | 0.180 | 0.811 | 0.658 | no |
| 9049 | AIP | 0.843 | 8.34E−12 | 0.316 | 1.138 | 1.295 | no |
| 9123 | SLC16A3 | 0.787 | 8.60E−12 | 1.702 | 1.476 | 2.177 | yes |
| 6813 | STXBP2 | 0.698 | 9.80E−12 | 2.117 | 1.058 | 1.120 | yes |
| 54676 | GTPBP2 | 1.011 | 1.01E−11 | 0.795 | 0.877 | 0.769 | no |
| 1536 | CYBB | 0.848 | 1.18E−11 | 0.001 | 1.777 | 3.156 | no |
| 55303 | GIMAP4 | 0.727 | 4.30E−11 | 0.905 | 2.244 | 5.035 | no |
| 1845 | DUSP3 | 0.874 | 4.72E−11 | 1.578 | 0.701 | 0.491 | no |
| 2999 | GZMH | 0.946 | 6.95E−11 | −2.093 | 2.430 | 5.907 | no |
| 9711 | RUBCN | −1.044 | 6.98E−11 | −0.495 | 1.143 | 1.305 | no |
| 1028 | CDKN1C | 0.742 | 1.35E−10 | 1.614 | 1.517 | 2.302 | yes |
| 79847 | MFSD13A | 0.860 | 2.02E−10 | −1.781 | 1.801 | 3.245 | no |
| 135112 | NCOA7 | 0.728 | 3.09E−10 | −0.943 | 0.993 | 0.987 | no |
| 3106 | HLA-B | 0.654 | 3.23E−10 | 7.169 | 1.074 | 1.153 | yes |
| 950 | SCARB2 | 0.719 | 4.94E−10 | 0.575 | 0.669 | 0.447 | no |
| 84230 | LRRC8C | 0.940 | 5.52E−10 | −3.771 | 1.358 | 1.845 | no |
| 4818 | NKG7 | 1.190 | 6.16E−10 | 1.437 | 3.011 | 9.066 | yes |
| 6775 | STAT4 | 0.826 | 7.62E−10 | −3.784 | 1.420 | 2.018 | no |
| 4068 | SH2D1A | 0.825 | 7.68E−10 | −5.638 | 1.693 | 2.867 | no |
| 3678 | ITGA5 | 1.589 | 8.34E−10 | −0.755 | 2.231 | 4.978 | no |
| 713 | C1QB | −1.047 | 1.16E−09 | 0.928 | 2.082 | 4.333 | no |
| 55577 | NAGK | 0.653 | 2.43E−09 | 1.300 | 0.982 | 0.965 | no |
| 26579 | MYEOV | 0.990 | 2.59E−09 | −3.369 | 1.321 | 1.745 | no |
| 55106 | SLFN12 | 0.822 | 2.92E−09 | −1.013 | 1.055 | 1.113 | no |
| 313 | AOAH | −0.800 | 3.03E−09 | −3.807 | 1.687 | 2.845 | no |
| 10392 | NOD1 | −0.775 | 3.38E−09 | −2.288 | 1.348 | 1.817 | no |
| 4973 | OLR1 | 1.012 | 3.50E−09 | −0.086 | 1.838 | 3.379 | no |
| 10459 | MAD2L2 | 0.785 | 5.60E−09 | −1.801 | 1.188 | 1.412 | no |
| 6036 | RNASE2 | 0.905 | 7.27E−09 | NA | NA | NA | no |
| 1672 | DEFB1 | 0.778 | 7.95E−09 | −2.035 | 1.828 | 3.343 | no |
| 1240 | CMKLR1 | −0.952 | 8.33E−09 | −3.668 | 1.892 | 3.581 | no |
| 6522 | SLC4A2 | 0.772 | 1.05E−08 | 0.455 | 1.119 | 1.251 | no |
| 22846 | VASH1 | 0.716 | 1.08E−08 | −0.175 | 1.635 | 2.674 | no |
| 140739 | UBE2F | 0.745 | 1.16E−08 | −1.766 | 0.661 | 0.437 | no |
| 64759 | TNS3 | 1.230 | 1.23E−08 | −3.519 | 1.481 | 2.195 | no |
| 81619 | TSPAN14 | 0.763 | 1.89E−08 | 0.198 | 0.845 | 0.715 | no |
| 79690 | GAL3ST4 | 0.784 | 1.93E−08 | −1.456 | 1.671 | 2.794 | no |
| 6507 | SLC1A3 | 0.752 | 2.72E−08 | −3.684 | 1.652 | 2.728 | no |
| 4938 | OAS1 | 0.750 | 3.06E−08 | 1.532 | 1.680 | 2.823 | yes |
| 3071 | NCKAP1L | −0.876 | 4.29E−08 | −0.038 | 1.174 | 1.379 | no |
| 8519 | IFITM1 | −0.689 | 4.62E−08 | 5.482 | 2.110 | 4.453 | yes |
| 57827 | C6orf47 | −0.683 | 6.03E−08 | 2.154 | 1.151 | 1.325 | yes |
| 4245 | MGAT1 | 0.935 | 7.55E−08 | 1.059 | 0.845 | 0.713 | no |
| 2209 | FCGR1A | 1.016 | 7.65E−08 | 0.945 | 1.761 | 3.100 | no |
| 221756 | SERPINB9P1 | 1.128 | 8.05E−08 | NA | NA | NA | no |
| 9168 | TMSB10 | 0.722 | 1.20E−07 | 7.613 | 1.018 | 1.037 | yes |
| 7076 | TIMP1 | 0.950 | 1.20E−07 | 2.325 | 1.434 | 2.058 | yes |
| 3561 | IL2RG | 0.720 | 1.24E−07 | 2.173 | 1.700 | 2.889 | yes |
| 113675 | SDSL | 1.066 | 1.27E−07 | −2.613 | 1.390 | 1.933 | no |
| 56729 | RETN | 0.718 | 1.41E−07 | NA | NA | NA | no |
| 29950 | SERTAD1 | 0.756 | 1.59E−07 | 1.300 | 1.451 | 2.107 | yes |
| 3003 | GZMK | 0.799 | 1.76E−07 | NA | NA | NA | no |
| 51338 | MS4A4A | 0.912 | 1.79E−07 | −5.177 | 1.278 | 1.633 | no |
| 28959 | TMEM176B | 0.698 | 1.79E−07 | −1.660 | 1.926 | 3.708 | no |
| 57493 | HEG1 | 0.784 | 1.97E−07 | −2.498 | 1.462 | 2.138 | no |
| 3002 | GZMB | 0.731 | 1.98E−07 | 0.190 | 2.898 | 8.401 | no |
| 5351 | PLOD1 | 0.735 | 2.97E−07 | −1.903 | 1.140 | 1.300 | no |
| 5973 | RENBP | −0.641 | 3.15E−07 | −1.237 | 1.479 | 2.188 | no |
| 63916 | ELMO2 | 0.788 | 3.44E−07 | −1.101 | 0.819 | 0.670 | no |
| 25903 | OLFML2B | 0.868 | 4.07E−07 | −4.041 | 1.472 | 2.166 | no |
| 286333 | FAM225A | 0.689 | 5.02E−07 | NA | NA | NA | no |
| 1514 | CTSL | 0.698 | 5.22E−07 | 1.783 | 1.374 | 1.887 | yes |
| 921 | CD5 | 0.835 | 1.07E−06 | −2.014 | 1.518 | 2.305 | no |
| 10797 | MTHFD2 | 1.386 | 1.10E−06 | −0.172 | 1.164 | 1.354 | no |
| 3105 | HLA-A | 1.632 | 1.10E−06 | 6.257 | 1.016 | 1.032 | yes |
| 945 | CD33 | 0.807 | 1.11E−06 | −3.900 | 1.530 | 2.342 | no |
| 9935 | MAFB | −0.671 | 1.14E−06 | 3.003 | 1.439 | 2.071 | yes |
| 5551 | PRF1 | −0.838 | 1.17E−06 | 0.093 | 2.742 | 7.516 | no |
| 56935 | SMCO4 | 0.784 | 1.48E−06 | −4.028 | 1.006 | 1.012 | no |
| 914 | CD2 | −0.702 | 1.64E−06 | −0.920 | 2.036 | 4.145 | no |
| 6890 | TAP1 | 1.104 | 1.71E−06 | 3.470 | 1.388 | 1.927 | yes |
| 468 | ATF4 | 1.045 | 1.72E−06 | 4.943 | 0.543 | 0.295 | no |
| 6237 | RRAS | 0.703 | 1.83E−06 | −1.468 | 1.717 | 2.947 | no |
| 54809 | SAMD9 | −0.897 | 1.83E−06 | 2.859 | 1.420 | 2.016 | yes |
| 924 | CD7 | 0.858 | 2.15E−06 | 1.152 | 2.401 | 5.764 | yes |
| 284021 | MILR1 | 0.906 | 2.20E−06 | −2.006 | 1.373 | 1.886 | no |
| 10410 | IFITM3 | 0.813 | 2.58E−06 | 4.054 | 1.726 | 2.978 | yes |
| 9046 | DOK2 | 0.652 | 2.80E−06 | −0.731 | 2.115 | 4.474 | no |
| 4061 | LY6E | 1.111 | 2.99E−06 | 4.419 | 1.137 | 1.294 | yes |
| 168537 | GIMAP7 | 0.879 | 3.06E−06 | −0.990 | 2.399 | 5.754 | no |
| 162461 | TMEM92 | −0.764 | 3.07E−06 | −2.174 | 1.853 | 3.435 | no |
| 126014 | OSCAR | 0.823 | 3.22E−06 | −1.628 | 1.866 | 3.483 | no |
| 3956 | LGALS1 | 0.704 | 3.52E−06 | 1.344 | 1.822 | 3.320 | yes |
| 2537 | IFI6 | 0.737 | 3.81E−06 | 3.249 | 2.423 | 5.873 | yes |
| 79626 | TNFAIP8L2 | 0.744 | 4.30E−06 | −0.655 | 1.891 | 3.576 | no |
| 2210 | FCGR1B | 0.640 | 4.45E−06 | 0.485 | 2.080 | 4.326 | no |
| 83937 | RASSF4 | 0.858 | 4.65E−06 | −1.270 | 1.523 | 2.319 | no |
| 58472 | SQOR | 0.928 | 4.67E−06 | −0.698 | 0.824 | 0.680 | no |
| 65220 | NADK | 1.166 | 4.68E−06 | 1.900 | 1.082 | 1.170 | yes |
| 1890 | TYMP | −1.035 | 4.69E−06 | 5.297 | 1.133 | 1.283 | yes |
| 25819 | NOCT | 0.954 | 4.72E−06 | −1.677 | 1.278 | 1.633 | no |
| 148022 | TICAM1 | 0.960 | 5.04E−06 | −0.146 | 1.383 | 1.912 | no |
| 57168 | ASPHD2 | 0.705 | 5.54E−06 | −2.143 | 1.327 | 1.762 | no |
| 27351 | DESI1 | 0.874 | 6.25E−06 | −0.329 | 1.130 | 1.276 | no |
| 51246 | SHISA5 | 1.024 | 6.29E−06 | 1.350 | 0.782 | 0.612 | no |
| 51251 | NT5C3A | 1.125 | 6.56E−06 | −0.235 | 1.267 | 1.604 | no |
| 2359 | FPR3 | −0.790 | 7.39E−06 | −1.874 | 1.690 | 2.858 | no |
| 126321 | MFSD12 | 1.307 | 7.45E−06 | −0.551 | 1.072 | 1.149 | no |
| 89790 | SIGLEC10 | −0.657 | 7.59E−06 | 1.236 | 1.314 | 1.727 | yes |
| 26270 | FBXO6 | 0.759 | 8.16E−06 | 0.364 | 1.214 | 1.475 | no |
| 147007 | TMEM199 | 1.447 | 8.50E−06 | 1.172 | 1.214 | 1.475 | yes |
| 2040 | STOM | 1.097 | 9.01E−06 | 1.293 | 0.897 | 0.805 | no |
| 2643 | GCH1 | 0.861 | 9.01E−06 | −0.730 | 1.303 | 1.698 | no |
| 2219 | FCN1 | −0.711 | 1.08E−05 | −0.415 | 1.556 | 2.422 | no |
| 8638 | OASL | 0.834 | 1.08E−05 | −0.115 | 2.431 | 5.912 | no |
| 9546 | APBA3 | 0.788 | 1.08E−05 | 0.014 | 1.488 | 2.213 | no |
| 146722 | CD300LF | 0.879 | 1.21E−05 | −2.627 | 1.730 | 2.993 | no |
| 3587 | IL10RA | 0.627 | 1.42E−05 | 0.389 | 2.141 | 4.582 | no |
| 5025 | P2RX4 | 1.057 | 1.54E−05 | 0.270 | 1.098 | 1.206 | no |
| 2896 | GRN | 0.846 | 1.59E−05 | 3.836 | 0.745 | 0.556 | no |
| 2207 | FCER1G | −0.714 | 1.75E−05 | 2.167 | 2.184 | 4.772 | yes |
| 27348 | TOR1B | 0.719 | 1.91E−05 | 1.423 | 1.282 | 1.644 | yes |
| 10581 | IFITM2 | 0.832 | 2.04E−05 | 3.223 | 2.216 | 4.909 | yes |
| 64005 | MYO1G | 1.430 | 2.08E−05 | 0.247 | 1.582 | 2.502 | no |
| 4940 | OAS3 | 0.969 | 2.42E−05 | 1.966 | 1.943 | 3.774 | yes |
| 717 | C2 | 0.883 | 2.42E−05 | −1.617 | 1.040 | 1.082 | no |
| 114769 | CARD16 | 0.649 | 2.51E−05 | −2.261 | 1.977 | 3.910 | no |
| 85363 | TRIM5 | 1.278 | 2.55E−05 | −3.645 | 0.996 | 0.991 | no |
| 11035 | RIPK3 | −0.743 | 2.81E−05 | 1.139 | 1.727 | 2.982 | yes |
| 55603 | TENT5A | 1.296 | 2.82E−05 | −2.468 | 1.016 | 1.032 | no |
| 3134 | HLA-F | 0.768 | 2.95E−05 | 2.003 | 1.341 | 1.797 | yes |
| 51191 | HERC5 | 0.938 | 2.95E−05 | −0.959 | 2.041 | 4.168 | no |
| 730249 | ACOD1 | 0.772 | 3.05E−05 | −3.543 | 2.555 | 6.527 | no |
| 968 | CD68 | 1.179 | 3.22E−05 | 4.193 | 1.216 | 1.478 | yes |
| 3665 | IRF7 | 0.799 | 3.27E−05 | 3.737 | 1.846 | 3.408 | yes |
| 3965 | LGALS9 | 0.916 | 3.76E−05 | 1.876 | 0.924 | 0.853 | no |
| 719 | C3AR1 | 0.875 | 3.78E−05 | −0.120 | 2.177 | 4.740 | no |
| 23643 | LY96 | 0.739 | 3.78E−05 | −3.086 | 1.743 | 3.037 | no |
| 6672 | SP100 | 0.906 | 3.97E−05 | 0.687 | 1.030 | 1.061 | no |
| 9235 | IL32 | 0.808 | 3.97E−05 | −0.361 | 1.765 | 3.114 | no |
| 10384 | BTN3A3 | 1.231 | 4.35E−05 | 0.597 | 1.493 | 2.229 | no |
| 3001 | GZMA | 0.918 | 4.45E−05 | −1.566 | 2.254 | 5.082 | no |
| 79089 | TMUB2 | 1.057 | 4.54E−05 | 1.817 | 1.084 | 1.175 | yes |
| 81030 | ZBP1 | 1.430 | 5.66E−05 | 0.191 | 1.901 | 3.614 | no |
| 661 | POLR3D | −0.936 | 5.98E−05 | 0.107 | 1.423 | 2.024 | no |
| 257019 | FRMD3 | 1.293 | 6.05E−05 | −4.064 | 1.417 | 2.008 | no |
| 7941 | PLA2G7 | 0.814 | 6.16E−05 | −2.791 | 1.475 | 2.174 | no |
| 94240 | EPSTI1 | 1.276 | 6.31E−05 | −1.038 | 1.923 | 3.699 | no |
| 3569 | IL6 | 0.872 | 6.64E−05 | −3.010 | 2.197 | 4.828 | no |
| 11309 | SLCO2B1 | 0.760 | 6.64E−05 | −2.921 | 1.473 | 2.170 | no |
| 85441 | HELZ2 | 0.780 | 7.01E−05 | 3.020 | 1.447 | 2.095 | yes |
| 23586 | DDX58 | 0.971 | 7.04E−05 | −0.173 | 1.731 | 2.997 | no |
| 3434 | IFIT1 | 1.202 | 7.24E−05 | 2.576 | 2.227 | 4.959 | yes |
| 9447 | AIM2 | 1.048 | 7.33E−05 | −3.584 | 1.970 | 3.881 | no |
| 56829 | ZC3HAV1 | −0.670 | 7.52E−05 | 0.874 | 0.984 | 0.969 | no |
| 2014 | EMP3 | 0.741 | 7.65E−05 | −0.400 | 1.573 | 2.473 | no |
| 1316 | KLF6 | 1.222 | 7.74E−05 | 4.095 | 1.002 | 1.003 | yes |
| 3437 | IFIT3 | 1.060 | 8.82E−05 | 2.459 | 2.628 | 6.906 | yes |
| 116071 | BATF2 | 0.727 | 9.35E−05 | −0.076 | 2.111 | 4.457 | no |
| 4924 | NUCB1 | 0.841 | 9.47E−05 | 1.416 | 0.775 | 0.601 | no |
| 3384 | ICAM2 | −0.759 | 9.48E−05 | −1.530 | 1.331 | 1.771 | no |
| 11006 | LILRB4 | 1.185 | 9.48E−05 | −1.087 | 1.633 | 2.667 | no |
| 54739 | XAF1 | 0.840 | 9.60E−05 | 3.240 | 1.293 | 1.671 | yes |
| 9636 | ISG15 | 1.212 | 1.02E−04 | 1.518 | 2.931 | 8.588 | yes |
| 4939 | OAS2 | 0.793 | 1.10E−04 | 2.216 | 1.644 | 2.703 | yes |
| 55365 | TMEM176A | 0.966 | 1.23E−04 | −0.564 | 1.932 | 3.734 | no |
| 55601 | DDX60 | 0.937 | 1.24E−04 | −0.343 | 1.714 | 2.938 | no |
| 710 | SERPING1 | 1.004 | 1.25E−04 | 0.021 | 2.234 | 4.992 | no |
| 8530 | CST7 | 1.150 | 1.28E−04 | −1.258 | 2.386 | 5.692 | no |
| 6355 | CCL8 | 1.246 | 1.48E−04 | −2.318 | 3.638 | 13.235 | no |
| 91624 | NEXN | 1.449 | 1.50E−04 | −2.808 | 1.345 | 1.810 | no |
| 24138 | IFIT5 | 0.839 | 1.57E−04 | 2.623 | 1.614 | 2.604 | yes |
| 969 | CD69 | 0.915 | 1.59E−04 | 0.251 | 2.295 | 5.265 | no |
| 219285 | SAMD9L | 0.728 | 1.76E−04 | 2.453 | 1.794 | 3.219 | yes |
| 3430 | IFI35 | −0.758 | 1.76E−04 | 1.200 | 1.452 | 2.108 | yes |
| 65987 | KCTD14 | 0.874 | 1.76E−04 | −3.125 | 1.203 | 1.447 | no |
| 215 | ABCD1 | 1.207 | 1.83E−04 | −0.887 | 1.435 | 2.058 | no |
| 3433 | IFIT2 | −0.724 | 1.98E−04 | 1.813 | 2.651 | 7.026 | yes |
| 129607 | CMPK2 | 1.352 | 2.01E−04 | 0.554 | 2.062 | 4.250 | no |
| 8651 | SOCS1 | 1.495 | 2.09E−04 | 2.407 | 2.420 | 5.857 | yes |
| 10673 | TNFSF13B | 1.302 | 2.12E−04 | −1.278 | 2.048 | 4.196 | no |
| 91351 | DDX60L | 0.704 | 2.27E−04 | −0.071 | 1.788 | 3.197 | no |
| 23503 | ZFYVE26 | 1.386 | 2.31E−04 | −0.267 | 0.888 | 0.789 | no |
| 56913 | C1GALT1 | 1.943 | 2.41E−04 | −0.845 | 0.906 | 0.821 | no |
| 55332 | DRAM1 | 1.428 | 2.51E−04 | −2.491 | 1.167 | 1.361 | no |
| 3133 | HLA-E | −0.713 | 2.52E−04 | 6.725 | 1.068 | 1.141 | yes |
| 1848 | DUSP6 | 1.434 | 2.59E−04 | 2.633 | 1.252 | 1.567 | yes |
| 64135 | IFIH1 | −0.726 | 2.63E−04 | 0.467 | 1.617 | 2.614 | no |
| 684 | BST2 | 1.004 | 2.64E−04 | 2.652 | 2.331 | 5.432 | yes |
| 4502 | MT2A | 0.977 | 2.70E−04 | 5.909 | 1.792 | 3.211 | yes |
| 8820 | HESX1 | 1.392 | 2.76E−04 | −3.883 | 1.378 | 1.899 | no |
| 282616 | IFNL2 | −0.679 | 2.92E−04 | NA | NA | NA | no |
| 57476 | GRAMD1B | 0.894 | 2.98E−04 | −3.118 | 1.016 | 1.031 | no |
| 60489 | APOBEC3G | −0.897 | 3.00E−04 | 1.210 | 1.143 | 1.306 | yes |
| 3669 | ISG20 | 0.911 | 3.08E−04 | 1.340 | 1.688 | 2.848 | yes |
| 151636 | DTX3L | 1.344 | 3.18E−04 | 3.425 | 0.989 | 0.978 | no |
| 4600 | MX2 | 0.988 | 3.18E−04 | 2.107 | 1.589 | 2.524 | yes |
| 51284 | TLR7 | 0.882 | 3.53E−04 | −2.075 | 1.637 | 2.678 | no |
| 10964 | IFI44L | 0.987 | 3.56E−04 | 2.199 | 2.327 | 5.417 | yes |
| 3601 | IL15RA | −0.746 | 3.61E−04 | −2.822 | 1.319 | 1.739 | no |
| 8743 | TNFSF10 | −0.680 | 3.61E−04 | 2.808 | 1.273 | 1.621 | yes |
| 91543 | RSAD2 | 1.294 | 3.62E−04 | 1.167 | 2.369 | 5.611 | yes |
| 6398 | SECTM1 | 0.934 | 3.68E−04 | 1.357 | 1.875 | 3.514 | yes |
| 1230 | CCR1 | 0.938 | 3.70E−04 | 1.727 | 2.595 | 6.734 | yes |
| 3431 | SP110 | 1.169 | 4.15E−04 | 0.112 | 1.370 | 1.876 | no |
| 79709 | COLGALT1 | 0.931 | 4.35E−04 | −0.282 | 1.084 | 1.175 | no |
| 3903 | LAIR1 | 0.732 | 4.39E−04 | −0.531 | 1.735 | 3.010 | no |
| 10538 | BATF | 0.954 | 4.77E−04 | −2.132 | 1.590 | 2.527 | no |
| 6347 | CCL2 | 1.411 | 4.91E−04 | −0.243 | 3.502 | 12.263 | no |
| 246778 | IL27 | 1.086 | 5.10E−04 | −3.433 | 2.034 | 4.138 | no |
| 838 | CASP5 | 0.800 | 5.33E−04 | −3.304 | 2.241 | 5.022 | no |
| 6773 | STAT2 | 1.146 | 5.38E−04 | 2.928 | 1.095 | 1.199 | yes |
| 5509 | PPP1R3D | 1.231 | 5.94E−04 | 1.938 | 1.036 | 1.074 | yes |
| 3627 | CXCL10 | 0.846 | 6.28E−04 | 1.390 | 4.000 | 16.003 | yes |
| 2633 | GBP1 | 0.762 | 6.31E−04 | 3.223 | 1.901 | 3.614 | yes |
| 57817 | HAMP | 0.715 | 6.31E−04 | −0.629 | 2.176 | 4.735 | no |
| 4599 | MX1 | −1.025 | 6.61E−04 | 2.842 | 1.454 | 2.116 | yes |
| 400759 | GBP1P1 | 1.252 | 6.81E−04 | NA | NA | NA | no |
| 64761 | PARP12 | 0.813 | 7.36E−04 | 0.447 | 1.293 | 1.673 | no |
| 55008 | HERC6 | 1.144 | 7.64E−04 | −0.207 | 1.674 | 2.803 | no |
| 55281 | TMEM140 | 0.826 | 7.83E−04 | 0.731 | 1.490 | 2.221 | no |
| 22797 | TFEC | 0.871 | 9.49E−04 | −4.409 | 2.096 | 4.392 | no |
| 55741 | EDEM2 | 0.851 | 9.51E−04 | −1.835 | 1.072 | 1.149 | no |
| 474344 | GIMAP6 | 1.090 | 9.96E−04 | 0.191 | 2.190 | 4.794 | no |
| 6614 | SIGLEC1 | 0.876 | 1.01E−03 | −1.576 | 2.666 | 7.108 | no |
| 441168 | CALHM6 | 1.555 | 1.04E−03 | 0.659 | 2.660 | 7.075 | no |
| 83666 | PARP9 | 0.852 | 1.04E−03 | 1.798 | 1.239 | 1.534 | yes |
| 10561 | IFI44 | 0.722 | 1.06E−03 | 1.786 | 1.818 | 3.306 | yes |
| 6737 | TRIM21 | −0.768 | 1.09E−03 | 0.941 | 1.348 | 1.817 | no |
| 22809 | ATF5 | 1.034 | 1.13E−03 | 1.730 | 1.463 | 2.140 | yes |
| 10346 | TRIM22 | 0.737 | 1.20E−03 | 1.854 | 1.138 | 1.295 | yes |
| 962 | CD48 | 0.821 | 1.26E−03 | −1.215 | 1.948 | 3.794 | no |
| 11274 | USP18 | −0.941 | 1.27E−03 | −0.598 | 2.033 | 4.132 | no |
| 113730 | KLHDC7B | 0.773 | 1.30E−03 | 1.646 | 2.311 | 5.341 | yes |
| 64108 | RTP4 | 1.511 | 1.47E−03 | 1.346 | 2.227 | 4.957 | yes |
| 10616 | RBCK1 | 0.887 | 1.59E−03 | 1.032 | 0.763 | 0.582 | no |
| 54625 | PARP14 | 0.830 | 2.04E−03 | 2.798 | 1.363 | 1.858 | yes |
| 80830 | APOL6 | 1.028 | 2.05E−03 | 3.621 | 0.838 | 0.702 | no |
| 57823 | SLAMF7 | −0.775 | 2.07E−03 | 0.037 | 2.138 | 4.569 | no |
| 2635 | GBP3 | 1.338 | 2.19E−03 | 2.133 | 1.022 | 1.045 | yes |
| 84875 | PARP10 | −0.869 | 2.24E−03 | 0.286 | 0.974 | 0.949 | no |
| 5610 | EIF2AK2 | 0.963 | 2.26E−03 | 1.673 | 1.047 | 1.096 | yes |
| 51513 | ETV7 | 1.161 | 2.27E−03 | −2.027 | 1.665 | 2.771 | no |
| 118788 | PIK3AP1 | 0.773 | 2.31E−03 | −0.600 | 1.735 | 3.012 | no |
| 834 | CASP1 | 0.882 | 2.37E−03 | 1.758 | 1.390 | 1.932 | yes |
| 23424 | TDRD7 | 0.908 | 2.63E−03 | −1.305 | 0.975 | 0.951 | no |
| 55337 | SHFL | 1.317 | 2.65E−03 | 1.771 | 1.295 | 1.677 | yes |
| 51386 | EIF3L | −0.787 | 2.70E−03 | 0.780 | 0.983 | 0.967 | no |
| 3550 | IK | 0.873 | 2.73E−03 | 3.412 | 0.827 | 0.684 | no |
| 84273 | NOA1 | −0.666 | 2.77E−03 | 0.470 | 1.189 | 1.414 | no |
| 6122 | RPL3 | 1.141 | 2.78E−03 | 5.449 | 0.844 | 0.712 | no |
| 9073 | CLDN8 | 1.157 | 2.79E−03 | −0.534 | 2.698 | 7.279 | no |
| 339512 | CCDC190 | −0.713 | 2.88E−03 | 1.270 | 1.770 | 3.133 | yes |
| 730202 | LOC730202 | 1.203 | 2.97E−03 | NA | NA | NA | no |
| 25874 | MPC2 | −0.719 | 3.00E−03 | 0.456 | 1.122 | 1.258 | no |
| 10969 | EBNA1BP2 | 1.007 | 3.11E−03 | −2.521 | 0.975 | 0.950 | no |
| 114926 | SMIM19 | 0.711 | 3.23E−03 | 0.430 | 1.511 | 2.285 | no |
| 10594 | PRPF8 | 1.097 | 3.23E−03 | 2.622 | 0.685 | 0.470 | no |
| 223 | ALDH9A1 | 0.938 | 3.44E−03 | −0.163 | 0.932 | 0.868 | no |
| 7419 | VDAC3 | 0.963 | 3.44E−03 | 1.181 | 0.959 | 0.920 | no |
| 57223 | PPP4R3B | −0.740 | 3.62E−03 | 1.110 | 0.844 | 0.712 | no |
| 11062 | DUS4L | 0.793 | 3.65E−03 | −1.143 | 1.169 | 1.366 | no |
| 9905 | SGSM2 | 0.768 | 3.78E−03 | 0.628 | 1.027 | 1.055 | no |
| 51805 | COQ3 | 0.949 | 3.95E−03 | −3.279 | 1.156 | 1.337 | no |
| 81706 | PPP1R14C | 0.737 | 3.96E−03 | −2.882 | 1.473 | 2.169 | no |
| 1937 | EEF1G | 0.893 | 4.19E−03 | 3.809 | 0.978 | 0.956 | no |
| 9371 | KIF3B | 0.798 | 4.41E−03 | 1.080 | 0.986 | 0.972 | no |
| 218 | ALDH3A1 | 1.367 | 4.74E−03 | 4.695 | 1.653 | 2.732 | yes |
| 541473 | LOC541473 | 1.004 | 4.90E−03 | NA | NA | NA | no |
| 127262 | TPRG1L | 1.440 | 4.90E−03 | 2.619 | 1.001 | 1.002 | yes |
| 10693 | CCT6B | 0.988 | 4.96E−03 | −2.712 | 1.255 | 1.574 | no |
| 100131187 | TSTD1 | 1.001 | 5.01E−03 | 1.835 | 2.168 | 4.702 | yes |
| 81853 | TMEM14B | 0.795 | 5.77E−03 | −2.100 | 0.922 | 0.851 | no |
| 2067 | ERCC1 | 1.216 | 6.52E−03 | −0.883 | 0.740 | 0.548 | no |
| 5037 | PEBP1 | 0.926 | 6.57E−03 | 2.854 | 0.745 | 0.555 | no |
| 847 | CAT | 0.885 | 7.07E−03 | 0.374 | 0.896 | 0.804 | no |
| 5859 | QARS1 | 0.777 | 7.14E−03 | 2.642 | 0.931 | 0.867 | no |
| 9240 | PNMA1 | 1.200 | 7.18E−03 | 3.696 | 1.342 | 1.801 | yes |
| 10953 | TOMM34 | 1.283 | 7.35E−03 | 0.230 | 1.109 | 1.229 | no |
| 55742 | PARVA | 1.075 | 8.22E−03 | −0.790 | 1.211 | 1.466 | no |
| 9879 | DDX46 | 0.928 | 8.28E−03 | −0.935 | 0.809 | 0.655 | no |
| 25824 | PRDX5 | 0.878 | 8.56E−03 | 4.133 | 1.257 | 1.580 | yes |
| 26061 | HACL1 | −0.800 | 8.74E−03 | −1.875 | 1.178 | 1.387 | no |
| 93099 | DMKN | 0.752 | 9.73E−03 | 0.802 | 1.347 | 1.814 | no |
| 345757 | FAM174A | 1.045 | 1.04E−02 | −1.154 | 1.134 | 1.286 | no |
| 22881 | ANKRD6 | 1.031 | 1.11E−02 | −2.810 | 1.160 | 1.345 | no |
| 10229 | COQ7 | 0.817 | 1.36E−02 | 0.636 | 0.898 | 0.806 | no |
| 2938 | GSTA1 | 0.860 | 1.62E−02 | 3.669 | 2.023 | 4.092 | yes |
| 8863 | PER3 | 1.229 | 1.64E−02 | −0.721 | 1.251 | 1.565 | no |
| 56623 | INPP5E | 1.404 | 1.65E−02 | 1.212 | 1.251 | 1.564 | yes |
| 80263 | TRIM45 | 0.760 | 1.81E−02 | −1.703 | 1.521 | 2.315 | no |
| 3131 | HLF | 0.989 | 2.07E−02 | −1.147 | 1.719 | 2.953 | no |

TABLE 3
List of 88 selected mRNAs.

| | | Effect | | Mean | SD | Variance |
|---|---|---|---|---|---|---|
| ENTREZ ID | SYMBOL | Size | FDR | log2FPKM | log2FPKM | log2FPKM |
| 64231 | MS4A6A | 0.911 | 2.81E−29 | 1.373 | 1.247 | 1.554 |
| 59274 | TLNRD1 | 1.393 | 7.55E−21 | 1.737 | 1.009 | 1.019 |
| 714 | C1QC | −0.742 | 2.59E−13 | 2.142 | 2.007 | 4.030 |
| 712 | C1QA | 0.808 | 8.99E−13 | 1.428 | 2.304 | 5.309 |
| 8365 | H4C8 | 1.123 | 5.53E−12 | 1.018 | 1.553 | 2.411 |
| 9123 | SLC16A3 | 0.787 | 8.60E−12 | 1.702 | 1.476 | 2.177 |
| 6813 | STXBP2 | 0.698 | 9.80E−12 | 2.117 | 1.058 | 1.120 |
| 1028 | CDKN1C | 0.742 | 1.35E−10 | 1.614 | 1.517 | 2.302 |
| 3106 | HLA-B | 0.654 | 3.23E−10 | 7.169 | 1.074 | 1.153 |
| 4818 | NKG7 | 1.190 | 6.16E−10 | 1.437 | 3.011 | 9.066 |
| 4938 | OAS1 | 0.750 | 3.06E−08 | 1.532 | 1.680 | 2.823 |
| 8519 | IFITM1 | −0.689 | 4.62E−08 | 5.482 | 2.110 | 4.453 |
| 57827 | C6orf47 | −0.683 | 6.03E−08 | 2.154 | 1.151 | 1.325 |
| 9168 | TMSB10 | 0.722 | 1.20E−07 | 7.613 | 1.018 | 1.037 |
| 7076 | TIMP1 | 0.950 | 1.20E−07 | 2.325 | 1.434 | 2.058 |
| 3561 | IL2RG | 0.720 | 1.24E−07 | 2.173 | 1.700 | 2.889 |
| 29950 | SERTAD1 | 0.756 | 1.59E−07 | 1.300 | 1.451 | 2.107 |
| 1514 | CTSL | 0.698 | 5.22E−07 | 1.783 | 1.374 | 1.887 |
| 3105 | HLA-A | 1.632 | 1.10E−06 | 6.257 | 1.016 | 1.032 |
| 9935 | MAFB | −0.671 | 1.14E−06 | 3.003 | 1.439 | 2.071 |
| 6890 | TAP1 | 1.104 | 1.71E−06 | 3.470 | 1.388 | 1.927 |
| 54809 | SAMD9 | −0.897 | 1.83E−06 | 2.859 | 1.420 | 2.016 |
| 924 | CD7 | 0.858 | 2.15E−06 | 1.152 | 2.401 | 5.764 |
| 10410 | IFITM3 | 0.813 | 2.58E−06 | 4.054 | 1.726 | 2.978 |
| 4061 | LY6E | 1.111 | 2.99E−06 | 4.419 | 1.137 | 1.294 |
| 3956 | LGALS1 | 0.704 | 3.52E−06 | 1.344 | 1.822 | 3.320 |
| 2537 | IFI6 | 0.737 | 3.81E−06 | 3.249 | 2.423 | 5.873 |
| 65220 | NADK | 1.166 | 4.68E−06 | 1.900 | 1.082 | 1.170 |
| 1890 | TYMP | −1.035 | 4.69E−06 | 5.297 | 1.133 | 1.283 |
| 89790 | SIGLEC10 | −0.657 | 7.59E−06 | 1.236 | 1.314 | 1.727 |
| 147007 | TMEM199 | 1.447 | 8.50E−06 | 1.172 | 1.214 | 1.475 |
| 2207 | FCER1G | −0.714 | 1.75E−05 | 2.167 | 2.184 | 4.772 |
| 27348 | TOR1B | 0.719 | 1.91E−05 | 1.423 | 1.282 | 1.644 |
| 10581 | IFITM2 | 0.832 | 2.04E−05 | 3.223 | 2.216 | 4.909 |
| 4940 | OAS3 | 0.969 | 2.42E−05 | 1.966 | 1.943 | 3.774 |
| 11035 | RIPK3 | −0.743 | 2.81E−05 | 1.139 | 1.727 | 2.982 |
| 3134 | HLA-F | 0.768 | 2.95E−05 | 2.003 | 1.341 | 1.797 |
| 968 | CD68 | 1.179 | 3.22E−05 | 4.193 | 1.216 | 1.478 |
| 3665 | IRF7 | 0.799 | 3.27E−05 | 3.737 | 1.846 | 3.408 |
| 79089 | TMUB2 | 1.057 | 4.54E−05 | 1.817 | 1.084 | 1.175 |
| 85441 | HELZ2 | 0.780 | 7.01E−05 | 3.020 | 1.447 | 2.095 |
| 3434 | IFIT1 | 1.202 | 7.24E−05 | 2.576 | 2.227 | 4.959 |
| 1316 | KLF6 | 1.222 | 7.74E−05 | 4.095 | 1.002 | 1.003 |
| 3437 | IFIT3 | 1.060 | 8.82E−05 | 2.459 | 2.628 | 6.906 |
| 54739 | XAF1 | 0.840 | 9.60E−05 | 3.240 | 1.293 | 1.671 |
| 9636 | ISG15 | 1.212 | 1.02E−04 | 1.518 | 2.931 | 8.588 |
| 4939 | OAS2 | 0.793 | 1.10E−04 | 2.216 | 1.644 | 2.703 |
| 24138 | IFIT5 | 0.839 | 1.57E−04 | 2.623 | 1.614 | 2.604 |
| 219285 | SAMD9L | 0.728 | 1.76E−04 | 2.453 | 1.794 | 3.219 |
| 3430 | IFI35 | −0.758 | 1.76E−04 | 1.200 | 1.452 | 2.108 |
| 3433 | IFIT2 | −0.724 | 1.98E−04 | 1.813 | 2.651 | 7.026 |
| 8651 | SOCS1 | 1.495 | 2.09E−04 | 2.407 | 2.420 | 5.857 |
| 3133 | HLA-E | −0.713 | 2.52E−04 | 6.725 | 1.068 | 1.141 |
| 1848 | DUSP6 | 1.434 | 2.59E−04 | 2.633 | 1.252 | 1.567 |
| 684 | BST2 | 1.004 | 2.64E−04 | 2.652 | 2.331 | 5.432 |
| 4502 | MT2A | 0.977 | 2.70E−04 | 5.909 | 1.792 | 3.211 |
| 60489 | APOBEC3G | −0.897 | 3.00E−04 | 1.210 | 1.143 | 1.306 |
| 3669 | ISG20 | 0.911 | 3.08E−04 | 1.340 | 1.688 | 2.848 |
| 4600 | MX2 | 0.988 | 3.18E−04 | 2.107 | 1.589 | 2.524 |
| 10964 | IF144L | 0.987 | 3.56E−04 | 2.199 | 2.327 | 5.417 |
| 8743 | TNFSF10 | −0.680 | 3.61E−04 | 2.808 | 1.273 | 1.621 |
| 91543 | RSAD2 | 1.294 | 3.62E−04 | 1.167 | 2.369 | 5.611 |
| 6398 | SECTM1 | 0.934 | 3.68E−04 | 1.357 | 1.875 | 3.514 |
| 1230 | CCR1 | 0.938 | 3.70E−04 | 1.727 | 2.595 | 6.734 |
| 6773 | STAT2 | 1.146 | 5.38E−04 | 2.928 | 1.095 | 1.199 |
| 5509 | PPP1R3D | 1.231 | 5.94E−04 | 1.938 | 1.036 | 1.074 |
| 3627 | CXCL10 | 0.846 | 6.28E−04 | 1.390 | 4.000 | 16.003 |
| 2633 | GBP1 | 0.762 | 6.31E−04 | 3.223 | 1.901 | 3.614 |
| 4599 | MX1 | −1.025 | 6.61E−04 | 2.842 | 1.454 | 2.116 |
| 83666 | PARP9 | 0.852 | 1.04E−03 | 1.798 | 1.239 | 1.534 |
| 10561 | IFI44 | 0.722 | 1.06E−03 | 1.786 | 1.818 | 3.306 |
| 22809 | ATF5 | 1.034 | 1.13E−03 | 1.730 | 1.463 | 2.140 |
| 10346 | TRIM22 | 0.737 | 1.20E−03 | 1.854 | 1.138 | 1.295 |
| 113730 | KLHDC7B | 0.773 | 1.30E−03 | 1.646 | 2.311 | 5.341 |
| 64108 | RTP4 | 1.511 | 1.47E−03 | 1.346 | 2.227 | 4.957 |
| 54625 | PARP14 | 0.830 | 2.04E−03 | 2.798 | 1.363 | 1.858 |
| 2635 | GBP3 | 1.338 | 2.19E−03 | 2.133 | 1.022 | 1.045 |
| 5610 | EIF2AK2 | 0.963 | 2.26E−03 | 1.673 | 1.047 | 1.096 |
| 834 | CASP1 | 0.882 | 2.37E−03 | 1.758 | 1.390 | 1.932 |
| 55337 | SHFL | 1.317 | 2.65E−03 | 1.771 | 1.295 | 1.677 |
| 339512 | CCDC190 | −0.713 | 2.88E−03 | 1.270 | 1.770 | 3.133 |
| 218 | ALDH3A1 | 1.367 | 4.74E−03 | 4.695 | 1.653 | 2.732 |
| 127262 | TPRG1L | 1.440 | 4.90E−03 | 2.619 | 1.001 | 1.002 |
| 100131187 | TSTD1 | 1.001 | 5.01E−03 | 1.835 | 2.168 | 4.702 |
| 9240 | PNMA1 | 1.200 | 7.18E−03 | 3.696 | 1.342 | 1.801 |
| 25824 | PRDX5 | 0.878 | 8.56E−03 | 4.133 | 1.257 | 1.580 |
| 2938 | GSTA1 | 0.860 | 1.62E−02 | 3.669 | 2.023 | 4.092 |
| 56623 | INPP5E | 1.404 | 1.65E−02 | 1.212 | 1.251 | 1.564 |

TABLE 4
Performance of 2-gene combinations out of the 88 mRNAs.
We calculated AUC for all 2-mRNA pairs (a total of
3,828 combinations) to evaluate the performance of
our selected 88 mRNAs. Here we list 3,385 gene pairs
that resulted in average AUC ≥0.78 over 6 datasets.

| | | Mean |
|---|---|---|
| Gene1 | Gene2 | AUC |
| ISG15 | PPP1R3D | 0.914 |
| CDKN1C | IFITM1 | 0.912 |
| STXBP2 | ISG15 | 0.906 |
| STXBP2 | IFI6 | 0.905 |
| TLNRD1 | IFITM1 | 0.905 |
| TLNRD1 | ISG15 | 0.905 |
| TMUB2 | ISG15 | 0.905 |
| ISG15 | CCDC190 | 0.904 |
| IFITM1 | LY6E | 0.903 |
| MAFB | ISG15 | 0.903 |
| IFITM1 | TSTD1 | 0.903 |
| NKG7 | ISG15 | 0.903 |
| MS4A6A | IFI6 | 0.902 |
| MS4A6A | ISG15 | 0.902 |
| CDKN1C | IFITM3 | 0.902 |
| CDKN1C | ISG15 | 0.901 |
| IFITM3 | CCDC190 | 0.901 |
| STXBP2 | IFITM1 | 0.901 |
| ISG15 | GSTA1 | 0.901 |
| LY6E | INPP5E | 0.900 |
| C1QA | ISG15 | 0.900 |
| MAFB | LY6E | 0.900 |
| IFI6 | CCDC190 | 0.900 |
| LGALS1 | IF16 | 0.899 |
| IFITM1 | TPRG1L | 0.899 |
| IFITM1 | PRDX5 | 0.898 |
| MS4A6A | IFITM3 | 0.898 |
| ISG15 | INPP5E | 0.898 |
| MS4A6A | IFITM1 | 0.898 |
| ISG15 | TSTD1 | 0.897 |
| IFITM1 | INPP5E | 0.897 |
| IFITM1 | CCDC190 | 0.897 |
| IFI6 | PRDX5 | 0.896 |
| IFITM1 | ISG15 | 0.896 |
| ISG15 | PRDX5 | 0.896 |
| IFITM1 | LGALS1 | 0.896 |
| SLC16A3 | ISG15 | 0.895 |
| IFITM3 | TMUB2 | 0.895 |
| CTSL | ISG15 | 0.895 |
| TMEM199 | ISG15 | 0.895 |
| IFI6 | GSTA1 | 0.895 |
| C6orf47 | ISG15 | 0.894 |
| IFITM1 | MAFB | 0.894 |
| IFI44 | CCDC190 | 0.894 |
| IFITM1 | PNMA1 | 0.894 |
| IFITM3 | TSTD1 | 0.894 |
| IFITM3 | ISG15 | 0.894 |
| MAFB | IFI6 | 0.893 |
| TIMP1 | ISG15 | 0.893 |
| SERTAD1 | ISG15 | 0.893 |
| IFITM1 | TMSB10 | 0.893 |
| LY6E | ISG15 | 0.893 |
| IFITM1 | TIMP1 | 0.892 |
| IFI44L | CCDC190 | 0.892 |
| STXBP2 | IFITM3 | 0.892 |
| CD7 | ISG15 | 0.892 |
| IFITM3 | INPP5E | 0.892 |
| IFITM1 | C6orf47 | 0.892 |
| LGALS1 | ISG15 | 0.892 |
| IFIT1 | CCDC190 | 0.892 |
| H4C8 | ISG15 | 0.891 |
| IFITM3 | LGALS1 | 0.891 |
| HLA-A | ISG15 | 0.891 |
| MAFB | IFITM3 | 0.891 |
| TMSB10 | ISG15 | 0.891 |
| ISG15 | PNMA1 | 0.891 |
| IFITM1 | IFITM3 | 0.890 |
| LY6E | CCDC190 | 0.890 |
| MS4A6A | LY6E | 0.890 |
| IFI6 | INPP5E | 0.890 |
| HLA-B | ISG15 | 0.889 |
| ISG15 | HLA-E | 0.889 |
| MAFB | XAF1 | 0.889 |
| MS4A6A | IFIT1 | 0.889 |
| IFI35 | CCDC190 | 0.889 |
| IFI44L | GSTA1 | 0.889 |
| IFITM1 | TMEM199 | 0.888 |
| IFI6 | TMUB2 | 0.888 |
| LY6E | LGALS1 | 0.888 |
| C1QA | IFITM1 | 0.888 |
| C1QC | ISG15 | 0.888 |
| TIMP1 | LY6E | 0.888 |
| TIMP1 | IFI6 | 0.887 |
| IFITM3 | PRDX5 | 0.887 |
| IFITM1 | IFI6 | 0.887 |
| LY6E | IFITM2 | 0.887 |
| NKG7 | IFITM1 | 0.887 |
| STXBP2 | IFIT1 | 0.887 |
| IFITM1 | CTSL | 0.887 |
| IFIT1 | GSTA1 | 0.887 |
| C1QA | IFITM3 | 0.887 |
| IFI35 | GSTA1 | 0.887 |
| XAF1 | CCDC190 | 0.886 |
| LY6E | GSTA1 | 0.886 |
| FCER1G | ISG15 | 0.886 |
| IFITM1 | HLA-A | 0.886 |
| CDKN1C | IFITM2 | 0.886 |
| MS4A6A | OAS3 | 0.886 |
| SLC16A3 | IFI6 | 0.885 |
| IFITM3 | LY6E | 0.885 |
| TLNRD1 | IFITM3 | 0.885 |
| SLC16A3 | LY6E | 0.885 |
| LGALS1 | IFI35 | 0.885 |
| STXBP2 | OAS3 | 0.885 |
| CD68 | ISG15 | 0.885 |
| IFITM3 | GSTA1 | 0.885 |
| CTSL | IFITM3 | 0.884 |
| IFITM2 | ISG15 | 0.884 |
| CDKN1C | LGALS1 | 0.884 |
| ISG15 | SECTM1 | 0.884 |
| C6orf47 | IFITM3 | 0.884 |
| OAS2 | GSTA1 | 0.884 |
| NADK | ISG15 | 0.884 |
| IFITM3 | PNMA1 | 0.884 |
| TAP1 | ISG15 | 0.884 |
| LY6E | TMUB2 | 0.884 |
| TOR1B | ISG15 | 0.883 |
| TYMP | ISG15 | 0.883 |
| HLA-F | ISG15 | 0.883 |
| ISG15 | DUSP6 | 0.883 |
| IFITM1 | IFI35 | 0.883 |
| TMSB10 | GSTA1 | 0.883 |
| IFITM1 | CD68 | 0.883 |
| IFITM3 | SECTM1 | 0.883 |
| STXBP2 | IFIT2 | 0.883 |
| CDKN1C | FCER1G | 0.883 |
| LGALS1 | OAS3 | 0.883 |
| OAS1 | ISG15 | 0.883 |
| OAS2 | CCDC190 | 0.883 |
| NKG7 | IFIT1 | 0.882 |
| LY6E | TSTD1 | 0.882 |
| IFITM1 | TMUB2 | 0.882 |
| CDKN1C | XAF1 | 0.882 |
| IFITM3 | TMEM199 | 0.882 |
| IFI6 | FCER1G | 0.882 |
| RTP4 | CCDC190 | 0.882 |
| ISG15 | TPRG1L | 0.882 |
| TIMP1 | IFITM3 | 0.882 |
| IFITM3 | IFI6 | 0.882 |
| SERTAD1 | IFITM3 | 0.882 |
| NKG7 | XAF1 | 0.882 |
| IFI6 | PNMA1 | 0.882 |
| SLC16A3 | IFITM3 | 0.881 |
| NKG7 | IFI6 | 0.881 |
| SIGLEC10 | ISG15 | 0.881 |
| IFITM3 | TPRG1L | 0.881 |
| OAS1 | IFITM1 | 0.881 |
| IFI35 | PRDX5 | 0.881 |
| IFI44 | GSTA1 | 0.881 |
| C1QC | IFITM1 | 0.881 |
| OAS3 | CCDC190 | 0.881 |
| RTP4 | GSTA1 | 0.881 |
| CDKN1C | OAS3 | 0.881 |
| SERTAD1 | IFI6 | 0.881 |
| LGALS1 | IFIT1 | 0.880 |
| IFI6 | TSTD1 | 0.880 |
| IFI44L | INPP5E | 0.880 |
| IFITM3 | CD68 | 0.880 |
| MX1 | CCDC190 | 0.880 |
| OAS1 | LGALS1 | 0.880 |
| IFITM1 | XAF1 | 0.879 |
| TIMP1 | IFI44L | 0.879 |
| IFI6 | ISG15 | 0.879 |
| IFIT1 | PRDX5 | 0.879 |
| MS4A6A | NKG7 | 0.879 |
| IFITM1 | SERTAD1 | 0.878 |
| IFI6 | IRF7 | 0.878 |
| NKG7 | IFIT2 | 0.878 |
| MS4A6A | SECTM1 | 0.878 |
| IFITM1 | OAS3 | 0.878 |
| HLA-A | IFITM3 | 0.878 |
| MAFB | IFIT1 | 0.878 |
| RSAD2 | CCDC190 | 0.878 |
| OAS3 | ISG15 | 0.878 |
| IRF7 | ISG15 | 0.877 |
| IFI44L | PNMA1 | 0.877 |
| IFIT1 | INPP5E | 0.877 |
| MS4A6A | IFITM2 | 0.877 |
| MS4A6A | XAF1 | 0.877 |
| LY6E | FCER1G | 0.877 |
| LY6E | IFIT2 | 0.877 |
| LGALS1 | IFIT2 | 0.877 |
| IFITM1 | SECTM1 | 0.877 |
| CTSL | IFI6 | 0.877 |
| RIPK3 | ISG15 | 0.877 |
| OAS3 | INPP5E | 0.877 |
| FCER1G | IFI44L | 0.877 |
| IFI44L | PRDX5 | 0.877 |
| CDKN1C | IFIT2 | 0.876 |
| CTSL | IFIT1 | 0.876 |
| LGALS1 | IFI44L | 0.876 |
| MT2A | CCDC190 | 0.876 |
| MS4A6A | IFI35 | 0.876 |
| LGALS1 | XAF1 | 0.876 |
| SHFL | CCDC190 | 0.876 |
| ISG15 | ALDH3A1 | 0.876 |
| TIMP1 | IFI35 | 0.876 |
| ISG15 | SHFL | 0.876 |
| IRF7 | CCDC190 | 0.876 |
| IFITM3 | IFI35 | 0.876 |
| IFI6 | DUSP6 | 0.876 |
| TLNRD1 | LY6E | 0.875 |
| RSAD2 | GSTA1 | 0.875 |
| CDKN1C | NKG7 | 0.875 |
| IFITM1 | IRF7 | 0.875 |
| TMUB2 | IFIT1 | 0.875 |
| C1QA | IFIT2 | 0.875 |
| STXBP2 | OAS1 | 0.875 |
| STXBP2 | XAF1 | 0.875 |
| NKG7 | IRF7 | 0.875 |
| IFITM1 | IFIT1 | 0.875 |
| IFITM3 | OAS3 | 0.875 |
| IFITM1 | GSTA1 | 0.875 |
| TLNRD1 | IFI6 | 0.875 |
| CDKN1C | IFIT1 | 0.875 |
| OAS1 | IFITM3 | 0.875 |
| LGALS1 | SHFL | 0.875 |
| IFITM1 | IFITM2 | 0.875 |
| ISG15 | IFI35 | 0.874 |
| ISG15 | CASP1 | 0.874 |
| IFIT1 | TSTD1 | 0.874 |
| XAF1 | INPP5E | 0.874 |
| OAS3 | GSTA1 | 0.874 |
| XAF1 | GSTA1 | 0.874 |
| MAFB | IFI44L | 0.874 |
| IFI6 | CD68 | 0.874 |
| XAF1 | ISG15 | 0.874 |
| OAS1 | GSTA1 | 0.874 |
| TLNRD1 | IFIT1 | 0.874 |
| IFITM1 | SHFL | 0.874 |
| TMSB10 | IFITM3 | 0.874 |
| C1QC | IFITM3 | 0.874 |
| STXBP2 | IFI44L | 0.874 |
| LY6E | IRF7 | 0.874 |
| SAMD9L | CCDC190 | 0.874 |
| C1QA | IFIT1 | 0.874 |
| NKG7 | IFITM3 | 0.874 |
| IFITM1 | MT2A | 0.874 |
| MS4A6A | IFI44L | 0.873 |
| CDKN1C | IRF7 | 0.873 |
| TMUB2 | IFI35 | 0.873 |
| IFITM3 | IFITM2 | 0.873 |
| LY6E | XAF1 | 0.873 |
| MS4A6A | TLNRD1 | 0.873 |
| NKG7 | LY6E | 0.873 |
| NKG7 | LGALS1 | 0.873 |
| NKG7 | FCER1G | 0.873 |
| TIMP1 | OAS3 | 0.873 |
| IL2RG | ISG15 | 0.873 |
| CD7 | LGALS1 | 0.873 |
| LY6E | IFIT1 | 0.873 |
| ISG15 | TNFSF10 | 0.873 |
| C1QA | IRF7 | 0.873 |
| TIMP1 | IFIT1 | 0.873 |
| IFITM3 | FCER1G | 0.873 |
| LGALS1 | MX1 | 0.873 |
| IFITM1 | CD7 | 0.873 |
| LGALS1 | RSAD2 | 0.873 |
| IFI6 | SECTM1 | 0.873 |
| ISG15 | MT2A | 0.873 |
| H4C8 | LGALS1 | 0.873 |
| LGALS1 | IRF7 | 0.873 |
| MS4A6A | SHFL | 0.872 |
| FCER1G | IFIT1 | 0.872 |
| ISG15 | TRIM22 | 0.872 |
| IFI35 | INPP5E | 0.872 |
| MS4A6A | OAS1 | 0.872 |
| IFITM1 | FCER1G | 0.872 |
| HLA-A | LY6E | 0.872 |
| IFI6 | IFITM2 | 0.872 |
| NKG7 | MAFB | 0.872 |
| IFI6 | NADK | 0.872 |
| OAS3 | TMUB2 | 0.872 |
| MS4A6A | CD7 | 0.872 |
| IFITM3 | IFIT1 | 0.872 |
| MS4A6A | CDKN1C | 0.872 |
| MAFB | MX1 | 0.872 |
| IRF7 | INPP5E | 0.872 |
| MS4A6A | TMSB10 | 0.872 |
| IFIT1 | PNMA1 | 0.871 |
| CDKN1C | MAFB | 0.871 |
| CDKN1C | IFI6 | 0.871 |
| NKG7 | OAS3 | 0.871 |
| IFITM1 | HELZ2 | 0.871 |
| LY6E | IFI6 | 0.871 |
| ISG15 | IFIT2 | 0.871 |
| LGALS1 | IFIT3 | 0.871 |
| OAS1 | FCER1G | 0.871 |
| HLA-A | OAS3 | 0.871 |
| LY6E | DUSP6 | 0.871 |
| LY6E | PRDX5 | 0.871 |
| MT2A | GSTA1 | 0.871 |
| MS4A6A | IRF7 | 0.871 |
| MAFB | IRF7 | 0.871 |
| IFITM3 | XAF1 | 0.871 |
| SIGLEC10 | IFI44L | 0.871 |
| SAMD9 | ISG15 | 0.871 |
| MS4A6A | MX1 | 0.871 |
| C1QC | IFIT1 | 0.870 |
| STXBP2 | LY6E | 0.870 |
| CDKN1C | IFI44L | 0.870 |
| OAS1 | MAFB | 0.870 |
| KLF6 | ISG15 | 0.870 |
| TIMP1 | IFI44 | 0.870 |
| LGALS1 | EIF2AK2 | 0.870 |
| OAS1 | CCDC190 | 0.870 |
| C1QA | OAS3 | 0.870 |
| SLC16A3 | IFIT1 | 0.870 |
| IFITM1 | RSAD2 | 0.870 |
| HLA-A | MAFB | 0.870 |
| TAP1 | IFI6 | 0.870 |
| OAS1 | IFI6 | 0.870 |
| MAFB | IFIT2 | 0.870 |
| LY6E | OAS3 | 0.870 |
| TMSB10 | CCDC190 | 0.870 |
| HELZ2 | ISG15 | 0.869 |
| SHFL | INPP5E | 0.869 |
| MX1 | GSTA1 | 0.869 |
| STXBP2 | MX1 | 0.869 |
| TAP1 | LGALS1 | 0.869 |
| IFI6 | TPRG1L | 0.869 |
| STXBP2 | IFIT3 | 0.869 |
| IFITM1 | IFIT2 | 0.869 |
| TMEM199 | IFIT1 | 0.869 |
| ISG15 | RSAD2 | 0.869 |
| SLC16A3 | IFITM1 | 0.869 |
| IFI6 | IFIT2 | 0.869 |
| IFITM2 | IFI44L | 0.869 |
| LGALS1 | SAMD9L | 0.869 |
| ISG15 | EIF2AK2 | 0.869 |
| MS4A6A | IFIT2 | 0.868 |
| MAFB | OAS3 | 0.868 |
| NKG7 | IFITM2 | 0.868 |
| CD7 | IFIT1 | 0.868 |
| IFITM3 | HELZ2 | 0.868 |
| IFIT1 | ISG15 | 0.868 |
| SHFL | GSTA1 | 0.868 |
| TLNRD1 | LGALS1 | 0.868 |
| SLC16A3 | OAS3 | 0.868 |
| IRF7 | GSTA1 | 0.868 |
| TMSB10 | TMUB2 | 0.868 |
| TIMP1 | RSAD2 | 0.868 |
| IFITM3 | SHFL | 0.868 |
| STXBP2 | ISG20 | 0.868 |
| IFITM3 | IRF7 | 0.868 |
| FCER1G | IFI44 | 0.868 |
| ISG15 | KLHDC7B | 0.868 |
| OAS1 | TSTD1 | 0.868 |
| C1QA | LY6E | 0.868 |
| MAFB | IFITM2 | 0.868 |
| LGALS1 | STAT2 | 0.868 |
| LY6E | PNMA1 | 0.868 |
| H4C8 | IFITM1 | 0.867 |
| IFI6 | HLA-E | 0.867 |
| TMUB2 | MX1 | 0.867 |
| RSAD2 | PRDX5 | 0.867 |
| OAS1 | LY6E | 0.867 |
| OAS1 | TIMP1 | 0.867 |
| MS4A6A | MAFB | 0.867 |
| NKG7 | IFI35 | 0.867 |
| IFITM1 | KLHDC7B | 0.867 |
| TIMP1 | XAF1 | 0.867 |
| LY6E | TYMP | 0.867 |
| IFI44L | TSTD1 | 0.867 |
| IFITM1 | IFI44L | 0.867 |
| LGALS1 | TNFSF10 | 0.867 |
| MS4A6A | RSAD2 | 0.867 |
| HLA-B | IFITM1 | 0.866 |
| LGALS1 | TOR1B | 0.866 |
| FCER1G | OAS3 | 0.866 |
| TLNRD1 | XAF1 | 0.866 |
| C6orf47 | IFIT1 | 0.866 |
| LY6E | SECTM1 | 0.866 |
| IFI6 | CASP1 | 0.866 |
| CTSL | GSTA1 | 0.866 |
| IFI44 | INPP5E | 0.866 |
| NKG7 | IFIT3 | 0.866 |
| OAS1 | TMUB2 | 0.866 |
| TMSB10 | LY6E | 0.866 |
| TIMP1 | TAP1 | 0.866 |
| CTSL | IFIT2 | 0.866 |
| OAS3 | TSTD1 | 0.866 |
| MAFB | GSTA1 | 0.866 |
| NKG7 | INPP5E | 0.866 |
| C1QA | IFI6 | 0.866 |
| SERTAD1 | LY6E | 0.866 |
| LY6E | SHFL | 0.866 |
| ISG15 | PARP9 | 0.866 |
| LY6E | NADK | 0.866 |
| IFITM2 | IFIT1 | 0.866 |
| IRF7 | TSTD1 | 0.866 |
| TLNRD1 | IFITM2 | 0.865 |
| IFIT3 | ISG15 | 0.865 |
| ISG15 | IFI44L | 0.865 |
| TMSB10 | IFIT1 | 0.865 |
| HLA-A | IFIT1 | 0.865 |
| TIMP1 | EIF2AK2 | 0.865 |
| HLA-A | XAF1 | 0.865 |
| IFITM3 | RIPK3 | 0.865 |
| LGALS1 | IFIT5 | 0.865 |
| IFI44 | PRDX5 | 0.865 |
| MT2A | INPP5E | 0.865 |
| MS4A6A | HELZ2 | 0.865 |
| TLNRD1 | FCER1G | 0.865 |
| TIMP1 | MX1 | 0.865 |
| TAP1 | LY6E | 0.865 |
| FCER1G | IFI35 | 0.865 |
| MS4A6A | STAT2 | 0.865 |
| C1QA | XAF1 | 0.865 |
| OAS1 | IFIT1 | 0.865 |
| TMSB10 | IFI6 | 0.865 |
| MAFB | SHFL | 0.865 |
| LGALS1 | HELZ2 | 0.865 |
| LGALS1 | MT2A | 0.865 |
| LGALS1 | SECTM1 | 0.865 |
| IFI6 | CCR1 | 0.865 |
| XAF1 | TSTD1 | 0.865 |
| RTP4 | PRDX5 | 0.865 |
| ISG15 | IFIT5 | 0.865 |
| C1QC | IFI6 | 0.864 |
| STXBP2 | IRF7 | 0.864 |
| IFITM2 | IFI35 | 0.864 |
| IFITM1 | MX1 | 0.864 |
| CD7 | IFITM3 | 0.864 |
| RSAD2 | PNMA1 | 0.864 |
| MS4A6A | MT2A | 0.864 |
| MS4A6A | IFI44 | 0.864 |
| SLC16A3 | NKG7 | 0.864 |
| LY6E | CASP1 | 0.864 |
| ISG15 | STAT2 | 0.864 |
| IFI35 | TPRG1L | 0.864 |
| TLNRD1 | IRF7 | 0.864 |
| C6orf47 | IFI6 | 0.864 |
| TMSB10 | XAF1 | 0.864 |
| MAFB | IFIT3 | 0.864 |
| TMUB2 | XAF1 | 0.864 |
| ISG15 | ISG20 | 0.864 |
| ISG15 | SAMD9L | 0.864 |
| IFI35 | PNMA1 | 0.864 |
| C1QA | OAS1 | 0.864 |
| IFITM1 | TAP1 | 0.864 |
| TMSB10 | PRDX5 | 0.864 |
| TMSB10 | LGALS1 | 0.864 |
| CTSL | IFITM2 | 0.864 |
| TMSB10 | MAFB | 0.864 |
| OAS1 | IFITM2 | 0.863 |
| STXBP2 | RSAD2 | 0.863 |
| CDKN1C | TAP1 | 0.863 |
| NKG7 | RSAD2 | 0.863 |
| TAP1 | IFITM3 | 0.863 |
| IFITM3 | IFIT2 | 0.863 |
| IFI6 | PPP1R3D | 0.863 |
| IFI35 | TSTD1 | 0.863 |
| IFITM1 | OAS2 | 0.863 |
| HLA-A | IFI6 | 0.863 |
| LGALS1 | OAS2 | 0.863 |
| ISG15 | ATF5 | 0.863 |
| STXBP2 | IFI35 | 0.863 |
| NKG7 | SHFL | 0.863 |
| LY6E | TPRG1L | 0.863 |
| IFI44 | TSTD1 | 0.863 |
| LGALS1 | IFI44 | 0.863 |
| IFIT1 | SECTM1 | 0.863 |
| HLA-B | IFI6 | 0.863 |
| MAFB | RSAD2 | 0.863 |
| RIPK3 | IFIT1 | 0.863 |
| CD68 | IFIT1 | 0.863 |
| ISG15 | GBP3 | 0.863 |
| NKG7 | PNMA1 | 0.863 |
| HLA-B | IFITM3 | 0.863 |
| NKG7 | HLA-A | 0.863 |
| MAFB | LGALS1 | 0.863 |
| MAFB | MT2A | 0.863 |
| FCER1G | XAF1 | 0.863 |
| TMSB10 | TSTD1 | 0.863 |
| FCER1G | MT2A | 0.863 |
| C1QC | MAFB | 0.862 |
| TMSB10 | IFITM2 | 0.862 |
| OAS2 | PRDX5 | 0.862 |
| IFITM1 | TNFSF10 | 0.862 |
| C1QC | LY6E | 0.862 |
| IFI6 | TMEM199 | 0.862 |
| MS4A6A | IFIT3 | 0.862 |
| C1QC | IFIT2 | 0.862 |
| SLC16A3 | IFI44L | 0.862 |
| NKG7 | OAS1 | 0.862 |
| TIMP1 | IRF7 | 0.862 |
| MAFB | CD7 | 0.862 |
| IFI6 | SIGLEC10 | 0.862 |
| IFITM2 | OAS3 | 0.862 |
| ISG15 | SOCS1 | 0.862 |
| ISG15 | CCR1 | 0.862 |
| OAS1 | PRDX5 | 0.862 |
| H4C8 | IFI6 | 0.862 |
| IFITM1 | STAT2 | 0.862 |
| SERTAD1 | IFIT1 | 0.862 |
| MAFB | IFI35 | 0.862 |
| IFIT2 | CCDC190 | 0.862 |
| TLNRD1 | NKG7 | 0.862 |
| OAS1 | HLA-A | 0.862 |
| MAFB | HLA-F | 0.862 |
| MAFB | BST2 | 0.862 |
| LGALS1 | IFITM2 | 0.862 |
| FCER1G | OAS2 | 0.862 |
| XAF1 | PNMA1 | 0.862 |
| CDKN1C | CCR1 | 0.862 |
| BST2 | CCDC190 | 0.862 |
| IFIT3 | CCDC190 | 0.862 |
| TNFSF10 | CCDC190 | 0.862 |
| H4C8 | MAFB | 0.862 |
| OAS1 | XAF1 | 0.862 |
| IFITM1 | SAMD9 | 0.862 |
| IFITM1 | TYMP | 0.862 |
| SAMD9L | PNMA1 | 0.862 |
| NKG7 | IFI44L | 0.862 |
| IFITM3 | RSAD2 | 0.862 |
| STAT2 | CCDC190 | 0.862 |
| OAS2 | INPP5E | 0.862 |
| MS4A6A | HLA-A | 0.861 |
| C1QA | MAFB | 0.861 |
| H4C8 | IFIT1 | 0.861 |
| STXBP2 | BST2 | 0.861 |
| NKG7 | CD68 | 0.861 |
| OAS3 | XAF1 | 0.861 |
| IFIT2 | MT2A | 0.861 |
| MS4A6A | OAS2 | 0.861 |
| C1QC | OAS3 | 0.861 |
| IFITM3 | IFI44L | 0.861 |
| MS4A6A | TSTD1 | 0.861 |
| C1QA | IFI44L | 0.861 |
| CDKN1C | IFIT3 | 0.861 |
| NKG7 | TIMP1 | 0.861 |
| IFITM1 | HLA-F | 0.861 |
| CTSL | IRF7 | 0.861 |
| IFITM2 | XAF1 | 0.861 |
| IFITM2 | IFI44 | 0.861 |
| IRF7 | IFI44L | 0.861 |
| NKG7 | CCDC190 | 0.861 |
| IFIT3 | GSTA1 | 0.861 |
| H4C8 | IFITM3 | 0.861 |
| OAS1 | IRF7 | 0.861 |
| CTSL | XAF1 | 0.861 |
| SAMD9 | LGALS1 | 0.861 |
| LY6E | CD68 | 0.861 |
| ISG15 | MX1 | 0.861 |
| RSAD2 | TSTD1 | 0.861 |
| SAMD9L | GSTA1 | 0.861 |
| C1QC | XAF1 | 0.861 |
| SLC16A3 | XAF1 | 0.861 |
| STXBP2 | OAS2 | 0.861 |
| IFI6 | IFI35 | 0.861 |
| ISG15 | IFI44 | 0.861 |
| IFIT2 | GSTA1 | 0.861 |
| TIMP1 | IFIT2 | 0.861 |
| CD7 | IFI6 | 0.861 |
| IFITM3 | TYMP | 0.861 |
| LGALS1 | BST2 | 0.861 |
| IFIT1 | IFIT2 | 0.861 |
| IFIT2 | IFI44L | 0.861 |
| IFIT5 | CCDC190 | 0.861 |
| IFI44 | PNMA1 | 0.861 |
| RSAD2 | INPP5E | 0.861 |
| SLC16A3 | IFI35 | 0.860 |
| SAMD9 | IFI6 | 0.860 |
| CD7 | XAF1 | 0.860 |
| IFI6 | RSAD2 | 0.860 |
| XAF1 | SECTM1 | 0.860 |
| CDKN1C | RSAD2 | 0.860 |
| IFITM3 | HLA-F | 0.860 |
| MX1 | INPP5E | 0.860 |
| TIMP1 | IFIT5 | 0.860 |
| IFI6 | OAS3 | 0.860 |
| FCER1G | RTP4 | 0.860 |
| MX1 | PRDX5 | 0.860 |
| MS4A6A | KLHDC7B | 0.860 |
| HLA-A | IRF7 | 0.860 |
| MS4A6A | BST2 | 0.860 |
| TIMP1 | OAS2 | 0.860 |
| TIMP1 | SAMD9L | 0.860 |
| IFITM3 | TNFSF10 | 0.860 |
| IFITM1 | ATF5 | 0.860 |
| IRF7 | XAF1 | 0.860 |
| MS4A6A | GSTA1 | 0.860 |
| SLC16A3 | MT2A | 0.859 |
| TMSB10 | OAS3 | 0.859 |
| HLA-A | IFI35 | 0.859 |
| CD68 | IFIT2 | 0.859 |
| MS4A6A | EIF2AK2 | 0.859 |
| IFITM3 | SAMD9L | 0.859 |
| IFITM3 | MT2A | 0.859 |
| LGALS1 | TRIM22 | 0.859 |
| TYMP | IFIT1 | 0.859 |
| TMEM199 | IFIT2 | 0.859 |
| IFIT1 | MT2A | 0.859 |
| OAS1 | IFIT2 | 0.859 |
| IFI6 | HELZ2 | 0.859 |
| NADK | IFIT1 | 0.859 |
| TMUB2 | RSAD2 | 0.859 |
| HLA-B | IFIT1 | 0.859 |
| NKG7 | IFIT5 | 0.859 |
| IFITM1 | IFIT3 | 0.859 |
| LY6E | ISG20 | 0.859 |
| IFI44L | SECTM1 | 0.859 |
| CDKN1C | OAS1 | 0.859 |
| MAFB | IFI44 | 0.859 |
| LY6E | TOR1B | 0.859 |
| FCER1G | RSAD2 | 0.859 |
| OAS3 | CD68 | 0.859 |
| MT2A | PRDX5 | 0.859 |
| C1QA | LGALS1 | 0.859 |
| CDKN1C | SAMD9L | 0.859 |
| NKG7 | MT2A | 0.859 |
| LY6E | IFI35 | 0.859 |
| HLA-F | IFIT1 | 0.859 |
| OAS1 | INPP5E | 0.859 |
| C1QA | RSAD2 | 0.858 |
| LGALS1 | HLA-F | 0.858 |
| IFI6 | RIPK3 | 0.858 |
| NKG7 | EIF2AK2 | 0.858 |
| TIMP1 | MT2A | 0.858 |
| H4C8 | LY6E | 0.858 |
| MS4A6A | SOCS1 | 0.858 |
| C6orf47 | IFITM2 | 0.858 |
| CD7 | LY6E | 0.858 |
| IRF7 | IFIT1 | 0.858 |
| MS4A6A | CCDC190 | 0.858 |
| IFITM1 | ALDH3A1 | 0.858 |
| IFIT5 | PNMA1 | 0.858 |
| C1QA | IFIT3 | 0.858 |
| TIMP1 | TNFSF10 | 0.858 |
| CTSL | IFIT3 | 0.858 |
| MAFB | TAP1 | 0.858 |
| IFITM3 | NADK | 0.858 |
| TMEM199 | IRF7 | 0.858 |
| OAS3 | IFIT1 | 0.858 |
| IFIT1 | IFI35 | 0.858 |
| MS4A6A | TAP1 | 0.858 |
| C1QC | IRF7 | 0.858 |
| C1QA | SAMD9 | 0.858 |
| NKG7 | MX2 | 0.858 |
| IFI6 | HLA-F | 0.858 |
| ISG15 | CXCL10 | 0.858 |
| OAS2 | TSTD1 | 0.858 |
| C1QC | RSAD2 | 0.858 |
| SLC16A3 | TMSB10 | 0.858 |
| IFITM3 | IFIT5 | 0.858 |
| IFITM3 | ALDH3A1 | 0.858 |
| TNFSF10 | GSTA1 | 0.858 |
| IL2RG | IFI6 | 0.858 |
| MAFB | EIF2AK2 | 0.858 |
| FCER1G | SHFL | 0.858 |
| MT2A | PNMA1 | 0.858 |
| SLC16A3 | OAS1 | 0.857 |
| CDKN1C | OAS2 | 0.857 |
| TIMP1 | IFIT3 | 0.857 |
| CTSL | OAS3 | 0.857 |
| LY6E | TMEM199 | 0.857 |
| NADK | OAS3 | 0.857 |
| EIF2AK2 | CCDC190 | 0.857 |
| XAF1 | PRDX5 | 0.857 |
| IFITM2 | SHFL | 0.857 |
| OAS3 | IRF7 | 0.857 |
| IFIT1 | XAF1 | 0.857 |
| ISG15 | BST2 | 0.857 |
| IFIT1 | TPRG1L | 0.857 |
| MS4A6A | FCER1G | 0.857 |
| TMSB10 | TIMP1 | 0.857 |
| CD7 | IFIT2 | 0.857 |
| FI6 | IFIT3 | 0.857 |
| IFITM2 | IRF7 | 0.857 |
| IFI6 | ALDH3A1 | 0.857 |
| MS4A6A | TYMP | 0.857 |
| HLA-A | RSAD2 | 0.857 |
| IFITM3 | IFIT3 | 0.857 |
| IFITM3 | MX1 | 0.857 |
| FCER1G | MX1 | 0.857 |
| HELZ2 | IFIT1 | 0.857 |
| SLC16A3 | MX1 | 0.857 |
| TMSB10 | IFIT2 | 0.857 |
| TAP1 | IFIT1 | 0.857 |
| IFIT1 | HLA-E | 0.857 |
| LGALS1 | TYMP | 0.857 |
| IFI6 | ISG20 | 0.857 |
| TNFSF10 | PRDX5 | 0.857 |
| LGALS1 | GSTA1 | 0.857 |
| MS4A6A | INPP5E | 0.857 |
| MAFB | SAMD9 | 0.856 |
| LGALS1 | PPP1R3D | 0.856 |
| OAS2 | PNMA1 | 0.856 |
| OAS3 | PRDX5 | 0.856 |
| MS4A6A | IFIT5 | 0.856 |
| IFITM1 | RIPK3 | 0.856 |
| LY6E | IFIT3 | 0.856 |
| IFI6 | IFIT1 | 0.856 |
| MS4A6A | ISG20 | 0.856 |
| IFITM1 | SAMD9L | 0.856 |
| TIMP1 | STAT2 | 0.856 |
| SAMD9 | IFITM3 | 0.856 |
| STXBP2 | NKG7 | 0.856 |
| NKG7 | MX1 | 0.856 |
| IFIT1 | RSAD2 | 0.856 |
| NKG7 | HELZ2 | 0.856 |
| C6orf47 | IRF7 | 0.856 |
| IFI6 | SHFL | 0.856 |
| NADK | IFI35 | 0.856 |
| ISG15 | MX2 | 0.856 |
| NKG7 | SAMD9 | 0.856 |
| CD7 | OAS3 | 0.856 |
| ISG15 | PARP14 | 0.856 |
| BST2 | GSTA1 | 0.856 |
| SLC16A3 | IRF7 | 0.856 |
| TLNRD1 | MAFB | 0.856 |
| TLNRD1 | IFIT2 | 0.856 |
| MAFB | SECTM1 | 0.855 |
| STXBP2 | SAMD9L | 0.855 |
| OAS1 | NADK | 0.855 |
| IFI6 | XAF1 | 0.855 |
| IRF7 | TMUB2 | 0.855 |
| IFIT1 | SHFL | 0.855 |
| NKG7 | SECTM1 | 0.855 |
| HLA-A | LGALS1 | 0.855 |
| IFITM3 | STAT2 | 0.855 |
| LY6E | HELZ2 | 0.855 |
| C1QA | IFITM2 | 0.855 |
| STXBP2 | IFITM2 | 0.855 |
| IL2RG | IFI44L | 0.855 |
| SERTAD1 | IFI44L | 0.855 |
| XAF1 | IFIT2 | 0.855 |
| HLA-E | IFI44L | 0.855 |
| IFIT2 | PRDX5 | 0.855 |
| MS4A6A | TMEM199 | 0.855 |
| HLA-A | IFIT2 | 0.855 |
| LGALS1 | ISG20 | 0.855 |
| DUSP6 | IFI44L | 0.855 |
| STXBP2 | MAFB | 0.855 |
| IFITM1 | BST2 | 0.855 |
| MAFB | HELZ2 | 0.855 |
| MAFB | SAMD9L | 0.855 |
| CD7 | IRF7 | 0.855 |
| IFI6 | TYMP | 0.855 |
| MAFB | CCDC190 | 0.855 |
| IFIT2 | PNMA1 | 0.855 |
| NKG7 | ISG20 | 0.855 |
| NKG7 | IFI44 | 0.855 |
| IFI6 | SAMD9L | 0.855 |
| LY6E | MX2 | 0.854 |
| IFI6 | TOR1B | 0.854 |
| LY6E | RSAD2 | 0.854 |
| IFIT2 | SHFL | 0.854 |
| MS4A6A | SAMD9L | 0.854 |
| STXBP2 | LGALS1 | 0.854 |
| NKG7 | CTSL | 0.854 |
| MAFB | FCER1G | 0.854 |
| MAFB | STAT2 | 0.854 |
| CD68 | IFI35 | 0.854 |
| IFIT1 | KLHDC7B | 0.854 |
| LY6E | ALDH3A1 | 0.854 |
| RTP4 | TSTD1 | 0.854 |
| STAT2 | INPP5E | 0.854 |
| IFIT1 | CCR1 | 0.854 |
| TMSB10 | INPP5E | 0.854 |
| TLNRD1 | OAS3 | 0.854 |
| C1QA | NKG7 | 0.854 |
| STXBP2 | HELZ2 | 0.854 |
| IFITM3 | PPP1R3D | 0.854 |
| FCER1G | KLHDC7B | 0.854 |
| TMUB2 | IFI44L | 0.854 |
| IFIT1 | DUSP6 | 0.854 |
| IFI35 | IFIT2 | 0.854 |
| C1QA | MX1 | 0.854 |
| SLC16A3 | RSAD2 | 0.854 |
| CDKN1C | MX1 | 0.854 |
| TMSB10 | FCER1G | 0.854 |
| IFITM2 | CD68 | 0.854 |
| ISG15 | OAS2 | 0.854 |
| SAMD9L | PRDX5 | 0.854 |
| IFITM1 | ISG20 | 0.854 |
| IFITM3 | OAS2 | 0.854 |
| LY6E | HLA-E | 0.854 |
| IFI6 | IFIT5 | 0.854 |
| IFITM1 | IFI44 | 0.854 |
| HLA-A | SHFL | 0.854 |
| SAMD9 | IFIT1 | 0.854 |
| MAFB | TNFSF10 | 0.853 |
| MS4A6A | CXCL10 | 0.853 |
| MS4A6A | RTP4 | 0.853 |
| C1QC | IFITM2 | 0.853 |
| H4C8 | NKG7 | 0.853 |
| IFITM1 | IFIT5 | 0.853 |
| MAFB | TMUB2 | 0.853 |
| SIGLEC10 | IFIT1 | 0.853 |
| OAS3 | SECTM1 | 0.853 |
| IFIT3 | PRDX5 | 0.853 |
| MS4A6A | C6orf47 | 0.853 |
| TLNRD1 | IFI44L | 0.853 |
| CDKN1C | CASP1 | 0.853 |
| TIMP1 | MAFB | 0.853 |
| LGALS1 | TMUB2 | 0.853 |
| FCER1G | IRF7 | 0.853 |
| IFITM2 | RTP4 | 0.853 |
| ISG15 | RTP4 | 0.853 |
| KLHDC7B | CCDC190 | 0.853 |
| C1QA | FCER1G | 0.853 |
| C1QA | IFI44 | 0.853 |
| HLA-B | OAS3 | 0.853 |
| IFITM2 | RSAD2 | 0.853 |
| IFITM3 | ATF5 | 0.853 |
| IFITM2 | MX1 | 0.853 |
| C1QC | IFI44L | 0.852 |
| TMSB10 | IRF7 | 0.852 |
| CTSL | LY6E | 0.852 |
| MAFB | OAS2 | 0.852 |
| TMEM199 | OAS3 | 0.852 |
| CTSL | IFI44L | 0.852 |
| IFITM3 | KLHDC7B | 0.852 |
| IFIT1 | IFI44L | 0.852 |
| MAFB | PRDX5 | 0.852 |
| STXBP2 | CXCL10 | 0.852 |
| TAP1 | XAF1 | 0.852 |
| IFITM3 | ISG20 | 0.852 |
| LY6E | IFIT5 | 0.852 |
| IFI35 | SECTM1 | 0.852 |
| CTSL | CCDC190 | 0.852 |
| MS4A6A | TNFSF10 | 0.852 |
| C1QC | NKG7 | 0.852 |
| CDKN1C | SAMD9 | 0.852 |
| C6orf47 | OAS3 | 0.852 |
| LY6E | PPP1R3D | 0.852 |
| IFIT2 | TSTD1 | 0.852 |
| MS4A6A | STXBP2 | 0.852 |
| NKG7 | TYMP | 0.852 |
| OAS1 | CD7 | 0.852 |
| XAF1 | IFI35 | 0.852 |
| MS4A6A | LGALS1 | 0.852 |
| NKG7 | SAMD9L | 0.852 |
| SERTAD1 | IFI35 | 0.852 |
| IFITM2 | MT2A | 0.852 |
| CXCL10 | GSTA1 | 0.852 |
| NKG7 | CCR1 | 0.852 |
| LY6E | HLA-F | 0.852 |
| IFIT1 | TNFSF10 | 0.852 |
| IFIT5 | GSTA1 | 0.852 |
| OAS3 | PNMA1 | 0.851 |
| FCER1G | SAMD9L | 0.851 |
| HLA-F | XAF1 | 0.851 |
| MS4A6A | SAMD9 | 0.851 |
| OAS1 | SECTM1 | 0.851 |
| HLA-A | IFIT3 | 0.851 |
| IFI6 | MT2A | 0.851 |
| TOR1B | IFI44L | 0.851 |
| IFIT1 | CASP1 | 0.851 |
| IFI44L | PPP1R3D | 0.851 |
| IFIT1 | ALDH3A1 | 0.851 |
| C1QC | LGALS1 | 0.851 |
| TIMP1 | SHFL | 0.851 |
| MAFB | HLA-E | 0.851 |
| MAFB | MX2 | 0.851 |
| OAS3 | IFIT2 | 0.851 |
| CD68 | IRF7 | 0.851 |
| IFIT1 | PPP1R3D | 0.851 |
| MX1 | PNMA1 | 0.851 |
| OAS1 | IFI44L | 0.851 |
| CD7 | RSAD2 | 0.851 |
| NADK | XAF1 | 0.851 |
| TYMP | XAF1 | 0.851 |
| RSAD2 | SECTM1 | 0.851 |
| HELZ2 | CCDC190 | 0.851 |
| IFIT3 | PNMA1 | 0.851 |
| SOCS1 | GSTA1 | 0.851 |
| STXBP2 | STAT2 | 0.851 |
| OAS1 | OAS3 | 0.851 |
| OAS1 | CD68 | 0.851 |
| C6orf47 | XAF1 | 0.851 |
| TIMP1 | RTP4 | 0.851 |
| MAFB | ISG20 | 0.851 |
| CD68 | IFI44L | 0.851 |
| IFIT1 | SAMD9L | 0.851 |
| IFI44L | CCR1 | 0.851 |
| IRF7 | PNMA1 | 0.851 |
| NKG7 | TOR1B | 0.851 |
| OAS1 | MT2A | 0.851 |
| LGALS1 | MX2 | 0.851 |
| MS4A6A | PNMA1 | 0.851 |
| OAS1 | PNMA1 | 0.851 |
| SLC16A3 | IFIT2 | 0.850 |
| CDKN1C | TIMP1 | 0.850 |
| OAS1 | SHFL | 0.850 |
| TAP1 | IFI44L | 0.850 |
| IFIT2 | TPRG1L | 0.850 |
| MAFB | NADK | 0.850 |
| MS4A6A | MX2 | 0.850 |
| NKG7 | BST2 | 0.850 |
| LGALS1 | HLA-E | 0.850 |
| NADK | IFI44L | 0.850 |
| IRF7 | SECTM1 | 0.850 |
| IRF7 | PRDX5 | 0.850 |
| C1QC | IFIT3 | 0.850 |
| C6orf47 | IFI44L | 0.850 |
| IFI6 | KLF6 | 0.850 |
| FCER1G | BST2 | 0.850 |
| TMUB2 | IFIT2 | 0.850 |
| IFIT1 | IFIT5 | 0.850 |
| NKG7 | TAP1 | 0.850 |
| OAS3 | HLA-F | 0.850 |
| OAS1 | IFI35 | 0.850 |
| CTSL | FCER1G | 0.850 |
| IFI35 | DUSP6 | 0.850 |
| IFIT5 | TSTD1 | 0.850 |
| MAFB | PNMA1 | 0.850 |
| SLC16A3 | IFI44 | 0.850 |
| OAS1 | RSAD2 | 0.850 |
| C6orf47 | MAFB | 0.850 |
| SIGLEC10 | RSAD2 | 0.850 |
| CDKN1C | HELZ2 | 0.850 |
| IFITM1 | SOCS1 | 0.850 |
| TLNRD1 | RSAD2 | 0.849 |
| TMEM199 | IFITM2 | 0.849 |
| NKG7 | NADK | 0.849 |
| IFITM1 | CXCL10 | 0.849 |
| HLA-A | MT2A | 0.849 |
| HLA-A | IFI44L | 0.849 |
| TAP1 | MT2A | 0.849 |
| IRF7 | IFI35 | 0.849 |
| RSAD2 | TPRG1L | 0.849 |
| HLA-B | MAFB | 0.849 |
| IFITM3 | IFI44 | 0.849 |
| IRF7 | IFIT2 | 0.849 |
| IFIT3 | INPP5E | 0.849 |
| IFI6 | IFI44L | 0.849 |
| IFI6 | TNFSF10 | 0.849 |
| IFI6 | CXCL10 | 0.849 |
| HELZ2 | GSTA1 | 0.849 |
| CTSL | MX2 | 0.849 |
| LGALS1 | RTP4 | 0.849 |
| IFIT1 | SOCS1 | 0.849 |
| IFIT2 | IFI44 | 0.849 |
| CDKN1C | IFIT5 | 0.849 |
| TIMP1 | TOR1B | 0.849 |
| CTSL | RSAD2 | 0.849 |
| IFITM3 | MX2 | 0.849 |
| LGALS1 | CXCL10 | 0.849 |
| TYMP | IFI44L | 0.849 |
| IFIT1 | IFIT3 | 0.849 |
| IFIT1 | GBP3 | 0.849 |
| RTP4 | INPP5E | 0.849 |
| TIMP1 | SAMD9 | 0.849 |
| SERTAD1 | OAS3 | 0.849 |
| CD7 | FCER1G | 0.849 |
| TMEM199 | XAF1 | 0.849 |
| IL2RG | IFIT1 | 0.848 |
| HLA-F | IRF7 | 0.848 |
| TMUB2 | OAS2 | 0.848 |
| IFIT1 | ATF5 | 0.848 |
| IFITM1 | EIF2AK2 | 0.848 |
| IFIT1 | STAT2 | 0.848 |
| CDKN1C | IFI44 | 0.848 |
| NKG7 | HLA-F | 0.848 |
| LY6E | MX1 | 0.848 |
| LGALS1 | INPP5E | 0.848 |
| SLC16A3 | SAMD9L | 0.848 |
| LY6E | MT2A | 0.848 |
| FCER1G | IFIT2 | 0.848 |
| IFITM2 | OAS2 | 0.848 |
| IFIT1 | EIF2AK2 | 0.848 |
| IFIT2 | ALDH3A1 | 0.848 |
| RTP4 | PNMA1 | 0.848 |
| MS4A6A | HLA-F | 0.848 |
| MS4A6A | CD68 | 0.848 |
| IFITM3 | BST2 | 0.848 |
| MAFB | INPP5E | 0.848 |
| IFIT2 | INPP5E | 0.848 |
| C1QA | TAP1 | 0.848 |
| C1QA | SAMD9L | 0.848 |
| H4C8 | RSAD2 | 0.848 |
| NKG7 | OAS2 | 0.848 |
| IFITM1 | NADK | 0.848 |
| IFITM1 | PPP1R3D | 0.848 |
| IFITM1 | RTP4 | 0.848 |
| SERTAD1 | RSAD2 | 0.848 |
| CD7 | IFI35 | 0.848 |
| IFI6 | KLHDC7B | 0.848 |
| MT2A | TSTD1 | 0.848 |
| CDKN1C | IFI35 | 0.848 |
| NKG7 | RTP4 | 0.848 |
| IFITM1 | CCR1 | 0.848 |
| SERTAD1 | IRF7 | 0.848 |
| IFITM2 | IFIT2 | 0.848 |
| CD68 | IFIT3 | 0.848 |
| IFIT3 | IFI35 | 0.848 |
| CD7 | IFI44L | 0.847 |
| LGALS1 | PARP14 | 0.847 |
| C1QC | OAS1 | 0.847 |
| HLA-B | NKG7 | 0.847 |
| TMSB10 | IFIT3 | 0.847 |
| TMEM199 | IFI44L | 0.847 |
| SLC16A3 | LGALS1 | 0.847 |
| TYMP | OAS3 | 0.847 |
| MT2A | SECTM1 | 0.847 |
| HLA-B | LY6E | 0.847 |
| HLA-B | XAF1 | 0.847 |
| TAP1 | IFI35 | 0.847 |
| IFITM3 | CXCL10 | 0.847 |
| IFI6 | EIF2AK2 | 0.847 |
| IFITM2 | TPRG1L | 0.847 |
| MS4A6A | SLC16A3 | 0.847 |
| MS4A6A | PARP14 | 0.847 |
| CD7 | IFIT3 | 0.847 |
| CD68 | SECTM1 | 0.847 |
| OAS2 | PPP1R3D | 0.847 |
| IFIT2 | RSAD2 | 0.847 |
| SAMD9 | CCDC190 | 0.847 |
| EIF2AK2 | PNMA1 | 0.847 |
| MAFB | IFIT5 | 0.847 |
| IFITM3 | SOCS1 | 0.847 |
| OAS3 | HLA-E | 0.847 |
| OAS3 | SHFL | 0.847 |
| CD68 | IFIT5 | 0.847 |
| C1QC | FCER1G | 0.847 |
| TMSB10 | IFI35 | 0.847 |
| LGALS1 | PARP9 | 0.847 |
| OAS3 | RSAD2 | 0.847 |
| IFIT1 | RTP4 | 0.847 |
| TAP1 | PRDX5 | 0.847 |
| MS4A6A | TIMP1 | 0.846 |
| NKG7 | TMEM199 | 0.846 |
| OAS1 | TMSB10 | 0.846 |
| IFITM1 | KLF6 | 0.846 |
| TIMP1 | BST2 | 0.846 |
| LGALS1 | NADK | 0.846 |
| LGALS1 | KLHDC7B | 0.846 |
| CD68 | XAF1 | 0.846 |
| IRF7 | MT2A | 0.846 |
| IFIT3 | MT2A | 0.846 |
| CXCL10 | CCDC190 | 0.846 |
| C1QA | TOR1B | 0.846 |
| NKG7 | SERTAD1 | 0.846 |
| CTSL | MX1 | 0.846 |
| CD7 | CD68 | 0.846 |
| IFI6 | MX2 | 0.846 |
| IFI35 | ISG20 | 0.846 |
| STXBP2 | IFIT5 | 0.846 |
| STXBP2 | MT2A | 0.846 |
| NADK | RSAD2 | 0.846 |
| OAS3 | CASP1 | 0.846 |
| TMUB2 | MT2A | 0.846 |
| STAT2 | GSTA1 | 0.846 |
| SLC16A3 | MAFB | 0.846 |
| MAFB | TYMP | 0.846 |
| MAFB | CXCL10 | 0.846 |
| CD7 | IFITM2 | 0.846 |
| TMEM199 | IFIT3 | 0.846 |
| IFITM2 | STAT2 | 0.846 |
| IFIT1 | BST2 | 0.846 |
| MX1 | TSTD1 | 0.846 |
| BST2 | PRDX5 | 0.846 |
| H4C8 | IFI44L | 0.846 |
| SLC16A3 | SHFL | 0.846 |
| IFITM1 | MX2 | 0.846 |
| LGALS1 | SOCS1 | 0.846 |
| IFI6 | IFI44 | 0.846 |
| FCER1G | IFIT3 | 0.846 |
| IRF7 | SHFL | 0.846 |
| IFIT1 | ISG20 | 0.846 |
| IFIT1 | IFI44 | 0.846 |
| OAS3 | TPRG1L | 0.846 |
| TLNRD1 | OAS1 | 0.846 |
| HLA-A | MX1 | 0.846 |
| IFI6 | ATF5 | 0.846 |
| OAS3 | MT2A | 0.846 |
| LGALS1 | TSTD1 | 0.846 |
| MS4A6A | PRDX5 | 0.846 |
| SAMD9L | INPP5E | 0.846 |
| C1QA | CXCL10 | 0.846 |
| NKG7 | TMSB10 | 0.846 |
| LGALS1 | TMEM199 | 0.846 |
| OAS3 | DUSP6 | 0.846 |
| IFIT3 | XAF1 | 0.846 |
| SAMD9 | PNMA1 | 0.846 |
| XAF1 | SHFL | 0.846 |
| IFITM2 | IFIT5 | 0.845 |
| CDKN1C | CXCL10 | 0.845 |
| IFITM1 | TOR1B | 0.845 |
| IFITM3 | CCR1 | 0.845 |
| LY6E | SAMD9L | 0.845 |
| LY6E | IFI44L | 0.845 |
| IFI35 | IFI44L | 0.845 |
| NKG7 | CXCL10 | 0.845 |
| TMSB10 | IFI44L | 0.845 |
| TOR1B | IFIT1 | 0.845 |
| IFITM2 | SAMD9L | 0.845 |
| OAS3 | PPP1R3D | 0.845 |
| IFIT1 | CXCL10 | 0.845 |
| MS4A6A | TMUB2 | 0.845 |
| H4C8 | OAS3 | 0.845 |
| SLC16A3 | OAS2 | 0.845 |
| TYMP | RSAD2 | 0.845 |
| FCER1G | IFIT5 | 0.845 |
| FCER1G | STAT2 | 0.845 |
| IFI44L | ALDH3A1 | 0.845 |
| SLC16A3 | STAT2 | 0.845 |
| CDKN1C | LY6E | 0.845 |
| C6orf47 | LY6E | 0.845 |
| TIMP1 | TRIM22 | 0.845 |
| MAFB | KLHDC7B | 0.845 |
| SAMD9 | LY6E | 0.845 |
| IFITM3 | EIF2AK2 | 0.845 |
| LY6E | CXCL10 | 0.845 |
| IFIT5 | IFIT2 | 0.845 |
| IFI35 | PPP1R3D | 0.845 |
| IRF7 | TPRG1L | 0.845 |
| MS4A6A | HLA-B | 0.845 |
| IFITM1 | PARP14 | 0.845 |
| TMSB10 | HLA-A | 0.845 |
| SERTAD1 | MAFB | 0.845 |
| FCER1G | EIF2AK2 | 0.845 |
| XAF1 | TPRG1L | 0.845 |
| STXBP2 | IFI44 | 0.845 |
| MAFB | TSTD1 | 0.845 |
| ISG20 | GSTA1 | 0.845 |
| OAS3 | IFIT3 | 0.844 |
| IFIT1 | MX1 | 0.844 |
| IFITM3 | RTP4 | 0.844 |
| HLA-F | IFI35 | 0.844 |
| SHFL | PRDX5 | 0.844 |
| IFI6 | SOCS1 | 0.844 |
| SIGLEC10 | IFI44 | 0.844 |
| IFI6 | TRIM22 | 0.844 |
| OAS3 | HELZ2 | 0.844 |
| DUSP6 | RSAD2 | 0.844 |
| C1QC | TIMP1 | 0.844 |
| OAS1 | MX1 | 0.844 |
| OAS3 | IFI44L | 0.844 |
| IRF7 | RSAD2 | 0.844 |
| IFIT1 | OAS2 | 0.844 |
| XAF1 | ISG20 | 0.844 |
| IFIT2 | MX1 | 0.844 |
| RSAD2 | CXCL10 | 0.844 |
| CTSL | PNMA1 | 0.844 |
| IFIT5 | PRDX5 | 0.844 |
| STXBP2 | MX2 | 0.844 |
| HLA-B | IRF7 | 0.844 |
| TIMP1 | HELZ2 | 0.844 |
| IFITM2 | HELZ2 | 0.844 |
| CD68 | MX1 | 0.844 |
| HELZ2 | IFIT2 | 0.844 |
| NKG7 | GSTA1 | 0.844 |
| TLNRD1 | IFIT3 | 0.844 |
| HLA-B | OAS1 | 0.844 |
| IFITM1 | HLA-E | 0.844 |
| TIMP1 | PARP14 | 0.844 |
| IRF7 | CXCL10 | 0.844 |
| HELZ2 | IFI44L | 0.844 |
| NKG7 | TRIM22 | 0.844 |
| C6orf47 | LGALS1 | 0.844 |
| HLA-A | SAMD9L | 0.844 |
| TOR1B | IFI35 | 0.844 |
| MT2A | RSAD2 | 0.844 |
| HLA-F | RSAD2 | 0.844 |
| IFIT3 | IFI44L | 0.844 |
| H4C8 | XAF1 | 0.843 |
| OAS1 | CTSL | 0.843 |
| IFI6 | OAS2 | 0.843 |
| C1QC | MX1 | 0.843 |
| IFI35 | RSAD2 | 0.843 |
| HLA-B | LGALS1 | 0.843 |
| NKG7 | CASP1 | 0.843 |
| LGALS1 | FCER1G | 0.843 |
| XAF1 | MT2A | 0.843 |
| IFIT2 | CXCL10 | 0.843 |
| SAMD9L | TSTD1 | 0.843 |
| IFITM1 | GBP3 | 0.843 |
| CTSL | ISG20 | 0.843 |
| IFITM3 | CASP1 | 0.843 |
| IFIT3 | SHFL | 0.843 |
| ISG20 | IFI44L | 0.843 |
| TOR1B | PNMA1 | 0.843 |
| MS4A6A | TOR1B | 0.843 |
| SERTAD1 | IFIT2 | 0.843 |
| TMUB2 | IFIT3 | 0.843 |
| IFI44L | RSAD2 | 0.843 |
| C1QC | SAMD9L | 0.843 |
| HLA-B | IFI44L | 0.843 |
| IFITM1 | DUSP6 | 0.843 |
| C6orf47 | FCER1G | 0.843 |
| MAFB | TMEM199 | 0.843 |
| IFITM3 | PARP14 | 0.843 |
| FCER1G | TNFSF10 | 0.843 |
| HLA-F | IFI44L | 0.843 |
| CD68 | RSAD2 | 0.843 |
| HELZ2 | XAF1 | 0.843 |
| OAS2 | IFIT2 | 0.843 |
| IFIT2 | RTP4 | 0.843 |
| IFI44L | CASP1 | 0.843 |
| C1QA | SHFL | 0.843 |
| TMSB10 | RSAD2 | 0.843 |
| MAFB | RTP4 | 0.843 |
| FCER1G | CXCL10 | 0.843 |
| OAS3 | MX1 | 0.843 |
| XAF1 | DUSP6 | 0.843 |
| ISG20 | PRDX5 | 0.843 |
| HELZ2 | INPP5E | 0.843 |
| TLNRD1 | HLA-A | 0.842 |
| MAFB | TOR1B | 0.842 |
| OAS3 | ISG20 | 0.842 |
| IRF7 | KLHDC7B | 0.842 |
| XAF1 | RSAD2 | 0.842 |
| NKG7 | ALDH3A1 | 0.842 |
| OAS1 | TYMP | 0.842 |
| OAS1 | HLA-F | 0.842 |
| TOR1B | RSAD2 | 0.842 |
| TLNRD1 | GSTA1 | 0.842 |
| H4C8 | FCER1G | 0.842 |
| H4C8 | CXCL10 | 0.842 |
| SLC16A3 | IFIT3 | 0.842 |
| MAFB | TRIM22 | 0.842 |
| LY6E | BST2 | 0.842 |
| LGALS1 | CASP1 | 0.842 |
| SERTAD1 | XAF1 | 0.842 |
| STXBP2 | TMSB10 | 0.842 |
| CD68 | SAMD9L | 0.842 |
| KLF6 | IFI44L | 0.842 |
| OAS1 | BST2 | 0.842 |
| SIGLEC10 | OAS2 | 0.842 |
| OAS3 | EIF2AK2 | 0.842 |
| XAF1 | IFI44L | 0.842 |
| SAMD9L | IFIT2 | 0.842 |
| IFIT2 | SECTM1 | 0.842 |
| C1QA | TRIM22 | 0.842 |
| HLA-B | IFIT2 | 0.842 |
| SERTAD1 | MT2A | 0.842 |
| MAFB | PPP1R3D | 0.842 |
| TAP1 | CD68 | 0.842 |
| HLA-F | IFIT2 | 0.842 |
| IRF7 | IFIT3 | 0.842 |
| IFI35 | HLA-E | 0.842 |
| MS4A6A | SERTAD1 | 0.842 |
| HLA-B | RSAD2 | 0.842 |
| NKG7 | TMUB2 | 0.842 |
| TMSB10 | TOR1B | 0.842 |
| IFITM2 | IFIT3 | 0.842 |
| OAS3 | IFI35 | 0.842 |
| RSAD2 | PPP1R3D | 0.842 |
| LGALS1 | CCDC190 | 0.842 |
| NKG7 | TSTD1 | 0.842 |
| IFI44L | CXCL10 | 0.842 |
| C1QA | IFIT5 | 0.841 |
| C1QA | IFI35 | 0.841 |
| C1QA | ISG20 | 0.841 |
| STXBP2 | TAP1 | 0.841 |
| NKG7 | SOCS1 | 0.841 |
| FCER1G | HELZ2 | 0.841 |
| GBP3 | CCDC190 | 0.841 |
| HLA-A | TPRG1L | 0.841 |
| IFITM2 | PRDX5 | 0.841 |
| CASP1 | PRDX5 | 0.841 |
| LY6E | EIF2AK2 | 0.841 |
| RIPK3 | IFI44L | 0.841 |
| IFIT1 | KLF6 | 0.841 |
| OAS2 | DUSP6 | 0.841 |
| IFIT2 | KLHDC7B | 0.841 |
| RSAD2 | CCR1 | 0.841 |
| SLC16A3 | TNFSF10 | 0.841 |
| STXBP2 | SECTM1 | 0.841 |
| MAFB | CD68 | 0.841 |
| TAP1 | RSAD2 | 0.841 |
| IFITM3 | TOR1B | 0.841 |
| IFITM3 | HLA-E | 0.841 |
| LY6E | OAS2 | 0.841 |
| TMEM199 | MX1 | 0.841 |
| IFIT2 | ATF5 | 0.841 |
| NKG7 | PPP1R3D | 0.841 |
| IFIT3 | RSAD2 | 0.841 |
| IFIT2 | STAT2 | 0.841 |
| IFITM1 | CASP1 | 0.841 |
| OAS3 | MX2 | 0.841 |
| IRF7 | BST2 | 0.841 |
| TMUB2 | IFI44 | 0.841 |
| MS4A6A | ALDH3A1 | 0.841 |
| RSAD2 | ALDH3A1 | 0.841 |
| TAP1 | PNMA1 | 0.841 |
| PARP14 | GSTA1 | 0.841 |
| C1QA | HELZ2 | 0.841 |
| IFIT5 | IFI44L | 0.841 |
| IFITM2 | TSTD1 | 0.841 |
| C1QC | IFI44 | 0.841 |
| H4C8 | IFITM2 | 0.841 |
| C6orf47 | IFIT2 | 0.841 |
| SERTAD1 | LGALS1 | 0.841 |
| HLA-A | HELZ2 | 0.841 |
| CD68 | MT2A | 0.841 |
| BST2 | RSAD2 | 0.841 |
| CCR1 | IFI44 | 0.841 |
| IFIT3 | ALDH3A1 | 0.841 |
| OAS1 | TMEM199 | 0.840 |
| OAS1 | IFIT3 | 0.840 |
| TAP1 | OAS3 | 0.840 |
| CD68 | HELZ2 | 0.840 |
| XAF1 | HLA-E | 0.840 |
| TLNRD1 | CCDC190 | 0.840 |
| TAP1 | TSTD1 | 0.840 |
| NADK | IFIT2 | 0.840 |
| IFITM1 | TRIM22 | 0.840 |
| C6orf47 | RSAD2 | 0.840 |
| CXCL10 | PRDX5 | 0.840 |
| CDKN1C | EIF2AK2 | 0.840 |
| TMUB2 | ISG20 | 0.840 |
| STXBP2 | SHFL | 0.840 |
| CDKN1C | MX2 | 0.840 |
| CTSL | BST2 | 0.840 |
| IRF7 | MX1 | 0.840 |
| MX2 | RSAD2 | 0.840 |
| TMSB10 | PNMA1 | 0.840 |
| C1QC | GSTA1 | 0.840 |
| C6orf47 | MX1 | 0.840 |
| IFITM3 | GBP3 | 0.840 |
| IFI6 | PARP14 | 0.840 |
| OAS3 | IFIT5 | 0.840 |
| IFIT1 | TRIM22 | 0.840 |
| IFI35 | MX1 | 0.840 |
| ISG20 | CCDC190 | 0.840 |
| MS4A6A | C1QA | 0.840 |
| TLNRD1 | BST2 | 0.840 |
| C1QC | ISG20 | 0.840 |
| TOR1B | XAF1 | 0.840 |
| TAP1 | TPRG1L | 0.840 |
| IFIT3 | TPRG1L | 0.840 |
| LGALS1 | PNMA1 | 0.840 |
| MS4A6A | C1QC | 0.840 |
| CDKN1C | ISG20 | 0.840 |
| HLA-B | MT2A | 0.840 |
| TMSB10 | CD68 | 0.840 |
| IFI6 | STAT2 | 0.840 |
| FCER1G | CD68 | 0.840 |
| ISG15 | GBP1 | 0.840 |
| TLNRD1 | OAS2 | 0.839 |
| TIMP1 | ISG20 | 0.839 |
| SAMD9 | FCER1G | 0.839 |
| IFITM2 | TNFSF10 | 0.839 |
| IRF7 | TNFSF10 | 0.839 |
| BST2 | PNMA1 | 0.839 |
| TNFSF10 | PNMA1 | 0.839 |
| TIMP1 | CD7 | 0.839 |
| NADK | IRF7 | 0.839 |
| TYMP | IFI44 | 0.839 |
| NKG7 | KLHDC7B | 0.839 |
| OAS1 | HELZ2 | 0.839 |
| CTSL | LGALS1 | 0.839 |
| LY6E | CCR1 | 0.839 |
| NADK | IFIT3 | 0.839 |
| FCER1G | SOCS1 | 0.839 |
| TMUB2 | SAMD9L | 0.839 |
| IFIT2 | TNFSF10 | 0.839 |
| HLA-E | MT2A | 0.839 |
| SAMD9 | PRDX5 | 0.839 |
| LGALS1 | PRDX5 | 0.839 |
| C1QA | OAS2 | 0.839 |
| C6orf47 | IFI35 | 0.839 |
| SHFL | PNMA1 | 0.839 |
| C1QC | HLA-A | 0.839 |
| IL2RG | LY6E | 0.839 |
| IL2RG | OAS3 | 0.839 |
| SAMD9 | IRF7 | 0.839 |
| CD68 | OAS2 | 0.839 |
| IFIT1 | MX2 | 0.839 |
| IFIT2 | EIF2AK2 | 0.839 |
| SECTM1 | IFI44 | 0.839 |
| ISG20 | INPP5E | 0.839 |
| C1QA | MX2 | 0.839 |
| TIMP1 | LGALS1 | 0.839 |
| OAS3 | CCR1 | 0.839 |
| OAS3 | KLHDC7B | 0.839 |
| XAF1 | PPP1R3D | 0.839 |
| MX2 | IFI44L | 0.839 |
| NKG7 | TNFSF10 | 0.839 |
| IL2RG | OAS2 | 0.839 |
| IL2RG | RSAD2 | 0.839 |
| HLA-A | IFI44 | 0.839 |
| FCER1G | GBP3 | 0.839 |
| OAS3 | TNFSF10 | 0.839 |
| IRF7 | IFI44 | 0.839 |
| IFI44 | CASP1 | 0.839 |
| SAMD9 | XAF1 | 0.838 |
| IFITM3 | TRIM22 | 0.838 |
| IFI6 | MX1 | 0.838 |
| IRF7 | ISG20 | 0.838 |
| IFI44L | TNFSF10 | 0.838 |
| TAP1 | CCDC190 | 0.838 |
| MS4A6A | NADK | 0.838 |
| TAP1 | IRF7 | 0.838 |
| IFIT2 | BST2 | 0.838 |
| IFIT3 | TSTD1 | 0.838 |
| HLA-B | IFI35 | 0.838 |
| NKG7 | HLA-E | 0.838 |
| TYMP | IRF7 | 0.838 |
| XAF1 | CXCL10 | 0.838 |
| MS4A6A | CASP1 | 0.838 |
| CD68 | SHFL | 0.838 |
| IFIT5 | CXCL10 | 0.838 |
| IFIT2 | SOCS1 | 0.838 |
| HLA-E | RSAD2 | 0.838 |
| OAS1 | TPRG1L | 0.838 |
| C1QC | MX2 | 0.838 |
| TYMP | MT2A | 0.838 |
| XAF1 | CCR1 | 0.838 |
| XAF1 | MX1 | 0.838 |
| IFI35 | CCR1 | 0.838 |
| DUSP6 | MX1 | 0.838 |
| OAS1 | ALDH3A1 | 0.838 |
| KLHDC7B | GSTA1 | 0.838 |
| C1QC | IFI35 | 0.838 |
| OAS1 | ISG20 | 0.838 |
| TMSB10 | TYMP | 0.838 |
| TMSB10 | IFIT5 | 0.838 |
| TAP1 | FCER1G | 0.838 |
| CD7 | SAMD9L | 0.838 |
| IFI35 | ALDH3A1 | 0.838 |
| TAP1 | GSTA1 | 0.838 |
| C1QA | MT2A | 0.837 |
| H4C8 | OAS1 | 0.837 |
| IFIT5 | SECTM1 | 0.837 |
| FCER1G | TSTD1 | 0.837 |
| C1QA | TMSB10 | 0.837 |
| OAS1 | CXCL10 | 0.837 |
| TYMP | IFIT3 | 0.837 |
| RSAD2 | CASP1 | 0.837 |
| IFIT5 | INPP5E | 0.837 |
| OAS1 | TAP1 | 0.837 |
| MS4A6A | ATF5 | 0.837 |
| TOR1B | OAS2 | 0.837 |
| ISG20 | RSAD2 | 0.837 |
| CTSL | PRDX5 | 0.837 |
| CXCL10 | INPP5E | 0.837 |
| MS4A6A | TRIM22 | 0.837 |
| NKG7 | STAT2 | 0.837 |
| OAS1 | KLHDC7B | 0.837 |
| TIMP1 | HLA-F | 0.837 |
| CTSL | CXCL10 | 0.837 |
| TMEM199 | FCER1G | 0.837 |
| IFITM2 | CXCL10 | 0.837 |
| OAS3 | BST2 | 0.837 |
| HELZ2 | IFI35 | 0.837 |
| IFIT3 | SECTM1 | 0.837 |
| DUSP6 | MT2A | 0.837 |
| EIF2AK2 | INPP5E | 0.837 |
| TLNRD1 | MX1 | 0.837 |
| H4C8 | IRF7 | 0.837 |
| H4C8 | IFIT2 | 0.837 |
| STXBP2 | FCER1G | 0.837 |
| TMSB10 | MX2 | 0.837 |
| TAP1 | IFI44 | 0.837 |
| CD7 | SHFL | 0.837 |
| LY6E | TNFSF10 | 0.837 |
| CXCL10 | ALDH3A1 | 0.837 |
| TYMP | PRDX5 | 0.837 |
| TLNRD1 | TIMP1 | 0.837 |
| SERTAD1 | IFIT3 | 0.837 |
| TYMP | IFI35 | 0.837 |
| TMEM199 | RSAD2 | 0.837 |
| OAS3 | SAMD9L | 0.837 |
| CD68 | TNFSF10 | 0.837 |
| IFIT1 | PARP14 | 0.837 |
| SECTM1 | SHFL | 0.837 |
| CTSL | MAFB | 0.837 |
| SAMD9 | CD7 | 0.837 |
| CD7 | IFI44 | 0.837 |
| TYMP | IFIT2 | 0.837 |
| IRF7 | IFIT5 | 0.837 |
| IFI35 | BST2 | 0.837 |
| IFI44L | TPRG1L | 0.837 |
| C1QC | TMSB10 | 0.836 |
| C1QC | SHFL | 0.836 |
| CDKN1C | TYMP | 0.836 |
| CTSL | SAMD9 | 0.836 |
| MAFB | SOCS1 | 0.836 |
| MAFB | GBP3 | 0.836 |
| TOR1B | CXCL10 | 0.836 |
| OAS2 | CASP1 | 0.836 |
| TOR1B | TPRG1L | 0.836 |
| IFITM2 | PNMA1 | 0.836 |
| C1QA | EIF2AK2 | 0.836 |
| IRF7 | MX2 | 0.836 |
| IFI35 | MT2A | 0.836 |
| MS4A6A | CCR1 | 0.836 |
| C1QA | TIMP1 | 0.836 |
| TAP1 | IFIT2 | 0.836 |
| TMEM199 | IFI35 | 0.836 |
| FCER1G | SECTM1 | 0.836 |
| IRF7 | EIF2AK2 | 0.836 |
| TMUB2 | HELZ2 | 0.836 |
| HELZ2 | IFIT3 | 0.836 |
| RSAD2 | MX1 | 0.836 |
| FCER1G | TPRG1L | 0.836 |
| C1QA | GSTA1 | 0.836 |
| TLNRD1 | CXCL10 | 0.836 |
| C1QA | CCR1 | 0.836 |
| STXBP2 | TNFSF10 | 0.836 |
| SAMD9 | OAS3 | 0.836 |
| LY6E | TRIM22 | 0.836 |
| NADK | SHFL | 0.836 |
| CD68 | STAT2 | 0.836 |
| TMUB2 | BST2 | 0.836 |
| IFIT3 | CXCL10 | 0.836 |
| MT2A | CXCL10 | 0.836 |
| C1QA | CCDC190 | 0.836 |
| MS4A6A | CTSL | 0.836 |
| TIMP1 | CXCL10 | 0.836 |
| HLA-A | CD68 | 0.836 |
| HLA-F | IFIT3 | 0.836 |
| SAMD9L | CXCL10 | 0.836 |
| IFI44L | EIF2AK2 | 0.836 |
| SECTM1 | MX1 | 0.836 |
| APOBEC3G | GSTA1 | 0.836 |
| CTSL | SECTM1 | 0.836 |
| MAFB | CASP1 | 0.836 |
| RIPK3 | IFIT2 | 0.836 |
| OAS3 | ALDH3A1 | 0.836 |
| TLNRD1 | PRDX5 | 0.836 |
| HELZ2 | PRDX5 | 0.836 |
| NKG7 | C6orf47 | 0.835 |
| MAFB | CCR1 | 0.835 |
| HLA-B | MX1 | 0.835 |
| TIMP1 | HLA-A | 0.835 |
| IFITM3 | DUSP6 | 0.835 |
| OAS3 | ATF5 | 0.835 |
| TLNRD1 | C1QA | 0.835 |
| PARP14 | CCDC190 | 0.835 |
| MAFB | PARP14 | 0.835 |
| IFITM3 | KLF6 | 0.835 |
| OAS3 | RIPK3 | 0.835 |
| CD68 | CXCL10 | 0.835 |
| IFIT3 | IFIT2 | 0.835 |
| DUSP6 | IFI44 | 0.835 |
| MT2A | CASP1 | 0.835 |
| CD68 | GSTA1 | 0.835 |
| SLC16A3 | EIF2AK2 | 0.835 |
| SERTAD1 | MX1 | 0.835 |
| CD7 | OAS2 | 0.835 |
| IFITM2 | BST2 | 0.835 |
| IFIT3 | ATF5 | 0.835 |
| XAF1 | SAMD9L | 0.835 |
| MAFB | TPRG1L | 0.835 |
| CXCL10 | PNMA1 | 0.835 |
| OAS1 | CASP1 | 0.835 |
| CTSL | OAS2 | 0.835 |
| CTSL | MT2A | 0.835 |
| C1QC | CCDC190 | 0.835 |
| ATF5 | GSTA1 | 0.835 |
| STXBP2 | TYMP | 0.835 |
| TMSB10 | SECTM1 | 0.835 |
| TIMP1 | GBP3 | 0.835 |
| SAMD9 | IFI44L | 0.835 |
| CD7 | MT2A | 0.835 |
| TMUB2 | SHFL | 0.835 |
| HELZ2 | RSAD2 | 0.835 |
| SAMD9L | IFI35 | 0.835 |
| TMSB10 | TPRG1L | 0.835 |
| C1QA | STXBP2 | 0.835 |
| CDKN1C | BST2 | 0.835 |
| OAS1 | STAT2 | 0.835 |
| LY6E | STAT2 | 0.835 |
| LY6E | IFI44 | 0.835 |
| NADK | OAS2 | 0.835 |
| OAS2 | CCR1 | 0.835 |
| ISG20 | TSTD1 | 0.835 |
| C1QC | HELZ2 | 0.834 |
| SLC16A3 | CD7 | 0.834 |
| HLA-A | EIF2AK2 | 0.834 |
| TAP1 | TMEM199 | 0.834 |
| TYMP | CXCL10 | 0.834 |
| TOR1B | MT2A | 0.834 |
| C1QC | CXCL10 | 0.834 |
| IL2RG | IFI44 | 0.834 |
| CD7 | CCR1 | 0.834 |
| TIMP1 | IFITM2 | 0.834 |
| TYMP | SHFL | 0.834 |
| OAS3 | CXCL10 | 0.834 |
| CD68 | IFI44 | 0.834 |
| IFI35 | CXCL10 | 0.834 |
| RSAD2 | EIF2AK2 | 0.834 |
| RSAD2 | SHFL | 0.834 |
| TMSB10 | OAS2 | 0.834 |
| TYMP | OAS2 | 0.834 |
| IFIT5 | MT2A | 0.834 |
| PPP1R3D | MX1 | 0.834 |
| SOCS1 | CCDC190 | 0.834 |
| TLNRD1 | SAMD9L | 0.834 |
| TLNRD1 | ISG20 | 0.834 |
| STXBP2 | SOCS1 | 0.834 |
| C6orf47 | IFIT3 | 0.834 |
| TAP1 | IFITM2 | 0.834 |
| NADK | MT2A | 0.834 |
| IFI44L | KLHDC7B | 0.834 |
| PPP1R3D | CXCL10 | 0.834 |
| TLNRD1 | SLC16A3 | 0.834 |
| TLNRD1 | SECTM1 | 0.834 |
| SLC16A3 | CXCL10 | 0.834 |
| OAS1 | PPP1R3D | 0.834 |
| SERTAD1 | IFI44 | 0.834 |
| SAMD9 | SECTM1 | 0.834 |
| IFI6 | GBP3 | 0.834 |
| TMEM199 | SAMD9L | 0.834 |
| OAS3 | OAS2 | 0.834 |
| XAF1 | TRIM22 | 0.834 |
| C1QC | TYMP | 0.833 |
| OAS1 | IFIT5 | 0.833 |
| TMSB10 | SAMD9 | 0.833 |
| SERTAD1 | OAS2 | 0.833 |
| LY6E | RIPK3 | 0.833 |
| SIGLEC10 | XAF1 | 0.833 |
| IRF7 | CCR1 | 0.833 |
| STAT2 | PNMA1 | 0.833 |
| NADK | MX1 | 0.833 |
| ISG20 | SHFL | 0.833 |
| OAS1 | EIF2AK2 | 0.833 |
| SAMD9 | CD68 | 0.833 |
| BST2 | INPP5E | 0.833 |
| CDKN1C | CTSL | 0.833 |
| TMSB10 | SAMD9L | 0.833 |
| IL2RG | IFI35 | 0.833 |
| TMEM199 | OAS2 | 0.833 |
| IFIT3 | BST2 | 0.833 |
| IFIT3 | IFI44 | 0.833 |
| IFI35 | MX2 | 0.833 |
| SOCS1 | IFI44L | 0.833 |
| CCR1 | SHFL | 0.833 |
| STXBP2 | SAMD9 | 0.833 |
| NKG7 | PARP14 | 0.833 |
| SAMD9 | RSAD2 | 0.833 |
| TYMP | SAMD9L | 0.833 |
| IRF7 | HELZ2 | 0.833 |
| IRF7 | SAMD9L | 0.833 |
| IFIT2 | ISG20 | 0.833 |
| GBP3 | GSTA1 | 0.833 |
| EIF2AK2 | GSTA1 | 0.833 |
| CDKN1C | IL2RG | 0.833 |
| OAS3 | TRIM22 | 0.833 |
| IRF7 | STAT2 | 0.833 |
| MT2A | ISG20 | 0.833 |
| SECTM1 | GSTA1 | 0.833 |
| TAP1 | INPP5E | 0.833 |
| TLNRD1 | TAP1 | 0.833 |
| CDKN1C | NADK | 0.833 |
| TMSB10 | CXCL10 | 0.833 |
| MAFB | PARP9 | 0.833 |
| LGALS1 | CD68 | 0.833 |
| HLA-E | IFI44 | 0.833 |
| RSAD2 | ATF5 | 0.833 |
| TYMP | TPRG1L | 0.833 |
| IFIT5 | TPRG1L | 0.833 |
| EIF2AK2 | TSTD1 | 0.833 |
| C1QA | HLA-F | 0.832 |
| C1QA | STAT2 | 0.832 |
| CDKN1C | STAT2 | 0.832 |
| OAS1 | IFI44 | 0.832 |
| SAMD9 | IFIT2 | 0.832 |
| SAMD9L | SECTM1 | 0.832 |
| SAMD9 | GSTA1 | 0.832 |
| OAS1 | C6orf47 | 0.832 |
| MS4A6A | HLA-E | 0.832 |
| SERTAD1 | CXCL10 | 0.832 |
| LY6E | SIGLEC10 | 0.832 |
| LGALS1 | DUSP6 | 0.832 |
| IFI6 | RTP4 | 0.832 |
| XAF1 | KLHDC7B | 0.832 |
| FCER1G | CCDC190 | 0.832 |
| FCER1G | PRDX5 | 0.832 |
| TLNRD1 | IFI35 | 0.832 |
| C1QA | BST2 | 0.832 |
| IL2RG | MT2A | 0.832 |
| IFITM2 | EIF2AK2 | 0.832 |
| TNFSF10 | RSAD2 | 0.832 |
| TLNRD1 | IFI44 | 0.832 |
| TMSB10 | TAP1 | 0.832 |
| XAF1 | IFIT5 | 0.832 |
| TMSB10 | ALDH3A1 | 0.832 |
| TLNRD1 | INPP5E | 0.832 |
| TLNRD1 | MT2A | 0.832 |
| TLNRD1 | CCR1 | 0.832 |
| C1QC | CD68 | 0.832 |
| IRF7 | OAS2 | 0.832 |
| IFIT2 | MX2 | 0.832 |
| MS4A6A | TPRG1L | 0.832 |
| MX1 | TPRG1L | 0.832 |
| SAMD9 | TSTD1 | 0.832 |
| C1QC | MT2A | 0.832 |
| OAS1 | OAS2 | 0.832 |
| CTSL | IFI44 | 0.832 |
| HELZ2 | CXCL10 | 0.832 |
| IFIT3 | MX1 | 0.832 |
| XAF1 | SOCS1 | 0.832 |
| OAS2 | RSAD2 | 0.832 |
| TRIM22 | PRDX5 | 0.832 |
| C1QC | IFIT5 | 0.832 |
| SLC16A3 | HELZ2 | 0.832 |
| MAFB | ATF5 | 0.832 |
| CD7 | HELZ2 | 0.832 |
| IFI6 | PARP9 | 0.832 |
| TYMP | CD68 | 0.832 |
| XAF1 | BST2 | 0.832 |
| STXBP2 | HLA-F | 0.831 |
| STXBP2 | RTP4 | 0.831 |
| CD7 | IFIT5 | 0.831 |
| SAMD9L | MT2A | 0.831 |
| MX1 | ALDH3A1 | 0.831 |
| TYMP | MX1 | 0.831 |
| RIPK3 | RSAD2 | 0.831 |
| XAF1 | ATF5 | 0.831 |
| CD68 | BST2 | 0.831 |
| XAF1 | CASP1 | 0.831 |
| IFITM2 | CCDC190 | 0.831 |
| SIGLEC10 | CXCL10 | 0.831 |
| SLC16A3 | RTP4 | 0.831 |
| OAS1 | CCR1 | 0.831 |
| IL2RG | XAF1 | 0.831 |
| LY6E | PARP9 | 0.831 |
| NADK | CXCL10 | 0.831 |
| TMEM199 | HELZ2 | 0.831 |
| TMUB2 | IFIT5 | 0.831 |
| CXCL10 | EIF2AK2 | 0.831 |
| TRIM22 | GSTA1 | 0.831 |
| SLC16A3 | BST2 | 0.831 |
| SAMD9 | MT2A | 0.831 |
| SAMD9 | CXCL10 | 0.831 |
| SIGLEC10 | IFI35 | 0.831 |
| XAF1 | TNFSF10 | 0.831 |
| ISG15 | APOBEC3G | 0.831 |
| OAS2 | HLA-E | 0.831 |
| MX2 | CXCL10 | 0.831 |
| LGALS1 | TPRG1L | 0.831 |
| STAT2 | PRDX5 | 0.831 |
| C1QA | CD68 | 0.831 |
| CTSL | EIF2AK2 | 0.831 |
| LY6E | KLF6 | 0.831 |
| HELZ2 | MT2A | 0.831 |
| IFI35 | CASP1 | 0.831 |
| RSAD2 | IFI44 | 0.831 |
| H4C8 | MX1 | 0.831 |
| IFITM1 | IL2RG | 0.831 |
| C6orf47 | IFI44 | 0.831 |
| SERTAD1 | SAMD9L | 0.831 |
| TAP1 | OAS2 | 0.831 |
| CD7 | MX1 | 0.831 |
| OAS3 | STAT2 | 0.831 |
| RIPK3 | XAF1 | 0.831 |
| MT2A | CCR1 | 0.831 |
| SHFL | TSTD1 | 0.831 |
| H4C8 | OAS2 | 0.830 |
| NKG7 | PARP9 | 0.830 |
| HLA-A | OAS2 | 0.830 |
| TAP1 | CXCL10 | 0.830 |
| IFI44L | TRIM22 | 0.830 |
| NKG7 | TPRG1L | 0.830 |
| OAS1 | SERTAD1 | 0.830 |
| OAS1 | DUSP6 | 0.830 |
| CTSL | IFIT5 | 0.830 |
| LGALS1 | CCR1 | 0.830 |
| HELZ2 | MX1 | 0.830 |
| IFIT2 | CCR1 | 0.830 |
| TNFSF10 | TSTD1 | 0.830 |
| HLA-F | IFI44 | 0.830 |
| MT2A | MX2 | 0.830 |
| BST2 | TSTD1 | 0.830 |
| MS4A6A | H4C8 | 0.830 |
| C1QC | SECTM1 | 0.830 |
| NKG7 | CD7 | 0.830 |
| LY6E | PARP14 | 0.830 |
| CD68 | RTP4 | 0.830 |
| CD68 | EIF2AK2 | 0.830 |
| IFIT3 | STAT2 | 0.830 |
| CXCL10 | IFI44 | 0.830 |
| NKG7 | DUSP6 | 0.830 |
| IFITM1 | GBP1 | 0.830 |
| IFIT3 | IFIT5 | 0.830 |
| IFIT2 | GBP3 | 0.830 |
| MT2A | IFI44L | 0.830 |
| TMSB10 | MT2A | 0.830 |
| HLA-A | IFIT5 | 0.830 |
| HLA-F | CD68 | 0.830 |
| SAMD9L | RSAD2 | 0.830 |
| CCR1 | RTP4 | 0.830 |
| TMSB10 | MX1 | 0.830 |
| IL2RG | IFITM3 | 0.830 |
| CTSL | TOR1B | 0.830 |
| LY6E | SOCS1 | 0.830 |
| OAS1 | SAMD9L | 0.829 |
| TIMP1 | KLHDC7B | 0.829 |
| CTSL | HLA-A | 0.829 |
| RSAD2 | TRIM22 | 0.829 |
| C1QC | TAP1 | 0.829 |
| TMEM199 | CXCL10 | 0.829 |
| STXBP2 | PARP14 | 0.829 |
| STXBP2 | EIF2AK2 | 0.829 |
| IL2RG | MAFB | 0.829 |
| IFIT3 | SAMD9L | 0.829 |
| IFIT5 | RSAD2 | 0.829 |
| FCER1G | PNMA1 | 0.829 |
| C1QC | TOR1B | 0.829 |
| C1QC | BST2 | 0.829 |
| TIMP1 | TYMP | 0.829 |
| CTSL | HELZ2 | 0.829 |
| SAMD9 | KLHDC7B | 0.829 |
| OAS3 | KLF6 | 0.829 |
| CD68 | MX2 | 0.829 |
| IFITM2 | GSTA1 | 0.829 |
| TNFSF10 | INPP5E | 0.829 |
| C1QC | SAMD9 | 0.829 |
| C1QC | OAS2 | 0.829 |
| H4C8 | IFIT3 | 0.829 |
| HLA-A | FCER1G | 0.829 |
| HLA-A | CXCL10 | 0.829 |
| CD7 | CXCL10 | 0.829 |
| IFI6 | BST2 | 0.829 |
| HLA-F | MT2A | 0.829 |
| XAF1 | STAT2 | 0.829 |
| IFI44L | IFI44 | 0.829 |
| RSAD2 | KLHDC7B | 0.829 |
| CXCL10 | TPRG1L | 0.829 |
| TLNRD1 | IFIT5 | 0.829 |
| SLC16A3 | IFIT5 | 0.829 |
| CDKN1C | SHFL | 0.829 |
| OAS1 | MX2 | 0.829 |
| TMSB10 | HELZ2 | 0.829 |
| TMEM199 | IFI44 | 0.829 |
| IFIT1 | PARP9 | 0.829 |
| OAS2 | IFI44L | 0.829 |
| OAS2 | SECTM1 | 0.829 |
| SLC16A3 | CTSL | 0.829 |
| CDKN1C | TRIM22 | 0.829 |
| NKG7 | GBP3 | 0.829 |
| FCER1G | IFITM2 | 0.829 |
| TOR1B | OAS3 | 0.829 |
| TOR1B | CD68 | 0.829 |
| CD68 | ISG20 | 0.829 |
| OAS2 | IFI35 | 0.829 |
| MT2A | PPP1R3D | 0.829 |
| OAS1 | SOCS1 | 0.828 |
| OAS1 | TNFSF10 | 0.828 |
| XAF1 | IFI44 | 0.828 |
| NKG7 | IL2RG | 0.828 |
| C6orf47 | OAS2 | 0.828 |
| MX2 | CCDC190 | 0.828 |
| BST2 | IFI44L | 0.828 |
| SAMD9 | IFI35 | 0.828 |
| HLA-B | IFIT3 | 0.828 |
| TMSB10 | CASP1 | 0.828 |
| TIMP1 | SECTM1 | 0.828 |
| SERTAD1 | TAP1 | 0.828 |
| NADK | IFI44 | 0.828 |
| IFIT3 | SOCS1 | 0.828 |
| MX2 | IFI44 | 0.828 |
| PPP1R3D | IFI44 | 0.828 |
| TYMP | CCDC190 | 0.828 |
| C1QC | SLC16A3 | 0.828 |
| C1QA | HLA-E | 0.828 |
| HLA-A | CD7 | 0.828 |
| LGALS1 | ATF5 | 0.828 |
| IFIT3 | EIF2AK2 | 0.828 |
| CXCL10 | MX1 | 0.828 |
| LGALS1 | KLF6 | 0.828 |
| SIGLEC10 | MX1 | 0.828 |
| OAS3 | IFI44 | 0.828 |
| SAMD9L | IFI44L | 0.828 |
| TRIM22 | PNMA1 | 0.828 |
| NKG7 | ATF5 | 0.827 |
| C6orf47 | CXCL10 | 0.827 |
| SIGLEC10 | OAS3 | 0.827 |
| TNFSF10 | CXCL10 | 0.827 |
| APOBEC | CCDC190 | 0.827 |
| 3G | | |
| IF144L | SHFL | 0.827 |
| C1QA | CASP1 | 0.827 |
| TMEM199 | STAT2 | 0.827 |
| H4C8 | IFI44 | 0.827 |
| CTSL | CCR1 | 0.827 |
| CD7 | MX2 | 0.827 |
| HLA-F | MX1 | 0.827 |
| XAF1 | MX2 | 0.827 |
| MS4A6A | RIPK3 | 0.827 |
| C1QA | SECTM1 | 0.827 |
| TMSB10 | EIF2AK2 | 0.827 |
| TIMP1 | FCER1G | 0.827 |
| CTSL | IFI35 | 0.827 |
| SAMD9 | IFITM2 | 0.827 |
| RSAD2 | STAT2 | 0.827 |
| FCER1G | GSTA1 | 0.827 |
| H4C8 | BST2 | 0.827 |
| STXBP2 | HLA-A | 0.827 |
| TMSB10 | BST2 | 0.827 |
| TOR1B | IFI44 | 0.827 |
| TMUB2 | CXCL10 | 0.827 |
| IFIT5 | IFI35 | 0.827 |
| IFIT5 | BST2 | 0.827 |
| SECTM1 | CCDC190 | 0.827 |
| TMSB10 | ISG20 | 0.827 |
| MAFB | DUSP6 | 0.827 |
| FCER1G | TOR1B | 0.827 |
| IFITM2 | SECTM1 | 0.827 |
| BST2 | PPP1R3D | 0.827 |
| TMSB10 | SERTAD1 | 0.827 |
| TIMP1 | TMEM199 | 0.827 |
| IL2RG | CXCL10 | 0.827 |
| SERTAD1 | SHFL | 0.827 |
| TMEM199 | BST2 | 0.827 |
| FCER1G | ISG20 | 0.827 |
| IFITM2 | KLHDC7B | 0.827 |
| IFITM2 | GBP3 | 0.827 |
| IFIT3 | OAS2 | 0.827 |
| IFIT3 | CCR1 | 0.827 |
| IFI44 | TPRG1L | 0.827 |
| MX2 | PRDX5 | 0.827 |
| KLHDC7B | INPP5E | 0.827 |
| C1QA | TNFSF10 | 0.826 |
| TMSB10 | IFI44 | 0.826 |
| TOR1B | MX1 | 0.826 |
| IFIT3 | RTP4 | 0.826 |
| SAMD9L | BST2 | 0.826 |
| CXCL10 | GBP3 | 0.826 |
| CXCL10 | CASP1 | 0.826 |
| HLA-B | IFI44 | 0.826 |
| TMSB10 | NADK | 0.826 |
| IRF7 | ATF5 | 0.826 |
| OAS2 | MT2A | 0.826 |
| SAMD9 | TPRG1L | 0.826 |
| EIF2AK2 | PRDX5 | 0.826 |
| TAP1 | SHFL | 0.826 |
| C1QA | PARP14 | 0.826 |
| TLNRD1 | CD68 | 0.826 |
| CTSL | SAMD9L | 0.826 |
| CD7 | ISG20 | 0.826 |
| NADK | SAMD9L | 0.826 |
| XAF1 | EIF2AK2 | 0.826 |
| MS4A6A | GBP3 | 0.826 |
| HLA-B | CXCL10 | 0.826 |
| IFI6 | GBP1 | 0.826 |
| IFIT1 | APOBEC3G | 0.826 |
| ISG20 | PNMA1 | 0.826 |
| TLNRD1 | CDKN1C | 0.826 |
| TLNRD1 | TMUB2 | 0.826 |
| C6orf47 | TAP1 | 0.826 |
| IL2RG | LGALS1 | 0.826 |
| LY6E | ATF5 | 0.826 |
| RIPK3 | IFIT3 | 0.826 |
| IFIT5 | ISG20 | 0.826 |
| MT2A | EIF2AK2 | 0.826 |
| CD7 | CCDC190 | 0.826 |
| OAS2 | ALDH3A1 | 0.826 |
| LGALS1 | GBP3 | 0.826 |
| SAMD9L | SHFL | 0.826 |
| STXBP2 | CCDC190 | 0.826 |
| TRIM22 | CCDC190 | 0.826 |
| IRF7 | CASP1 | 0.825 |
| BST2 | TPRG1L | 0.825 |
| IFIT3 | ISG20 | 0.825 |
| IFI44L | ATF5 | 0.825 |
| H4C8 | TYMP | 0.825 |
| C6orf47 | MT2A | 0.825 |
| TMSB10 | HLA-E | 0.825 |
| IFITM2 | SOCS1 | 0.825 |
| TLNRD1 | TYMP | 0.825 |
| CDKN1C | TOR1B | 0.825 |
| HELZ2 | IFIT5 | 0.825 |
| IFIT3 | TNFSF10 | 0.825 |
| OAS1 | SAMD9 | 0.825 |
| CD7 | NADK | 0.825 |
| TYMP | FCER1G | 0.825 |
| IFITM2 | TMUB2 | 0.825 |
| IRF7 | RTP4 | 0.825 |
| CCR1 | KLHDC7B | 0.825 |
| CASP1 | PNMA1 | 0.825 |
| TLNRD1 | C1QC | 0.825 |
| H4C8 | SAMD9L | 0.825 |
| HLA-A | STAT2 | 0.825 |
| TMEM199 | MT2A | 0.825 |
| HLA-F | SAMD9L | 0.825 |
| IFIT5 | SHFL | 0.825 |
| CXCL10 | SHFL | 0.825 |
| SECTM1 | ALDH3A1 | 0.825 |
| HLA-E | TPRG1L | 0.825 |
| TLNRD1 | NADK | 0.825 |
| TAP1 | IFIT3 | 0.825 |
| HLA-F | CXCL10 | 0.825 |
| KLF6 | IFI35 | 0.825 |
| KLF6 | MT2A | 0.825 |
| SAMD9L | DUSP6 | 0.825 |
| IFI35 | IFI44 | 0.825 |
| IFIT2 | PARP14 | 0.825 |
| BST2 | SHFL | 0.825 |
| CCR1 | CXCL10 | 0.825 |
| CDKN1C | CCDC190 | 0.825 |
| C1QC | STXBP2 | 0.825 |
| C1QC | EIF2AK2 | 0.825 |
| TAP1 | MX1 | 0.825 |
| LGALS1 | RIPK3 | 0.825 |
| HLA-F | SHFL | 0.825 |
| SECTM1 | CXCL10 | 0.825 |
| IRF7 | ALDH3A1 | 0.825 |
| XAF1 | ALDH3A1 | 0.825 |
| C1QC | CASP1 | 0.824 |
| H4C8 | CCR1 | 0.824 |
| OAS3 | PARP14 | 0.824 |
| IRF7 | TRIM22 | 0.824 |
| BST2 | IFI44 | 0.824 |
| MT2A | MX1 | 0.824 |
| IFI44L | PARP9 | 0.824 |
| TIMP1 | CCDC190 | 0.824 |
| HLA-A | PRDX5 | 0.824 |
| C1QC | NADK | 0.824 |
| IL2RG | RTP4 | 0.824 |
| TMUB2 | STAT2 | 0.824 |
| OAS2 | TNFSF10 | 0.824 |
| DUSP6 | RTP4 | 0.824 |
| DUSP6 | CXCL10 | 0.824 |
| TLNRD1 | HELZ2 | 0.824 |
| TMUB2 | RTP4 | 0.824 |
| TAP1 | ALDH3A1 | 0.824 |
| C1QC | DUSP6 | 0.824 |
| HLA-B | TIMP1 | 0.824 |
| NKG7 | KLF6 | 0.824 |
| TMSB10 | CCR1 | 0.824 |
| FCER1G | PARP14 | 0.824 |
| IFI44L | GBP3 | 0.824 |
| HLA-B | OAS2 | 0.824 |
| MAFB | KLF6 | 0.824 |
| HELZ2 | PNMA1 | 0.824 |
| HLA-F | PRDX5 | 0.824 |
| TLNRD1 | CASP1 | 0.824 |
| STXBP2 | TIMP1 | 0.824 |
| HLA-B | SAMD9L | 0.824 |
| TAP1 | BST2 | 0.824 |
| OAS2 | IFIT5 | 0.824 |
| CCR1 | MX1 | 0.824 |
| IFIT5 | SAMD9L | 0.823 |
| BST2 | SECTM1 | 0.823 |
| H4C8 | PRDX5 | 0.823 |
| H4C8 | IFI35 | 0.823 |
| C6orf47 | SAMD9L | 0.823 |
| ISG20 | TPRG1L | 0.823 |
| CDKN1C | PARP14 | 0.823 |
| SERTAD1 | BST2 | 0.823 |
| HLA-A | TOR1B | 0.823 |
| HLA-F | OAS2 | 0.823 |
| IFI35 | TNFSF10 | 0.823 |
| IFI35 | ATF5 | 0.823 |
| SAMD9 | INPP5E | 0.823 |
| TLNRD1 | TOR1B | 0.823 |
| TIMP1 | SOCS1 | 0.823 |
| SAMD9 | IFIT3 | 0.823 |
| NADK | HELZ2 | 0.823 |
| HLA-F | HELZ2 | 0.823 |
| IFITM2 | INPP5E | 0.823 |
| C1QA | PARP9 | 0.823 |
| TMSB10 | STAT2 | 0.823 |
| IFITM3 | GBP1 | 0.823 |
| CD68 | HLA-E | 0.823 |
| OAS2 | BST2 | 0.823 |
| IFIT5 | ATF5 | 0.823 |
| MX1 | CASP1 | 0.823 |
| CD68 | CCDC190 | 0.823 |
| H4C8 | GSTA1 | 0.823 |
| SERTAD1 | IFIT5 | 0.823 |
| FCER1G | RIPK3 | 0.823 |
| IRF7 | HLA-E | 0.823 |
| OAS2 | CXCL10 | 0.823 |
| IFI44L | STAT2 | 0.823 |
| CXCL10 | TSTD1 | 0.823 |
| TLNRD1 | SAMD9 | 0.822 |
| C1QA | HLA-A | 0.822 |
| OAS1 | TRIM22 | 0.822 |
| FCER1G | HLA-F | 0.822 |
| OAS3 | SOCS1 | 0.822 |
| XAF1 | OAS2 | 0.822 |
| IFI44L | MX1 | 0.822 |
| NKG7 | PRDX5 | 0.822 |
| SAMD9 | BST2 | 0.822 |
| IRF7 | SOCS1 | 0.822 |
| HLA-E | CXCL10 | 0.822 |
| HLA-E | MX1 | 0.822 |
| OAS2 | TPRG1L | 0.822 |
| SIGLEC10 | RTP4 | 0.822 |
| CD68 | TMUB2 | 0.822 |
| ISG20 | MX1 | 0.822 |
| MX2 | PNMA1 | 0.822 |
| FCER1G | INPP5E | 0.822 |
| IRF7 | DUSP6 | 0.822 |
| TAP1 | CD7 | 0.822 |
| CD7 | EIF2AK2 | 0.822 |
| OAS3 | GBP3 | 0.822 |
| SAMD9L | CCR1 | 0.822 |
| SAMD9L | MX1 | 0.822 |
| SECTM1 | EIF2AK2 | 0.822 |
| SECTM1 | INPP5E | 0.822 |
| CDKN1C | HLA-F | 0.822 |
| OAS1 | ATF5 | 0.822 |
| LY6E | KLHDC7B | 0.822 |
| SIGLEC10 | MT2A | 0.822 |
| TOR1B | IRF7 | 0.822 |
| HELZ2 | SECTM1 | 0.822 |
| IFIT1 | GBP1 | 0.822 |
| SAMD9L | TPRG1L | 0.822 |
| CDKN1C | TMSB10 | 0.822 |
| HLA-A | IFITM2 | 0.822 |
| IFITM2 | ATF5 | 0.822 |
| CD68 | KLHDC7B | 0.822 |
| MX2 | SHFL | 0.822 |
| OAS1 | HLA-E | 0.821 |
| TIMP1 | CTSL | 0.821 |
| TMEM199 | IFIT5 | 0.821 |
| FCER1G | ATF5 | 0.821 |
| TOR1B | IFITM2 | 0.821 |
| RSAD2 | GBP3 | 0.821 |
| C1QC | PNMA1 | 0.821 |
| TYMP | PNMA1 | 0.821 |
| IFIT5 | MX1 | 0.821 |
| SAMD9 | SHFL | 0.821 |
| RIPK3 | IRF7 | 0.821 |
| HELZ2 | SAMD9L | 0.821 |
| RSAD2 | PARP9 | 0.821 |
| LGALS1 | ALDH3A1 | 0.821 |
| ISG20 | CXCL10 | 0.821 |
| SECTM1 | CCR1 | 0.821 |
| C1QC | CCR1 | 0.821 |
| SERTAD1 | IFITM2 | 0.821 |
| CTSL | TAP1 | 0.821 |
| SECTM1 | RTP4 | 0.821 |
| TIMP1 | GSTA1 | 0.821 |
| MS4A6A | DUSP6 | 0.821 |
| HLA-B | HELZ2 | 0.821 |
| TMSB10 | HLA-F | 0.821 |
| TOR1B | IFIT2 | 0.821 |
| RIPK3 | CXCL10 | 0.821 |
| IFI35 | EIF2AK2 | 0.821 |
| TYMP | TSTD1 | 0.821 |
| TLNRD1 | PNMA1 | 0.821 |
| H4C8 | PNMA1 | 0.821 |
| C6orf47 | GSTA1 | 0.821 |
| TOR1B | INPP5E | 0.821 |
| PARP14 | INPP5E | 0.821 |
| C1QC | HLA-F | 0.821 |
| IFITM1 | APOBEC3G | 0.821 |
| C6orf47 | BST2 | 0.821 |
| TMSB10 | CD7 | 0.821 |
| IFI44L | RTP4 | 0.821 |
| NADK | PRDX5 | 0.821 |
| CD7 | TYMP | 0.821 |
| ISG20 | ALDH3A1 | 0.821 |
| TLNRD1 | TSTD1 | 0.821 |
| SLC16A3 | ISG20 | 0.820 |
| TYMP | TMEM199 | 0.820 |
| HELZ2 | OAS2 | 0.820 |
| HELZ2 | ISG20 | 0.820 |
| OAS2 | SHFL | 0.820 |
| SOCS1 | RSAD2 | 0.820 |
| HELZ2 | TSTD1 | 0.820 |
| CDKN1C | TNFSF10 | 0.820 |
| CXCL10 | PARP9 | 0.820 |
| IFI44 | ALDH3A1 | 0.820 |
| CD68 | CASP1 | 0.820 |
| CXCL10 | TRIM22 | 0.820 |
| C1QA | TYMP | 0.820 |
| HLA-B | TMSB10 | 0.820 |
| SERTAD1 | CTSL | 0.820 |
| IFIT3 | KLHDC7B | 0.820 |
| MAFB | ALDH3A1 | 0.820 |
| SLC16A3 | TPRG1L | 0.820 |
| OAS1 | IL2RG | 0.820 |
| LGALS1 | GBP1 | 0.820 |
| TOR1B | CCDC190 | 0.820 |
| MS4A6A | GBP1 | 0.820 |
| C1QA | SERTAD1 | 0.820 |
| CTSL | NADK | 0.820 |
| TYMP | BST2 | 0.820 |
| RIPK3 | OAS2 | 0.820 |
| TIMP1 | TPRG1L | 0.820 |
| C1QA | CTSL | 0.820 |
| CTSL | CD7 | 0.820 |
| IRF7 | PPP1R3D | 0.820 |
| BST2 | CXCL10 | 0.820 |
| TLNRD1 | EIF2AK2 | 0.820 |
| H4C8 | TIMP1 | 0.820 |
| C6orf47 | HELZ2 | 0.820 |
| CD68 | TRIM22 | 0.820 |
| KLF6 | RSAD2 | 0.820 |
| XAF1 | GBP3 | 0.820 |
| OAS2 | IFI44 | 0.820 |
| OAS2 | EIF2AK2 | 0.820 |
| TNFSF10 | IFI44 | 0.820 |
| BST2 | ALDH3A1 | 0.820 |
| BST2 | CCR1 | 0.819 |
| MT2A | ALDH3A1 | 0.819 |
| TLNRD1 | STAT2 | 0.819 |
| CDKN1C | MT2A | 0.819 |
| TIMP1 | CASP1 | 0.819 |
| SAMD9 | HELZ2 | 0.819 |
| MS4A6A | PPP1R3D | 0.819 |
| HLA-B | FCER1G | 0.819 |
| TAP1 | IFIT5 | 0.819 |
| NADK | BST2 | 0.819 |
| HELZ2 | IFI44 | 0.819 |
| C6orf47 | IFIT5 | 0.819 |
| SERTAD1 | HELZ2 | 0.819 |
| HELZ2 | BST2 | 0.819 |
| HELZ2 | SHFL | 0.819 |
| SOCS1 | INPP5E | 0.819 |
| NKG7 | RIPK3 | 0.819 |
| HLA-A | SAMD9 | 0.819 |
| SIGLEC10 | BST2 | 0.819 |
| IRF7 | GBP3 | 0.819 |
| KLF6 | CXCL10 | 0.819 |
| IFIT3 | MX2 | 0.819 |
| SAMD9L | IFI44 | 0.819 |
| OAS1 | RTP4 | 0.819 |
| TMSB10 | SHFL | 0.819 |
| TIMP1 | NADK | 0.819 |
| TAP1 | HELZ2 | 0.819 |
| XAF1 | PARP14 | 0.819 |
| MT2A | IFI44 | 0.819 |
| MX1 | SHFL | 0.819 |
| C1QC | TSTD1 | 0.819 |
| TIMP1 | PRDX5 | 0.819 |
| SAMD9L | KLHDC7B | 0.819 |
| CXCL10 | RTP4 | 0.819 |
| STXBP2 | TRIM22 | 0.818 |
| HLA-F | EIF2AK2 | 0.818 |
| IFIT5 | CCR1 | 0.818 |
| SAMD9L | MX2 | 0.818 |
| MT2A | TRIM22 | 0.818 |
| FCER1G | ALDH3A1 | 0.818 |
| NADK | TPRG1L | 0.818 |
| SHFL | TPRG1L | 0.818 |
| NADK | TSTD1 | 0.818 |
| IL2RG | IFIT2 | 0.818 |
| RIPK3 | MT2A | 0.818 |
| RIPK3 | IFI44 | 0.818 |
| HLA-F | STAT2 | 0.818 |
| SAMD9L | ISG20 | 0.818 |
| STAT2 | CXCL10 | 0.818 |
| SHFL | ALDH3A1 | 0.818 |
| HLA-F | TPRG1L | 0.818 |
| CTSL | TSTD1 | 0.818 |
| OAS1 | TOR1B | 0.818 |
| C1QA | SLC16A3 | 0.818 |
| FCER1G | TMUB2 | 0.818 |
| BST2 | MX1 | 0.818 |
| SAMD9L | ALDH3A1 | 0.818 |
| H4C8 | HLA-A | 0.818 |
| IFITM3 | SIGLEC10 | 0.818 |
| TLNRD1 | STXBP2 | 0.818 |
| STXBP2 | CCR1 | 0.818 |
| IFITM2 | PARP14 | 0.818 |
| RSAD2 | RTP4 | 0.818 |
| MT2A | TPRG1L | 0.818 |
| SAMD9 | SAMD9L | 0.818 |
| SOCS1 | BST2 | 0.818 |
| KLHDC7B | CASP1 | 0.818 |
| RTP4 | CASP1 | 0.818 |
| STAT2 | TSTD1 | 0.818 |
| HLA-F | GSTA1 | 0.818 |
| C1QC | TRIM22 | 0.818 |
| C1QA | DUSP6 | 0.818 |
| C6orf47 | ISG20 | 0.818 |
| TIMP1 | GBP1 | 0.818 |
| IFIT5 | IFI44 | 0.818 |
| HELZ2 | TPRG1L | 0.818 |
| MS4A6A | IL2RG | 0.817 |
| OAS1 | RIPK3 | 0.817 |
| TYMP | IFITM2 | 0.817 |
| TYMP | IFIT5 | 0.817 |
| KLF6 | OAS2 | 0.817 |
| IFIT5 | STAT2 | 0.817 |
| TLNRD1 | MX2 | 0.817 |
| CDKN1C | RTP4 | 0.817 |
| IL2RG | MX1 | 0.817 |
| CTSL | TYMP | 0.817 |
| TYMP | HELZ2 | 0.817 |
| SIGLEC10 | IFIT2 | 0.817 |
| HLA-B | SHFL | 0.817 |
| SAMD9 | IFI44 | 0.817 |
| NADK | IFIT5 | 0.817 |
| IFI35 | SHFL | 0.817 |
| IFIT2 | APOBEC3G | 0.817 |
| BST2 | CASP1 | 0.817 |
| PARP14 | PRDX5 | 0.817 |
| C1QA | NADK | 0.817 |
| H4C8 | TAP1 | 0.817 |
| CDKN1C | GBP1 | 0.817 |
| NKG7 | SIGLEC10 | 0.817 |
| SAMD9 | CCR1 | 0.817 |
| CD68 | CCR1 | 0.817 |
| IFIT3 | PARP14 | 0.817 |
| IFIT3 | GBP3 | 0.817 |
| BST2 | RTP4 | 0.817 |
| IFI44L | PARP14 | 0.817 |
| STXBP2 | KLHDC7B | 0.817 |
| NKG7 | GBP1 | 0.817 |
| IFITM3 | APOBEC3G | 0.817 |
| LY6E | RTP4 | 0.817 |
| CCR1 | STAT2 | 0.817 |
| CXCL10 | KLHDC7B | 0.817 |
| ATF5 | CCDC190 | 0.817 |
| TAP1 | SAMD9L | 0.817 |
| NADK | CD68 | 0.817 |
| NADK | STAT2 | 0.817 |
| FCER1G | MX2 | 0.817 |
| KLF6 | XAF1 | 0.817 |
| BST2 | MT2A | 0.817 |
| C1QC | ALDH3A1 | 0.817 |
| OAS1 | PARP14 | 0.817 |
| IL2RG | SAMD9L | 0.817 |
| IL2RG | KLHDC7B | 0.817 |
| RIPK3 | BST2 | 0.817 |
| IFIT5 | KLHDC7B | 0.817 |
| C6orf47 | CD68 | 0.816 |
| SERTAD1 | TNFSF10 | 0.816 |
| CTSL | CASP1 | 0.816 |
| IFI35 | PARP9 | 0.816 |
| IFIT2 | TRIM22 | 0.816 |
| SECTM1 | STAT2 | 0.816 |
| PPP1R3D | RTP4 | 0.816 |
| CXCL10 | PARP14 | 0.816 |
| TYMP | GSTA1 | 0.816 |
| HELZ2 | TNFSF10 | 0.816 |
| IFI35 | STAT2 | 0.816 |
| RTP4 | ALDH3A1 | 0.816 |
| TMEM199 | CD68 | 0.816 |
| FCER1G | TRIM22 | 0.816 |
| TOR1B | BST2 | 0.816 |
| IFI44 | EIF2AK2 | 0.816 |
| TOR1B | PRDX5 | 0.816 |
| CTSL | INPP5E | 0.816 |
| SAMD9 | MX1 | 0.816 |
| C1QA | RTP4 | 0.816 |
| C6orf47 | TIMP1 | 0.816 |
| TIMP1 | MX2 | 0.816 |
| TYMP | STAT2 | 0.816 |
| OAS2 | MX1 | 0.816 |
| OAS2 | PARP9 | 0.816 |
| SAMD9 | OAS2 | 0.816 |
| SIGLEC10 | IFIT3 | 0.816 |
| SAMD9L | CASP1 | 0.816 |
| BST2 | ISG20 | 0.816 |
| RSAD2 | PARP14 | 0.816 |
| MX1 | EIF2AK2 | 0.816 |
| C1QC | TMUB2 | 0.816 |
| CDKN1C | DUSP6 | 0.816 |
| IFITM2 | TRIM22 | 0.816 |
| IFIT2 | PPP1R3D | 0.816 |
| TMSB10 | TMEM199 | 0.815 |
| SERTAD1 | STAT2 | 0.815 |
| CTSL | STAT2 | 0.815 |
| CTSL | KLHDC7B | 0.815 |
| RIPK3 | MX1 | 0.815 |
| HLA-F | BST2 | 0.815 |
| HLA-F | CCDC190 | 0.815 |
| OAS1 | SIGLEC10 | 0.815 |
| HLA-A | ISG20 | 0.815 |
| BST2 | TNFSF10 | 0.815 |
| CASP1 | TSTD1 | 0.815 |
| MX2 | GSTA1 | 0.815 |
| TMSB10 | TRIM22 | 0.815 |
| IL2RG | BST2 | 0.815 |
| HLA-E | SHFL | 0.815 |
| C6orf47 | CCDC190 | 0.815 |
| TMEM199 | SECTM1 | 0.815 |
| OAS2 | ISG20 | 0.815 |
| MT2A | KLHDC7B | 0.815 |
| MX1 | ATF5 | 0.815 |
| TLNRD1 | IL2RG | 0.815 |
| CD68 | INPP5E | 0.815 |
| TLNRD1 | CD7 | 0.815 |
| SLC16A3 | CD68 | 0.815 |
| CDKN1C | CD68 | 0.815 |
| C6orf47 | MX2 | 0.815 |
| HLA-A | RTP4 | 0.815 |
| CD7 | CASP1 | 0.815 |
| IRF7 | PARP14 | 0.815 |
| CXCL10 | ATF5 | 0.815 |
| C1QC | IL2RG | 0.815 |
| C1QC | STAT2 | 0.815 |
| IFITM1 | PARP9 | 0.815 |
| MAFB | GBP1 | 0.815 |
| TAP1 | ISG20 | 0.815 |
| HELZ2 | MX2 | 0.815 |
| IFIT3 | TRIM22 | 0.815 |
| SOCS1 | CXCL10 | 0.815 |
| IFI44L | GBP1 | 0.815 |
| CCR1 | GSTA1 | 0.815 |
| TMUB2 | TNFSF10 | 0.814 |
| ISG20 | STAT2 | 0.814 |
| ISG20 | IFI44 | 0.814 |
| MX2 | MX1 | 0.814 |
| GBP3 | PNMA1 | 0.814 |
| HLA-B | IFITM2 | 0.814 |
| TOR1B | SHFL | 0.814 |
| CD68 | SOCS1 | 0.814 |
| TIMP1 | ALDH3A1 | 0.814 |
| SLC16A3 | TAP1 | 0.814 |
| SLC16A3 | SAMD9 | 0.814 |
| MX2 | KLHDC7B | 0.814 |
| TMEM199 | TSTD1 | 0.814 |
| TAP1 | EIF2AK2 | 0.814 |
| SIGLEC10 | IRF7 | 0.814 |
| SLC16A3 | KLHDC7B | 0.814 |
| SERTAD1 | CD7 | 0.814 |
| CTSL | TNFSF10 | 0.814 |
| LY6E | GBP1 | 0.814 |
| IFIT3 | CASP1 | 0.814 |
| MX2 | RTP4 | 0.814 |
| CDKN1C | HLA-A | 0.814 |
| SAMD9 | IFIT5 | 0.814 |
| TYMP | TMUB2 | 0.814 |
| OAS2 | SAMD9L | 0.814 |
| OAS2 | TRIM22 | 0.814 |
| IFI44 | RTP4 | 0.814 |
| CASP1 | CCDC190 | 0.814 |
| CASP1 | GSTA1 | 0.814 |
| C1QC | PARP14 | 0.814 |
| H4C8 | ISG20 | 0.814 |
| HLA-A | TAP1 | 0.814 |
| TYMP | ATF5 | 0.814 |
| TOR1B | PPP1R3D | 0.814 |
| HELZ2 | CCR1 | 0.814 |
| OAS2 | KLHDC7B | 0.814 |
| SAMD9L | HLA-E | 0.814 |
| IFI35 | TRIM22 | 0.814 |
| IFIT2 | CASP1 | 0.814 |
| TOR1B | GSTA1 | 0.814 |
| MAFB | SIGLEC10 | 0.813 |
| SAMD9 | ISG20 | 0.813 |
| TOR1B | IFIT3 | 0.813 |
| IFI44 | KLHDC7B | 0.813 |
| SLC16A3 | CDKN1C | 0.813 |
| MX1 | IFI44 | 0.813 |
| HLA-A | ALDH3A1 | 0.813 |
| CD68 | PARP14 | 0.813 |
| NADK | CCDC190 | 0.813 |
| TRIM22 | TPRG1L | 0.813 |
| MX2 | TSTD1 | 0.813 |
| PARP14 | PNMA1 | 0.813 |
| STXBP2 | GSTA1 | 0.813 |
| TIMP1 | CD68 | 0.813 |
| IFI6 | APOBEC3G | 0.813 |
| MT2A | PARP9 | 0.813 |
| MX2 | STAT2 | 0.813 |
| TNFSF10 | MX1 | 0.813 |
| IL2RG | IRF7 | 0.813 |
| CTSL | CD68 | 0.813 |
| SAMD9 | TYMP | 0.813 |
| IFI35 | KLHDC7B | 0.813 |
| MT2A | SHFL | 0.813 |
| HLA-A | GSTA1 | 0.813 |
| TMSB10 | CTSL | 0.813 |
| HLA-A | TMEM199 | 0.813 |
| CD7 | BST2 | 0.813 |
| C1QA | INPP5E | 0.813 |
| C1QA | TMUB2 | 0.812 |
| MAFB | RIPK3 | 0.812 |
| FCER1G | APOBEC3G | 0.812 |
| SOCS1 | MX1 | 0.812 |
| IFI44 | SHFL | 0.812 |
| C1QA | PNMA1 | 0.812 |
| TIMP1 | TMUB2 | 0.812 |
| TYMP | TNFSF10 | 0.812 |
| FCER1G | GBP1 | 0.812 |
| MX2 | SECTM1 | 0.812 |
| KLHDC7B | EIF2AK2 | 0.812 |
| HLA-A | CCDC190 | 0.812 |
| NADK | GSTA1 | 0.812 |
| C6orf47 | TYMP | 0.812 |
| HELZ2 | EIF2AK2 | 0.812 |
| C1QA | TSTD1 | 0.812 |
| OAS3 | PARP9 | 0.812 |
| TMSB10 | DUSP6 | 0.812 |
| C6orf47 | SAMD9 | 0.812 |
| NADK | RTP4 | 0.812 |
| SAMD9L | ATF5 | 0.812 |
| FCER1G | CCR1 | 0.812 |
| BST2 | STAT2 | 0.812 |
| IFITM2 | ALDH3A1 | 0.812 |
| C1QC | SERTAD1 | 0.812 |
| C1QC | SOCS1 | 0.812 |
| HLA-B | EIF2AK2 | 0.812 |
| IFITM2 | RIPK3 | 0.812 |
| IFITM2 | ISG20 | 0.812 |
| OAS3 | RTP4 | 0.812 |
| IFIT5 | TNFSF10 | 0.812 |
| IFIT5 | EIF2AK2 | 0.812 |
| SOCS1 | EIF2AK2 | 0.812 |
| BST2 | MX2 | 0.812 |
| ISG20 | PPP1R3D | 0.812 |
| HELZ2 | ALDH3A1 | 0.812 |
| HLA-F | PNMA1 | 0.812 |
| TIMP1 | PARP9 | 0.811 |
| CTSL | TMUB2 | 0.811 |
| TAP1 | TMUB2 | 0.811 |
| DUSP6 | BST2 | 0.811 |
| CASP1 | SHFL | 0.811 |
| CDKN1C | PNMA1 | 0.811 |
| XAF1 | PARP9 | 0.811 |
| EIF2AK2 | TPRG1L | 0.811 |
| CD7 | STAT2 | 0.811 |
| IFITM3 | PARP9 | 0.811 |
| IFIT2 | HLA-E | 0.811 |
| TAP1 | TYMP | 0.811 |
| OAS2 | MX2 | 0.811 |
| SOCS1 | IFI44 | 0.811 |
| TOR1B | TSTD1 | 0.811 |
| CDKN1C | SECTM1 | 0.811 |
| IL2RG | CTSL | 0.811 |
| IFI44 | ATF5 | 0.811 |
| H4C8 | MT2A | 0.811 |
| HLA-B | BST2 | 0.811 |
| HLA-A | TMUB2 | 0.811 |
| HELZ2 | STAT2 | 0.811 |
| XAF1 | RTP4 | 0.811 |
| CCR1 | EIF2AK2 | 0.811 |
| C1QC | PARP9 | 0.811 |
| STXBP2 | CTSL | 0.811 |
| RIPK3 | SAMD9L | 0.811 |
| HELZ2 | KLHDC7B | 0.811 |
| ATF5 | EIF2AK2 | 0.811 |
| SLC16A3 | PRDX5 | 0.811 |
| SAMD9 | STAT2 | 0.810 |
| HLA-F | IFIT5 | 0.810 |
| ISG20 | SECTM1 | 0.810 |
| H4C8 | CCDC190 | 0.810 |
| C1QC | INPP5E | 0.810 |
| SERTAD1 | HLA-F | 0.810 |
| NADK | TNFSF10 | 0.810 |
| TMEM199 | ISG20 | 0.810 |
| TOR1B | SAMD9L | 0.810 |
| CD7 | RTP4 | 0.810 |
| LY6E | GBP3 | 0.810 |
| IFIT5 | SOCS1 | 0.810 |
| TAP1 | ATF5 | 0.810 |
| IFI35 | PARP14 | 0.810 |
| STAT2 | MX1 | 0.810 |
| MS4A6A | PARP9 | 0.810 |
| C6orf47 | STAT2 | 0.810 |
| TMEM199 | CCR1 | 0.810 |
| TMEM199 | EIF2AK2 | 0.810 |
| TMEM199 | SHFL | 0.810 |
| MS4A6A | APOBEC3G | 0.810 |
| TAP1 | STAT2 | 0.810 |
| TYMP | SECTM1 | 0.810 |
| TOR1B | TMUB2 | 0.810 |
| TMUB2 | EIF2AK2 | 0.810 |
| RSAD2 | GBP1 | 0.810 |
| HLA-A | PNMA1 | 0.810 |
| OAS2 | ATF5 | 0.809 |
| STAT2 | IFI44 | 0.809 |
| CCR1 | TSTD1 | 0.809 |
| MS4A6A | KLF6 | 0.809 |
| C1QC | GBP1 | 0.809 |
| C1QA | IL2RG | 0.809 |
| H4C8 | IFIT5 | 0.809 |
| OAS1 | KLF6 | 0.809 |
| HLA-A | SECTM1 | 0.809 |
| IFIT3 | PPP1R3D | 0.809 |
| MT2A | TNFSF10 | 0.809 |
| CCR1 | INPP5E | 0.809 |
| TMSB10 | TNFSF10 | 0.809 |
| DUSP6 | SHFL | 0.809 |
| TAP1 | SECTM1 | 0.809 |
| STXBP2 | CD68 | 0.809 |
| NADK | FCER1G | 0.809 |
| BST2 | PARP14 | 0.809 |
| GBP1 | GSTA1 | 0.809 |
| C1QC | RTP4 | 0.809 |
| SLC16A3 | PARP14 | 0.809 |
| HLA-A | BST2 | 0.809 |
| OAS2 | RTP4 | 0.809 |
| IFI35 | GBP1 | 0.809 |
| HLA-A | ATF5 | 0.809 |
| TYMP | ISG20 | 0.809 |
| RIPK3 | IFI35 | 0.809 |
| IFIT5 | MX2 | 0.809 |
| MT2A | RTP4 | 0.809 |
| CD7 | ALDH3A1 | 0.809 |
| HLA-B | TPRG1L | 0.809 |
| C1QC | HLA-B | 0.808 |
| C1QC | HLA-E | 0.808 |
| C6orf47 | SHFL | 0.808 |
| TMSB10 | PPP1R3D | 0.808 |
| KLF6 | IFI44 | 0.808 |
| SOCS1 | MX2 | 0.808 |
| HLA-B | IFIT5 | 0.808 |
| IFITM1 | SIGLEC10 | 0.808 |
| IL2RG | IFIT3 | 0.808 |
| CTSL | HLA-F | 0.808 |
| TMUB2 | KLHDC7B | 0.808 |
| OAS2 | STAT2 | 0.808 |
| SAMD9L | STAT2 | 0.808 |
| HLA-F | ALDH3A1 | 0.808 |
| C1QA | GBP3 | 0.808 |
| H4C8 | SAMD9 | 0.808 |
| STXBP2 | CD7 | 0.808 |
| TAP1 | KLHDC7B | 0.808 |
| MX2 | TNFSF10 | 0.808 |
| TMSB10 | IL2RG | 0.808 |
| SAMD9L | TNFSF10 | 0.808 |
| TIMP1 | INPP5E | 0.808 |
| C6orf47 | CCR1 | 0.808 |
| SOCS1 | CCR1 | 0.808 |
| HLA-B | CD68 | 0.808 |
| MT2A | PARP14 | 0.808 |
| TNFSF10 | ALDH3A1 | 0.808 |
| HLA-B | PRDX5 | 0.808 |
| TAP1 | CCR1 | 0.808 |
| SAMD9 | SOCS1 | 0.808 |
| IFITM2 | HLA-F | 0.808 |
| IRF7 | KLF6 | 0.808 |
| TIMP1 | TSTD1 | 0.808 |
| PARP14 | TSTD1 | 0.808 |
| TLNRD1 | HLA-E | 0.808 |
| LGALS1 | SIGLEC10 | 0.808 |
| SAMD9L | EIF2AK2 | 0.808 |
| MT2A | STAT2 | 0.808 |
| PARP9 | PNMA1 | 0.808 |
| H4C8 | HELZ2 | 0.807 |
| CTSL | PARP9 | 0.807 |
| CTSL | RTP4 | 0.807 |
| HLA-A | SOCS1 | 0.807 |
| ISG20 | TNFSF10 | 0.807 |
| TNFSF10 | SECTM1 | 0.807 |
| IFI44 | TRIM22 | 0.807 |
| EIF2AK2 | SHFL | 0.807 |
| DUSP6 | TPRG1L | 0.807 |
| MX2 | GBP3 | 0.807 |
| CCR1 | PRDX5 | 0.807 |
| SLC16A3 | FCER1G | 0.807 |
| H4C8 | TMSB10 | 0.807 |
| CTSL | HLA-E | 0.807 |
| TOR1B | KLHDC7B | 0.807 |
| IL2RG | IFIT5 | 0.807 |
| HLA-A | PARP14 | 0.807 |
| HELZ2 | SOCS1 | 0.807 |
| C1QC | TPRG1L | 0.807 |
| CCR1 | TPRG1L | 0.807 |
| FCER1G | HLA-E | 0.807 |
| SAMD9L | RTP4 | 0.807 |
| STAT2 | CASP1 | 0.807 |
| HLA-B | PNMA1 | 0.807 |
| C1QC | TNFSF10 | 0.807 |
| H4C8 | SLC16A3 | 0.807 |
| H4C8 | CD68 | 0.807 |
| SLC16A3 | TIMP1 | 0.807 |
| STXBP2 | NADK | 0.807 |
| STXBP2 | GBP1 | 0.807 |
| MT2A | GBP1 | 0.807 |
| TNFSF10 | CCR1 | 0.807 |
| C1QA | ALDH3A1 | 0.807 |
| CTSL | DUSP6 | 0.807 |
| TYMP | EIF2AK2 | 0.807 |
| XAF1 | GBP1 | 0.807 |
| MX1 | TRIM22 | 0.807 |
| SOCS1 | PRDX5 | 0.807 |
| ATF5 | INPP5E | 0.807 |
| C1QA | CD7 | 0.806 |
| SERTAD1 | ISG20 | 0.806 |
| HELZ2 | ATF5 | 0.806 |
| CASP1 | TPRG1L | 0.806 |
| SLC16A3 | TRIM22 | 0.806 |
| STXBP2 | TOR1B | 0.806 |
| IFIT3 | DUSP6 | 0.806 |
| CCR1 | PNMA1 | 0.806 |
| TIMP1 | CCR1 | 0.806 |
| TAP1 | SAMD9 | 0.806 |
| TAP1 | MX2 | 0.806 |
| IFIT2 | GBP1 | 0.806 |
| IFIT2 | DUSP6 | 0.806 |
| H4C8 | DUSP6 | 0.806 |
| TYMP | PARP14 | 0.806 |
| SIGLEC10 | SAMD9L | 0.806 |
| HLA-E | BST2 | 0.806 |
| ISG20 | EIF2AK2 | 0.806 |
| SLC16A3 | SOCS1 | 0.806 |
| TOR1B | RTP4 | 0.806 |
| IFITM2 | PPP1R3D | 0.806 |
| CCDC190 | ALDH3A1 | 0.806 |
| C6orf47 | EIF2AK2 | 0.806 |
| SERTAD1 | RTP4 | 0.806 |
| TAP1 | PPP1R3D | 0.806 |
| OAS2 | SOCS1 | 0.806 |
| SAMD9 | TMEM199 | 0.806 |
| SAMD9 | ATF5 | 0.806 |
| IFITM2 | CCR1 | 0.806 |
| TNFSF10 | STAT2 | 0.806 |
| MX2 | TPRG1L | 0.806 |
| C1QA | ATF5 | 0.805 |
| CDKN1C | HLA-B | 0.805 |
| SAMD9L | SOCS1 | 0.805 |
| SECTM1 | ATF5 | 0.805 |
| CCR1 | GBP3 | 0.805 |
| TLNRD1 | TMSB10 | 0.805 |
| TLNRD1 | HLA-F | 0.805 |
| C1QA | CDKN1C | 0.805 |
| HELZ2 | RTP4 | 0.805 |
| SOCS1 | SHFL | 0.805 |
| CCR1 | CCDC190 | 0.805 |
| C1QA | HLA-B | 0.805 |
| CD7 | GSTA1 | 0.805 |
| SLC16A3 | GBP3 | 0.805 |
| MX1 | GBP3 | 0.805 |
| TYMP | ALDH3A1 | 0.805 |
| IFIT5 | ALDH3A1 | 0.805 |
| C1QA | TPRG1L | 0.805 |
| TIMP1 | PNMA1 | 0.805 |
| C6orf47 | TNFSF10 | 0.805 |
| CD7 | PARP14 | 0.805 |
| NADK | EIF2AK2 | 0.805 |
| SAMD9L | PPP1R3D | 0.805 |
| TLNRD1 | SERTAD1 | 0.805 |
| OAS3 | GBP1 | 0.805 |
| SLC16A3 | TOR1B | 0.805 |
| TIMP1 | SERTAD1 | 0.805 |
| HLA-A | TNFSF10 | 0.805 |
| KLF6 | MX1 | 0.805 |
| IFIT5 | HLA-E | 0.805 |
| MX2 | ATF5 | 0.805 |
| HLA-A | TSTD1 | 0.805 |
| HLA-A | INPP5E | 0.805 |
| STXBP2 | CASP1 | 0.804 |
| TMSB10 | RIPK3 | 0.804 |
| TAP1 | RTP4 | 0.804 |
| NADK | SOCS1 | 0.804 |
| TYMP | RTP4 | 0.804 |
| TLNRD1 | RTP4 | 0.804 |
| TIMP1 | HLA-E | 0.804 |
| SERTAD1 | FCER1G | 0.804 |
| SERTAD1 | KLHDC7B | 0.804 |
| CD7 | TOR1B | 0.804 |
| MX1 | RTP4 | 0.804 |
| CD7 | PNMA1 | 0.804 |
| TLNRD1 | TRIM22 | 0.804 |
| TLNRD1 | KLHDC7B | 0.804 |
| H4C8 | TOR1B | 0.804 |
| TMSB10 | KLF6 | 0.804 |
| IL2RG | SHFL | 0.804 |
| SAMD9 | TMUB2 | 0.804 |
| FCER1G | CASP1 | 0.804 |
| BST2 | ATF5 | 0.804 |
| BST2 | EIF2AK2 | 0.804 |
| ISG20 | CCR1 | 0.804 |
| TYMP | INPP5E | 0.804 |
| H4C8 | SOCS1 | 0.804 |
| NADK | SECTM1 | 0.804 |
| SIGLEC10 | SHFL | 0.804 |
| TMUB2 | SECTM1 | 0.804 |
| SOCS1 | TRIM22 | 0.804 |
| SERTAD1 | CCDC190 | 0.804 |
| HLA-F | INPP5E | 0.804 |
| TRIM22 | INPP5E | 0.804 |
| TLNRD1 | DUSP6 | 0.804 |
| SOCS1 | MT2A | 0.804 |
| ISG20 | PARP14 | 0.804 |
| C6orf47 | HLA-A | 0.804 |
| CD7 | TNFSF10 | 0.804 |
| HLA-F | ISG20 | 0.804 |
| IFI35 | SOCS1 | 0.804 |
| MX1 | PARP14 | 0.804 |
| IFI44 | PARP14 | 0.804 |
| C1QC | CDKN1C | 0.803 |
| TYMP | SOCS1 | 0.803 |
| RIPK3 | ISG20 | 0.803 |
| APOBEC3G | IFI44L | 0.803 |
| ISG20 | RTP4 | 0.803 |
| C1QC | PRDX5 | 0.803 |
| TAP1 | SOCS1 | 0.803 |
| SAMD9 | NADK | 0.803 |
| RIPK3 | IFIT5 | 0.803 |
| RIPK3 | RTP4 | 0.803 |
| TMUB2 | TSTD1 | 0.803 |
| OAS1 | GBP3 | 0.803 |
| TAP1 | TNFSF10 | 0.803 |
| DUSP6 | STAT2 | 0.803 |
| BST2 | GBP3 | 0.803 |
| GBP3 | PRDX5 | 0.803 |
| TOR1B | IFIT5 | 0.803 |
| HLA-A | TYMP | 0.803 |
| TYMP | CCR1 | 0.803 |
| SAMD9 | ALDH3A1 | 0.803 |
| C1QA | GBP1 | 0.803 |
| FCER1G | PARP9 | 0.803 |
| MX1 | KLHDC7B | 0.803 |
| NADK | PNMA1 | 0.803 |
| HLA-B | STAT2 | 0.803 |
| C6orf47 | TMSB10 | 0.803 |
| TNFSF10 | CASP1 | 0.803 |
| STXBP2 | ALDH3A1 | 0.803 |
| H4C8 | TSTD1 | 0.803 |
| CDKN1C | PRDX5 | 0.803 |
| CDKN1C | GSTA1 | 0.803 |
| HLA-B | TOR1B | 0.802 |
| IFIT5 | RTP4 | 0.802 |
| TMEM199 | CCDC190 | 0.802 |
| H4C8 | NADK | 0.802 |
| H4C8 | MX2 | 0.802 |
| TMSB10 | RTP4 | 0.802 |
| NADK | ISG20 | 0.802 |
| TMEM199 | RTP4 | 0.802 |
| TMUB2 | SOCS1 | 0.802 |
| GBP3 | TSTD1 | 0.802 |
| TOR1B | DUSP6 | 0.802 |
| TNFSF10 | EIF2AK2 | 0.802 |
| CCDC190 | TPRG1L | 0.802 |
| CTSL | TRIM22 | 0.802 |
| SAMD9 | TNFSF10 | 0.802 |
| RIPK3 | HELZ2 | 0.802 |
| C1QA | TMEM199 | 0.802 |
| STXBP2 | TSTD1 | 0.802 |
| SAMD9 | RTP4 | 0.802 |
| CD7 | HLA-F | 0.802 |
| HELZ2 | PARP14 | 0.802 |
| OAS2 | GBP3 | 0.802 |
| ISG20 | ATF5 | 0.802 |
| ISG20 | KLHDC7B | 0.802 |
| TRIM22 | SHFL | 0.802 |
| STAT2 | TPRG1L | 0.802 |
| OAS1 | GBP1 | 0.802 |
| TMEM199 | MX2 | 0.802 |
| IFI44 | GBP3 | 0.802 |
| C1QC | H4C8 | 0.801 |
| TOR1B | HLA-F | 0.801 |
| OAS2 | PARP14 | 0.801 |
| RTP4 | TPRG1L | 0.801 |
| SERTAD1 | TRIM22 | 0.801 |
| FCER1G | PPP1R3D | 0.801 |
| HELZ2 | TRIM22 | 0.801 |
| TLNRD1 | SHFL | 0.801 |
| CDKN1C | KLF6 | 0.801 |
| SERTAD1 | SAMD9 | 0.801 |
| KLF6 | IFIT2 | 0.801 |
| KLF6 | BST2 | 0.801 |
| PARP9 | TPRG1L | 0.801 |
| HLA-F | SECTM1 | 0.801 |
| HELZ2 | CASP1 | 0.801 |
| IFIT3 | HLA-E | 0.801 |
| IFIT5 | PPP1R3D | 0.801 |
| DUSP6 | KLHDC7B | 0.801 |
| ISG20 | MX2 | 0.801 |
| STAT2 | ALDH3A1 | 0.801 |
| TRIM22 | TSTD1 | 0.801 |
| TLNRD1 | PARP9 | 0.801 |
| C1QA | KLF6 | 0.801 |
| LGALS1 | APOBEC3G | 0.801 |
| SOCS1 | ISG20 | 0.801 |
| HLA-B | GSTA1 | 0.801 |
| SLC16A3 | STXBP2 | 0.801 |
| SLC16A3 | ATF5 | 0.801 |
| SERTAD1 | PARP14 | 0.801 |
| TAP1 | RIPK3 | 0.801 |
| BST2 | KLHDC7B | 0.801 |
| MX2 | PARP14 | 0.801 |
| STAT2 | SHFL | 0.801 |
| PNMA1 | GSTA1 | 0.801 |
| CTSL | TPRG1L | 0.801 |
| STXBP2 | HLA-B | 0.800 |
| TMSB10 | SOCS1 | 0.800 |
| CTSL | PARP14 | 0.800 |
| SAMD9L | GBP3 | 0.800 |
| HLA-E | RTP4 | 0.800 |
| CXCL10 | GBP1 | 0.800 |
| H4C8 | KLHDC7B | 0.800 |
| SLC16A3 | TYMP | 0.800 |
| TMSB10 | PARP14 | 0.800 |
| HLA-F | TSTD1 | 0.800 |
| C1QA | C6orf47 | 0.800 |
| SLC16A3 | HLA-A | 0.800 |
| TOR1B | TNFSF10 | 0.800 |
| CTSL | ALDH3A1 | 0.800 |
| C1QA | SOCS1 | 0.800 |
| C1QC | CTSL | 0.800 |
| C1QC | CD7 | 0.800 |
| CCR1 | ATF5 | 0.800 |
| STAT2 | EIF2AK2 | 0.800 |
| TLNRD1 | SOCS1 | 0.800 |
| C1QC | SIGLEC10 | 0.800 |
| HLA-B | SAMD9 | 0.800 |
| SERTAD1 | EIF2AK2 | 0.800 |
| TAP1 | NADK | 0.800 |
| TYMP | PPP1R3D | 0.800 |
| TYMP | KLHDC7B | 0.800 |
| HLA-F | CCR1 | 0.800 |
| ALDH3A1 | TSTD1 | 0.800 |
| TNFSF10 | TPRG1L | 0.800 |
| SOCS1 | PNMA1 | 0.800 |
| SECTM1 | PNMA1 | 0.800 |
| SLC16A3 | C6orf47 | 0.799 |
| STXBP2 | TMEM199 | 0.799 |
| CDKN1C | CD7 | 0.799 |
| HLA-A | CCR1 | 0.799 |
| LY6E | APOBEC3G | 0.799 |
| FCER1G | DUSP6 | 0.799 |
| CD68 | ATF5 | 0.799 |
| SOCS1 | ALDH3A1 | 0.799 |
| TMEM199 | GSTA1 | 0.799 |
| HLA-A | CASP1 | 0.799 |
| IRF7 | GBP1 | 0.799 |
| KLF6 | SAMD9L | 0.799 |
| IFIT5 | CASP1 | 0.799 |
| SAMD9L | PARP14 | 0.799 |
| SOCS1 | CASP1 | 0.799 |
| IL2RG | PNMA1 | 0.799 |
| IFIT5 | PARP14 | 0.799 |
| STAT2 | ATF5 | 0.799 |
| C1QA | PRDX5 | 0.799 |
| STXBP2 | CDKN1C | 0.799 |
| SLC16A3 | IFITM2 | 0.799 |
| NKG7 | APOBEC3G | 0.799 |
| C6orf47 | SECTM1 | 0.799 |
| CD68 | PARP9 | 0.799 |
| SAMD9L | TRIM22 | 0.799 |
| SOCS1 | TSTD1 | 0.799 |
| HLA-B | CD7 | 0.799 |
| TIMP1 | DUSP6 | 0.799 |
| HLA-A | MX2 | 0.799 |
| H4C8 | CD7 | 0.799 |
| CDKN1C | SERTAD1 | 0.799 |
| HLA-B | RTP4 | 0.799 |
| OAS1 | PARP9 | 0.799 |
| TYMP | TOR1B | 0.799 |
| PARP9 | IFI44 | 0.799 |
| ALDH3A1 | PNMA1 | 0.798 |
| TPRG1L | GSTA1 | 0.798 |
| CDKN1C | ALDH3A1 | 0.798 |
| SECTM1 | TSTD1 | 0.798 |
| C6orf47 | RTP4 | 0.798 |
| TOR1B | HELZ2 | 0.798 |
| DUSP6 | TNFSF10 | 0.798 |
| GBP1 | CCDC190 | 0.798 |
| KLHDC7B | PNMA1 | 0.798 |
| C1QC | GBP3 | 0.798 |
| NADK | ATF5 | 0.798 |
| TOR1B | ISG20 | 0.798 |
| HLA-F | TNFSF10 | 0.798 |
| HLA-F | RTP4 | 0.798 |
| IFITM2 | APOBEC3G | 0.798 |
| CCR1 | PARP14 | 0.798 |
| IFIT2 | PARP9 | 0.798 |
| IFI35 | RTP4 | 0.798 |
| SECTM1 | PARP14 | 0.798 |
| C1QA | KLHDC7B | 0.798 |
| NADK | PARP14 | 0.798 |
| TYMP | HLA-F | 0.798 |
| RIPK3 | SHFL | 0.798 |
| IFIT5 | GBP3 | 0.798 |
| CD7 | INPP5E | 0.798 |
| NADK | INPP5E | 0.798 |
| HLA-A | DUSP6 | 0.797 |
| GBP1 | IFI44 | 0.797 |
| C1QA | H4C8 | 0.797 |
| SLC16A3 | SECTM1 | 0.797 |
| SERTAD1 | GSTA1 | 0.797 |
| TAP1 | TOR1B | 0.797 |
| CD68 | PPP1R3D | 0.797 |
| DUSP6 | EIF2AK2 | 0.797 |
| APOBEC3G | CXCL10 | 0.797 |
| SECTM1 | TPRG1L | 0.797 |
| STXBP2 | ATF5 | 0.797 |
| TYMP | MX2 | 0.797 |
| HLA-B | CCDC190 | 0.797 |
| CD68 | TSTD1 | 0.797 |
| FCER1G | KLF6 | 0.797 |
| IFITM2 | GBP1 | 0.797 |
| STAT2 | KLHDC7B | 0.797 |
| KLHDC7B | SHFL | 0.797 |
| SLC16A3 | CCDC190 | 0.797 |
| TLNRD1 | PARP14 | 0.796 |
| CD7 | TRIM22 | 0.796 |
| TOR1B | ATF5 | 0.796 |
| OAS2 | GBP1 | 0.796 |
| SOCS1 | SECTM1 | 0.796 |
| ATF5 | CASP1 | 0.796 |
| TMSB10 | PARP9 | 0.796 |
| IFIT5 | DUSP6 | 0.796 |
| IFI35 | GBP3 | 0.796 |
| MT2A | ATF5 | 0.796 |
| SERTAD1 | TYMP | 0.796 |
| TOR1B | GBP3 | 0.796 |
| MX2 | CCR1 | 0.796 |
| SECTM1 | PRDX5 | 0.796 |
| H4C8 | INPP5E | 0.796 |
| CTSL | GBP1 | 0.796 |
| H4C8 | TPRG1L | 0.796 |
| IFITM2 | PARP9 | 0.796 |
| CDKN1C | INPP5E | 0.796 |
| SERTAD1 | MX2 | 0.796 |
| SAMD9 | MX2 | 0.796 |
| TOR1B | SOCS1 | 0.796 |
| KLHDC7B | ALDH3A1 | 0.796 |
| TLNRD1 | HLA-B | 0.796 |
| C1QA | RIPK3 | 0.796 |
| C6orf47 | NADK | 0.796 |
| TOR1B | STAT2 | 0.796 |
| IRF7 | PARP9 | 0.796 |
| MX2 | EIF2AK2 | 0.796 |
| PARP9 | CCDC190 | 0.796 |
| SAMD9 | EIF2AK2 | 0.795 |
| TMUB2 | ATF5 | 0.795 |
| KLHDC7B | PRDX5 | 0.795 |
| C6orf47 | TOR1B | 0.795 |
| SIGLEC10 | STAT2 | 0.795 |
| TLNRD1 | ALDH3A1 | 0.795 |
| CCR1 | ALDH3A1 | 0.795 |
| STXBP2 | PNMA1 | 0.795 |
| GBP1 | PRDX5 | 0.795 |
| CASP1 | INPP5E | 0.795 |
| CDKN1C | PARP9 | 0.795 |
| IL2RG | STAT2 | 0.795 |
| CD7 | SECTM1 | 0.795 |
| H4C8 | SERTAD1 | 0.795 |
| SAMD9 | HLA-F | 0.795 |
| TOR1B | SECTM1 | 0.795 |
| HLA-F | MX2 | 0.795 |
| CD68 | GBP1 | 0.795 |
| TMUB2 | GSTA1 | 0.795 |
| NADK | TYMP | 0.795 |
| TYMP | GBP1 | 0.795 |
| TMEM199 | TOR1B | 0.795 |
| TMUB2 | PARP14 | 0.795 |
| SOCS1 | DUSP6 | 0.795 |
| HLA-E | STAT2 | 0.795 |
| BST2 | TRIM22 | 0.795 |
| MT2A | GBP3 | 0.795 |
| SERTAD1 | PNMA1 | 0.795 |
| CDKN1C | GBP3 | 0.795 |
| TIMP1 | ATF5 | 0.795 |
| IL2RG | HELZ2 | 0.795 |
| MAFB | APOBEC3G | 0.795 |
| RTP4 | EIF2AK2 | 0.795 |
| ALDH3A1 | GSTA1 | 0.795 |
| TLNRD1 | TNFSF10 | 0.794 |
| HLA-A | KLHDC7B | 0.794 |
| HLA-F | TMUB2 | 0.794 |
| SECTM1 | KLHDC7B | 0.794 |
| SLC16A3 | HLA-F | 0.794 |
| H4C8 | CASP1 | 0.794 |
| C6orf47 | CASP1 | 0.794 |
| HLA-A | NADK | 0.794 |
| CD68 | GBP3 | 0.794 |
| SECTM1 | TRIM22 | 0.794 |
| TOR1B | EIF2AK2 | 0.794 |
| H4C8 | RTP4 | 0.794 |
| RIPK3 | STAT2 | 0.794 |
| IRF7 | APOBEC3G | 0.794 |
| KLF6 | IFIT3 | 0.794 |
| SLC16A3 | PNMA1 | 0.794 |
| SERTAD1 | CASP1 | 0.794 |
| KLF6 | SHFL | 0.794 |
| IFIT3 | GBP1 | 0.794 |
| SLC16A3 | TSTD1 | 0.794 |
| NADK | TOR1B | 0.793 |
| SECTM1 | GBP3 | 0.793 |
| C1QC | TMEM199 | 0.793 |
| CTSL | SHFL | 0.793 |
| IFITM2 | KLF6 | 0.793 |
| IFIT5 | TRIM22 | 0.793 |
| APOBEC3G | RSAD2 | 0.793 |
| ISG20 | TRIM22 | 0.793 |
| HLA-A | TRIM22 | 0.793 |
| HLA-B | ISG20 | 0.793 |
| IL2RG | CD68 | 0.793 |
| HELZ2 | HLA-E | 0.793 |
| SECTM1 | CASP1 | 0.793 |
| TAP1 | HLA-F | 0.793 |
| IL2RG | FCER1G | 0.793 |
| TMEM199 | TNFSF10 | 0.793 |
| TMEM199 | KLHDC7B | 0.793 |
| SERTAD1 | TPRG1L | 0.793 |
| CDKN1C | TMUB2 | 0.793 |
| HLA-B | TAP1 | 0.793 |
| SERTAD1 | SECTM1 | 0.793 |
| IFIT3 | APOBEC3G | 0.793 |
| GBP1 | ALDH3A1 | 0.793 |
| TNFSF10 | ATF5 | 0.793 |
| SLC16A3 | GSTA1 | 0.793 |
| SERTAD1 | TOR1B | 0.792 |
| RIPK3 | SECTM1 | 0.792 |
| RIPK3 | KLHDC7B | 0.792 |
| SOCS1 | STAT2 | 0.792 |
| CCR1 | TRIM22 | 0.792 |
| SERTAD1 | PRDX5 | 0.792 |
| C1QC | KLF6 | 0.792 |
| SIGLEC10 | IFIT5 | 0.792 |
| HLA-F | SOCS1 | 0.792 |
| CDKN1C | TSTD1 | 0.792 |
| H4C8 | HLA-F | 0.792 |
| H4C8 | EIF2AK2 | 0.792 |
| TIMP1 | RIPK3 | 0.792 |
| C6orf47 | HLA-F | 0.792 |
| SERTAD1 | HLA-A | 0.792 |
| HLA-F | ATF5 | 0.792 |
| HLA-E | ISG20 | 0.792 |
| GBP3 | CASP1 | 0.792 |
| HLA-E | EIF2AK2 | 0.792 |
| TOR1B | ALDH3A1 | 0.792 |
| IFIT5 | GBP1 | 0.792 |
| CD68 | DUSP6 | 0.792 |
| KLF6 | IFIT5 | 0.792 |
| H4C8 | IL2RG | 0.792 |
| TAP1 | GBP3 | 0.792 |
| ATF5 | SHFL | 0.792 |
| TRIM22 | KLHDC7B | 0.792 |
| ALDH3A1 | PRDX5 | 0.792 |
| C1QC | RIPK3 | 0.791 |
| TOR1B | CASP1 | 0.791 |
| CCDC190 | PNMA1 | 0.791 |
| IL2RG | CCDC190 | 0.791 |
| STXBP2 | APOBEC3G | 0.791 |
| IL2RG | TAP1 | 0.791 |
| SAMD9 | PARP14 | 0.791 |
| CD7 | TMEM199 | 0.791 |
| NADK | IFITM2 | 0.791 |
| KLF6 | PNMA1 | 0.791 |
| C1QA | SIGLEC10 | 0.791 |
| HLA-B | CCR1 | 0.791 |
| SERTAD1 | CCR1 | 0.791 |
| SIGLEC10 | TNFSF10 | 0.791 |
| SAMD9L | GBP1 | 0.791 |
| CCDC190 | GSTA1 | 0.791 |
| TYMP | CASP1 | 0.791 |
| C6orf47 | PRDX5 | 0.791 |
| DUSP6 | ISG20 | 0.791 |
| PARP14 | EIF2AK2 | 0.791 |
| NADK | ALDH3A1 | 0.791 |
| TLNRD1 | TMEM199 | 0.791 |
| NADK | TRIM22 | 0.791 |
| GBP1 | SHFL | 0.791 |
| NADK | TMEM199 | 0.790 |
| SIGLEC10 | HELZ2 | 0.790 |
| TMUB2 | MX2 | 0.790 |
| MX2 | ALDH3A1 | 0.790 |
| GBP1 | PNMA1 | 0.790 |
| H4C8 | SECTM1 | 0.790 |
| TIMP1 | KLF6 | 0.790 |
| TAP1 | TRIM22 | 0.790 |
| HLA-F | PARP14 | 0.790 |
| HELZ2 | PPP1R3D | 0.790 |
| MX1 | PARP9 | 0.790 |
| EIF2AK2 | CASP1 | 0.790 |
| PARP14 | ALDH3A1 | 0.790 |
| IFITM2 | CASP1 | 0.790 |
| SERTAD1 | TSTD1 | 0.790 |
| C1QC | PPP1R3D | 0.790 |
| STXBP2 | SERTAD1 | 0.790 |
| HLA-B | CTSL | 0.790 |
| C6orf47 | PARP14 | 0.790 |
| NADK | KLHDC7B | 0.790 |
| RIPK3 | CCR1 | 0.790 |
| TIMP1 | PPP1R3D | 0.790 |
| IL2RG | SAMD9 | 0.790 |
| TMEM199 | CASP1 | 0.790 |
| TYMP | TRIM22 | 0.790 |
| APOBEC3G | IFI44 | 0.790 |
| CD7 | KLHDC7B | 0.790 |
| TMUB2 | CCR1 | 0.790 |
| C1QC | KLHDC7B | 0.789 |
| OAS1 | APOBEC3G | 0.789 |
| TMEM199 | PARP14 | 0.789 |
| HELZ2 | DUSP6 | 0.789 |
| BST2 | GBP1 | 0.789 |
| ISG20 | GBP3 | 0.789 |
| CD68 | ALDH3A1 | 0.789 |
| TRIM22 | ALDH3A1 | 0.789 |
| RIPK3 | TNFSF10 | 0.789 |
| RTP4 | SHFL | 0.789 |
| IL2RG | TPRG1L | 0.789 |
| ATF5 | TSTD1 | 0.789 |
| HLA-E | PNMA1 | 0.789 |
| CASP1 | ALDH3A1 | 0.789 |
| CD68 | PRDX5 | 0.789 |
| ATF5 | TRIM22 | 0.789 |
| HLA-E | TNFSF10 | 0.789 |
| C1QA | PPP1R3D | 0.789 |
| KLHDC7B | RTP4 | 0.789 |
| STXBP2 | TPRG1L | 0.789 |
| HLA-E | PRDX5 | 0.789 |
| SERTAD1 | GBP3 | 0.789 |
| SAMD9 | TOR1B | 0.789 |
| RIPK3 | CD68 | 0.789 |
| PARP14 | SHFL | 0.789 |
| TLNRD1 | KLF6 | 0.789 |
| CDKN1C | KLHDC7B | 0.789 |
| HLA-B | SECTM1 | 0.789 |
| CD7 | GBP3 | 0.789 |
| ISG20 | CASP1 | 0.789 |
| TNFSF10 | PARP14 | 0.789 |
| TNFSF10 | SHFL | 0.789 |
| TRIM22 | RTP4 | 0.789 |
| KLF6 | TPRG1L | 0.789 |
| EIF2AK2 | ALDH3A1 | 0.788 |
| PARP14 | TPRG1L | 0.788 |
| CD7 | TSTD1 | 0.788 |
| DUSP6 | PNMA1 | 0.788 |
| SERTAD1 | SOCS1 | 0.788 |
| NADK | HLA-F | 0.788 |
| TMSB10 | SIGLEC10 | 0.788 |
| KLF6 | RTP4 | 0.788 |
| IFIT5 | APOBEC3G | 0.788 |
| C1QC | ATF5 | 0.788 |
| TAP1 | CASP1 | 0.788 |
| SAMD9 | PPP1R3D | 0.788 |
| CD68 | PNMA1 | 0.788 |
| STAT2 | RTP4 | 0.788 |
| H4C8 | ALDH3A1 | 0.788 |
| HLA-A | GBP1 | 0.788 |
| TOR1B | CCR1 | 0.788 |
| IFIT3 | PARP9 | 0.788 |
| HLA-E | KLHDC7B | 0.788 |
| GBP1 | EIF2AK2 | 0.788 |
| TMUB2 | TPRG1L | 0.788 |
| KLHDC7B | TSTD1 | 0.788 |
| PPP1R3D | PNMA1 | 0.788 |
| BST2 | PARP9 | 0.787 |
| MX2 | GBP1 | 0.787 |
| PPP1R3D | EIF2AK2 | 0.787 |
| ATF5 | ALDH3A1 | 0.787 |
| APOBEC3G | PNMA1 | 0.787 |
| C6orf47 | INPP5E | 0.787 |
| IL2RG | TYMP | 0.787 |
| TAP1 | PARP14 | 0.787 |
| APOBEC3G | CCR1 | 0.787 |
| HLA-B | TYMP | 0.787 |
| CTSL | SIGLEC10 | 0.787 |
| TNFSF10 | RTP4 | 0.787 |
| OAS2 | APOBEC3G | 0.787 |
| SERTAD1 | GBP1 | 0.787 |
| GBP1 | MX1 | 0.787 |
| HLA-B | ALDH3A1 | 0.787 |
| H4C8 | PARP9 | 0.787 |
| HLA-B | PARP14 | 0.787 |
| STAT2 | PARP14 | 0.787 |
| MX2 | INPP5E | 0.787 |
| HLA-A | PPP1R3D | 0.787 |
| SAMD9 | SIGLEC10 | 0.787 |
| C1QC | C1QA | 0.786 |
| IFITM2 | MX2 | 0.786 |
| PPP1R3D | PARP9 | 0.786 |
| RTP4 | GBP3 | 0.786 |
| PPP1R3D | TPRG1L | 0.786 |
| H4C8 | TRIM22 | 0.786 |
| IL2RG | EIF2AK2 | 0.786 |
| DUSP6 | GBP3 | 0.786 |
| IL2RG | TNFSF10 | 0.786 |
| CTSL | SOCS1 | 0.786 |
| SOCS1 | HLA-E | 0.786 |
| CCDC190 | PRDX5 | 0.786 |
| TMUB2 | CCDC190 | 0.786 |
| H4C8 | STXBP2 | 0.786 |
| CD7 | GBP1 | 0.786 |
| TMEM199 | TRIM22 | 0.786 |
| SERTAD1 | TMUB2 | 0.786 |
| IFITM2 | DUSP6 | 0.786 |
| TLNRD1 | TPRG1L | 0.786 |
| HLA-B | TSTD1 | 0.786 |
| IL2RG | IFITM2 | 0.786 |
| DUSP6 | GSTA1 | 0.786 |
| CDKN1C | HLA-E | 0.786 |
| CTSL | TMEM199 | 0.786 |
| TMEM199 | HLA-F | 0.786 |
| TOR1B | PARP14 | 0.786 |
| HELZ2 | GBP3 | 0.786 |
| H4C8 | PARP14 | 0.785 |
| IL2RG | SOCS1 | 0.785 |
| KLF6 | KLHDC7B | 0.785 |
| STXBP2 | INPP5E | 0.785 |
| IL2RG | CD7 | 0.785 |
| IL2RG | GBP3 | 0.785 |
| RTP4 | PARP14 | 0.785 |
| ALDH3A1 | INPP5E | 0.785 |
| H4C8 | CDKN1C | 0.785 |
| CDKN1C | SOCS1 | 0.785 |
| SERTAD1 | CD68 | 0.785 |
| TMUB2 | GBP3 | 0.785 |
| SLC16A3 | CASP1 | 0.785 |
| SAMD9 | GBP1 | 0.785 |
| APOBEC3G | PRDX5 | 0.785 |
| TIMP1 | IL2RG | 0.785 |
| TAP1 | GBP1 | 0.785 |
| SAMD9 | CASP1 | 0.785 |
| TMUB2 | TRIM22 | 0.785 |
| PARP9 | PRDX5 | 0.785 |
| PARP9 | GSTA1 | 0.785 |
| H4C8 | STAT2 | 0.785 |
| TMSB10 | KLHDC7B | 0.785 |
| TYMP | RIPK3 | 0.785 |
| TOR1B | HLA-E | 0.785 |
| XAF1 | APOBEC3G | 0.785 |
| MX2 | PPP1R3D | 0.785 |
| MS4A6A | SIGLEC10 | 0.784 |
| H4C8 | TMUB2 | 0.784 |
| IL2RG | TOR1B | 0.784 |
| SIGLEC10 | SOCS1 | 0.784 |
| HLA-E | ATF5 | 0.784 |
| RIPK3 | GSTA1 | 0.784 |
| SIGLEC10 | KLHDC7B | 0.784 |
| SIGLEC10 | PARP14 | 0.784 |
| CD68 | TPRG1L | 0.784 |
| GBP3 | INPP5E | 0.784 |
| TLNRD1 | RIPK3 | 0.784 |
| TLNRD1 | GBP1 | 0.784 |
| HLA-B | SOCS1 | 0.784 |
| CD7 | SOCS1 | 0.784 |
| TOR1B | MX2 | 0.784 |
| IFITM2 | HLA-E | 0.784 |
| SLC16A3 | GBP1 | 0.784 |
| HLA-B | KLHDC7B | 0.784 |
| SAMD9 | DUSP6 | 0.784 |
| HLA-E | CCDC190 | 0.784 |
| SLC16A3 | SERTAD1 | 0.783 |
| HLA-B | TMUB2 | 0.783 |
| HLA-A | HLA-F | 0.783 |
| TYMP | GBP3 | 0.783 |
| HLA-F | TRIM22 | 0.783 |
| CD68 | APOBEC3G | 0.783 |
| GBP3 | EIF2AK2 | 0.783 |
| C6orf47 | TRIM22 | 0.783 |
| TMEM199 | SOCS1 | 0.783 |
| GBP1 | INPP5E | 0.783 |
| OAS3 | APOBEC3G | 0.783 |
| GBP1 | TPRG1L | 0.783 |
| APOBEC3G | INPP5E | 0.783 |
| STAT2 | TRIM22 | 0.783 |
| TMSB10 | GBP1 | 0.783 |
| SIGLEC10 | TOR1B | 0.783 |
| TMUB2 | CASP1 | 0.783 |
| TNFSF10 | PPP1R3D | 0.783 |
| IL2RG | TSTD1 | 0.783 |
| HLA-B | SERTAD1 | 0.783 |
| SAMD9 | GBP3 | 0.783 |
| CCR1 | GBP1 | 0.783 |
| ATF5 | PARP14 | 0.783 |
| TMUB2 | PRDX5 | 0.783 |
| TLNRD1 | SIGLEC10 | 0.782 |
| SAMD9L | PARP9 | 0.782 |
| KLHDC7B | GBP3 | 0.782 |
| SLC16A3 | CCR1 | 0.782 |
| C6orf47 | IL2RG | 0.782 |
| CD7 | DUSP6 | 0.782 |
| TOR1B | TRIM22 | 0.782 |
| ATF5 | PRDX5 | 0.782 |
| SLC16A3 | NADK | 0.782 |
| CD7 | TMUB2 | 0.782 |
| SLC16A3 | ALDH3A1 | 0.782 |
| C6orf47 | ALDH3A1 | 0.782 |
| ATF5 | PNMA1 | 0.782 |
| SERTAD1 | NADK | 0.782 |
| HLA-A | GBP3 | 0.782 |
| BST2 | APOBEC3G | 0.782 |
| IL2RG | ATF5 | 0.782 |
| CTSL | RIPK3 | 0.782 |
| C6orf47 | TSTD1 | 0.782 |
| NADK | MX2 | 0.782 |
| TRIM22 | EIF2AK2 | 0.782 |
| SOCS1 | RTP4 | 0.782 |
| CDKN1C | TMEM199 | 0.781 |
| NADK | CCR1 | 0.781 |
| HELZ2 | GBP1 | 0.781 |
| CD7 | TPRG1L | 0.781 |
| APOBEC3G | TSTD1 | 0.781 |
| TMUB2 | PNMA1 | 0.781 |
| TMSB10 | ATF5 | 0.781 |
| TYMP | SIGLEC10 | 0.781 |
| SIGLEC10 | EIF2AK2 | 0.781 |
| DUSP6 | ALDH3A1 | 0.781 |
| H4C8 | SIGLEC10 | 0.781 |
| HLA-B | TRIM22 | 0.781 |
| CTSL | GBP3 | 0.781 |
| SOCS1 | TNFSF10 | 0.781 |
| DUSP6 | PPP1R3D | 0.781 |
| ISG20 | GBP1 | 0.781 |
| SOCS1 | PARP14 | 0.781 |
| RIPK3 | CCDC190 | 0.781 |
| SOCS1 | PARP9 | 0.781 |
| H4C8 | SHFL | 0.781 |
| PPP1R3D | KLHDC7B | 0.781 |
| IL2RG | PRDX5 | 0.781 |
| SERTAD1 | INPP5E | 0.781 |
| H4C8 | GBP1 | 0.780 |
| TAP1 | HLA-E | 0.780 |
| SIGLEC10 | ISG20 | 0.780 |
| SERTAD1 | ALDH3A1 | 0.780 |
| HLA-B | TMEM199 | 0.780 |
| HLA-B | MX2 | 0.780 |
| CD7 | PRDX5 | 0.780 |
| SAMD9L | APOBEC3G | 0.780 |

### B. Example 2. mRNA Signature Validation in GSE163151

### 1. Data Set

The 88 mRNA signature of Example 1 was validated in GSE163151. This dataset contains 351 nasopharyngeal (NP) swab samples, taken from patients with COVID-19 (caused by severe acute respiratory syndrome coronavirus 2, SARS-COV-2), patients with various other infections, and healthy donors. [Ng et al. A diagnostic host response biosignature for COVID-19 from RNA profiling of nasal swabs and blood, Science Advances, 7(6) eabe5984 (2021)]. The samples were transcriptomically profiled using RNA-Seq.

### 2. Methods

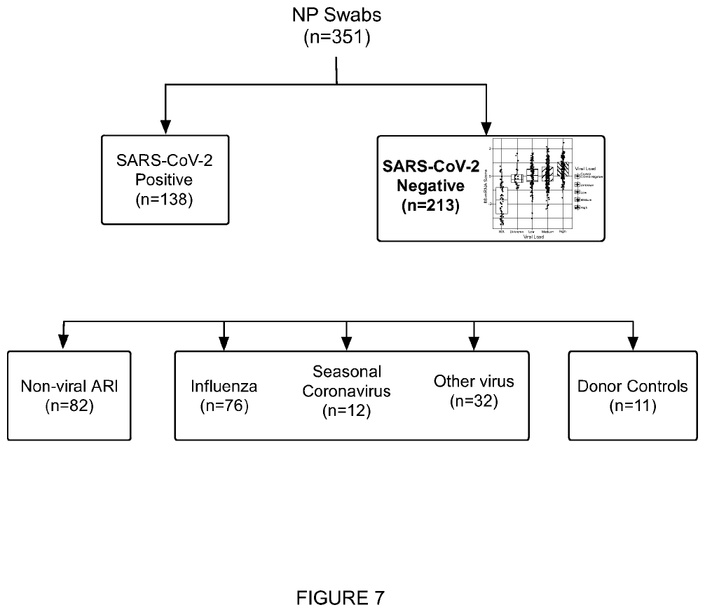

The genome-wide dataset, GSE163151, was downloaded from GEO. We performed a voom transform and further processing. For the 351 swab samples, we further labeled each sample according to its accompanying phenotypic data in GEO. Specifically, SARS-COV-2 positive (n=138), influenza-infected (n=76), seasonal coronavirus (n=12), and other virus-infected (n=32) were assigned as the positive group. Non-viral acute respiratory illness (ARI) (n=82) and healthy control donors (n=11) were assigned as the negative group, as shown in FIG. 7.

### 3. Results

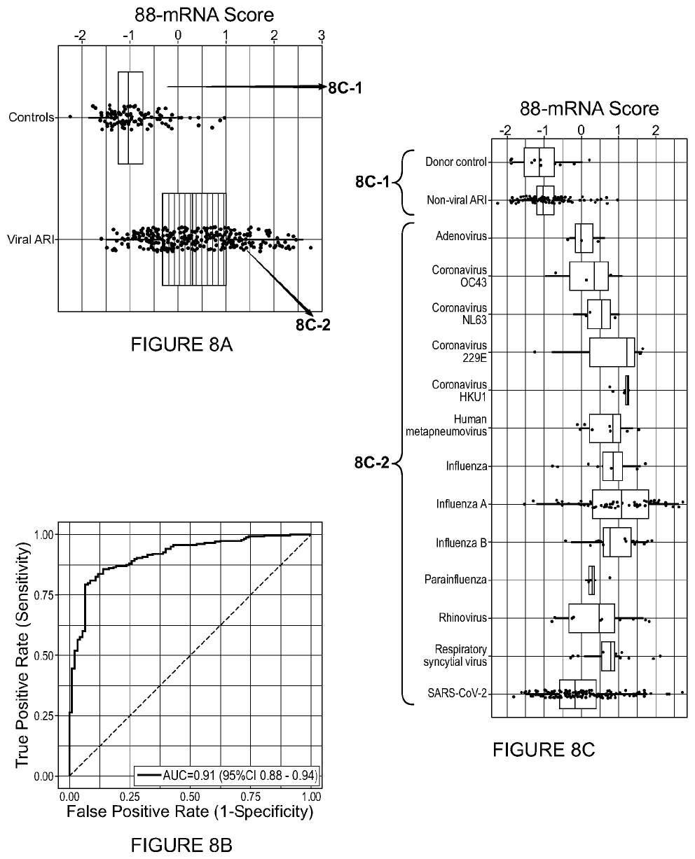

For each sample, we selected the subset of processed RNA-seq data matching our 88-mRNA signature. We then calculated the geometric-mean-based score for each sample. The results are shown in FIG. 8 as boxplot plots (FIG. 8A) and ROC curve (FIG. 8B). It is evident that the score shows a significant separation between the positive group (n=258 total) and the negative group (n=93 total). The AUC for the ROC curve in FIG. 8B is 0.91 (with 95% CL 0.88-0.94). GSE163151 was not used in Example 1 and serves as an independent validation from clinical studies for viral infections, including viruses routinely seen in the clinics and COVID-19.

We further divided the samples by their virus type into 15 sub-groups as shown in FIG. 8C and showed that our 88-mRNA-based score works for various types of viruses captured in this study including SARS-COV-2. Noticeably, the negative group, not only healthy donors but also those with the non-viral ARI showed the clear separation from those virus-infected patients. In clinical practice, distinguishing those with viral infection from non-viral ARI patients may be more clinically meaningful and technically more challenging than from the healthy controls. In this dataset, our 88-mRNA-based score achieved this, demonstrating its utility as a diagnostic test in clinical settings.

### C. Example 3. Additional mRNA Signature Validation in GSE152075

### 1. Data Sets

The 88 mRNA signature of Example 1 was further validated in GSE152075. This dataset contains nasopharyngeal (NP) swab samples taken from 430 patients with COVID-19 of various viral loads and 54 healthy donors without infection [Lieberman et al. In vivo antiviral host transcriptional response to SARS-COV-2 by viral load, sex, and age, PLOS Biology, 18(9) e3000849 (2020)]. The samples were transcriptomically profiled using RNA-Seq.

### 2. Methods

The genome-wide dataset, GSE152075, was downloaded from GEO. We performed a voom transform and further processing. Of 430 COVID-19 patients, the study further divided them based on viral loads in 4 groups: low (n=99), medium (n=206), high (n=108), and unknown (n=17).

### 3. Results

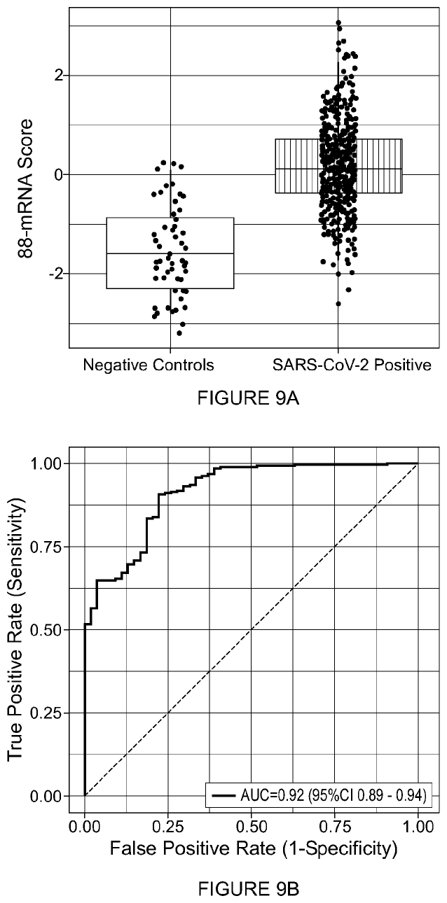

For each sample, we selected the subset of processed RNA-seq data matching our 88 genes. We then calculated the geometric-mean-based score for each sample. The results are shown in FIG. 9 as boxplot plots (FIG. 9A) and ROC curve (FIG. 9B). It is evident that the score shows a significant separation between the positive group (430 COVID-19 patients) and the negative group (54 healthy donors). The AUC for the ROC curve in FIG. 9B is 0.92 (with 95% CL 0.89-0.94). GSE152075 was not used in Example 1 for the discovery of the signature and these results serve as additional independent validation of the 88 mRNA signature, specifically for COVID-19 infected patients.

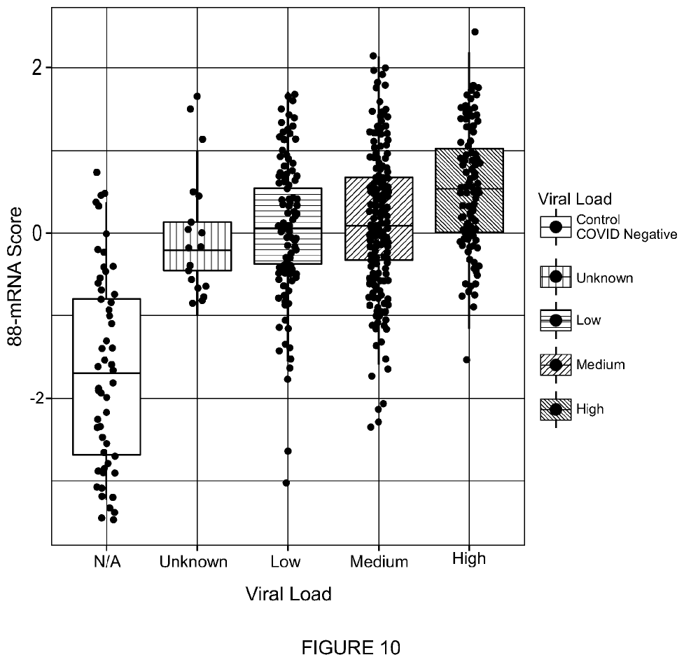

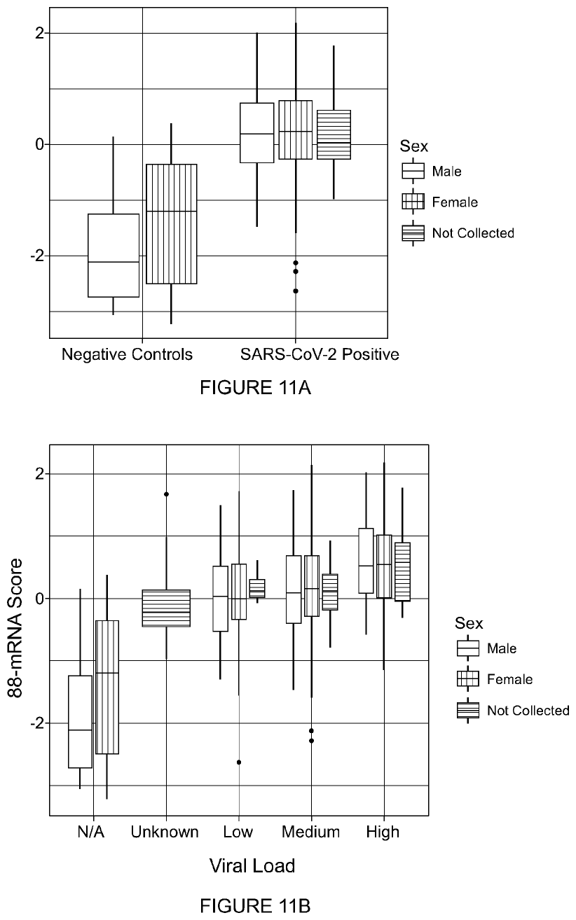

We further divided the samples by their viral load groups as reported in the study and examined the dependence of our 88-mRNA based score on the viral load as shown in FIG. 10. The score illustrates a slight, monotonic dependence on the viral loads; but most importantly was the separability of infected groups even with small viral loads from the uninfected controls. Finally, we found an unbiased performance of our scores in FIG. 11 when samples were divided based only sex for all viral loads taken together (FIG. 11A) and for the various viral load groups (FIG. 11B), further demonstrating the utility of our mRNA signature for the diagnostic use in clinical settings.

The above description of example embodiments of the present disclosure has been presented for the purposes of illustration and description. It is not intended to be exhaustive or to limit the disclosure to the precise form described, and many modifications and variations are possible in light of the teaching above.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

1. A method of administering medical care to a subject presenting one or more symptoms of a respiratory viral infection, the method comprising:
(i) obtaining a respiratory sample from the subject;
(ii) measuring expression levels of one or more biomarkers in the sample, wherein the one or more biomarkers comprise at least one biomarker from Table 2 or Table 3, or one pair of biomarkers from Table 4; and
(iii) generating a viral score based on the measured expression levels of the biomarkers in the sample, wherein a viral score that exceeds a threshold value indicates that the subject has a viral infection.

2. The method of claim 1, wherein the one or more biomarkers comprise at least one biomarker from Table 3.

3. The method of claim 1, wherein the one or more biomarkers comprise at least one pair of biomarkers from Table 4.

4. The method of claim 1, further comprising:
(iv) determining that the subject has a viral infection based on the viral score exceeding the threshold value; and
(v) administering medical care to the subject to treat the viral infection based on the viral score.

5. The method of claim 1, further comprising:
(iv) determining that the subject does not have a viral infection based on the viral score not exceeding the threshold.

6. The method of claim 1, wherein the respiratory sample is selected from the group consisting of nasal, nasopharyngeal, oropharyngeal, oral, or saliva sample.

7. The method of claim 1, further comprising detecting the presence or absence of one or more viruses in the sample.

8. The method of claim 7, wherein the presence or absence of the one or more viruses in the sample is detected using a nucleic acid amplification test (NAAT).

9. The method of claim 1, wherein the expression of the biomarkers is detected using qRT-PCR or isothermal amplification, and wherein the isothermal amplification method is qRT-LAMP.

10. (canceled)

11. (canceled)

12. The method of claim 1, wherein the method comprises measuring the expression of a set of biomarkers in the sample, the set of biomarkers comprising IFITM1, TLNRD1, CDKN1C, INPP5E, and TSTD1.

13. (canceled)

14. The method of claim 4, wherein the medical care comprises administering organ-supportive therapy, administering a therapeutic drug, admitting the subject to an ICU or other hospital ward, or administering a blood product.

15. The method of claim 14, wherein the organ-supportive therapy comprises connecting the subject to any one or more of a mechanical ventilator, a pacemaker, a defibrillator, a dialysis or a renal replacement therapy machine, or an invasive monitor selected from the group consisting of a pulmonary artery catheter, arterial blood pressure catheter, and central venous pressure catheter.

16. The method of claim 14, wherein the therapeutic drug comprises an immune modulator, an antiviral agent, a coagulation modulator, a vasopressor, or a sedative.

17. The method of claim 1, wherein the respiratory viral infection is selected from the group consisting of adenovirus, coronavirus, human metapneumovirus, human rhinovirus (HRV), influenza, parainfluenza, picornavirus, and respiratory syncytial virus (RSV).

18. (canceled)

19. A test kit for detecting the expression levels of one or more biomarkers in a respiratory sample from a subject with one or more symptoms of a respiratory viral infection, wherein the biomarkers comprise at least one biomarker from Table 2 or Table 3, or one pair of biomarkers from Table 4.

20. (canceled)

21. (canceled)

22. The test kit of claim 19, wherein the biomarkers comprise IFITM1, TLNRD1, CDKN1C, INPP5E, and TSTD1.

23. The test kit of claim 22, wherein the kit comprises an oligonucleotide that hybridizes to IFITM1, an oligonucleotide that hybridizes to TLNRD1, an oligonucleotide that hybridizes to CDKN1C, an oligonucleotide that hybridizes to INPP5E, and an oligonucleotide that hybridizes to TSTD1.

24. The test kit of claim 19, wherein the kit is for detecting 1, 2, 3, 4, 5, 6, 7, 8, 9, 10, or more biomarkers.

25. The test kit of claim 19, further comprising one or more reagents for performing q-RT-PCR, qRT-LAMP, or NanoString nCounter analysis.

26. (canceled)

27. The test kit of claim 19, further comprising instructions to calculate a viral score based on the levels of expression of the biomarkers in the respiratory sample from the subject, the score correlating with the likelihood that the subject has a respiratory viral infection.

28. (canceled)

29. (canceled)

30. (canceled)

31. (canceled)

32. (canceled)
